SEQUENCE LISTING (60 sequences)

SEQ ID NO: 1 - DNA, 2341 bp, Homo sapiens
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcata

SEQ ID NO: 2 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 3 - DNA, 2398 bp, Homo sapiens
gggaggccggatgtgagtggagcggccatttcctgtttctctgcagttttcctcagctttgggtggtggccgctgccgggcatcggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagttggagaaacgtacggtaaggatataacctcccggggcaaagacaagccgattgccgatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaaaaaaaaacaaaaaaaaaaaacaaaaaaaaaaaacaacggtggagccttcgcactcaatgccaactttttgttacagattaatttttccataaaaccattttttgaaccaatcagtaattttaaggttttgtttgttctaaatgtaagagttcagactcacattctattaaaatttagccctaaaatgacaagccttcttaaagccttatttttcaaaagcgccccccccattcttgttcagattaagagttgccaaaataccttctgaactacactgcattgttgtgccgagaacaccgagcactgaactttgcaaagaccttcgtctttgagaagacggtagcttctgcagttaggaggtgcagacacttgctctcctatgtagttctcagatgcgtaaagcagaacagcctcccgaatgaagcgttgccattgaactcaccagtgagttagcagcacgtgttcccgacataacattgtactgtaatggagtgagcgtagcagctcagctctttggatcagtctttgtgatttcatagcgagttttctgaccagcttttgcggagattttgaacagaactgctatttcctctaatgaagaattctgtttagctgtgggtgtgccgggtggggtgtgtgtgatcaaaggacaaagacagtattttgacaaaatacgaagtggagatttacactacattgtacaaggaatgaaagtgtcacgggtaaaaactctaaaaggttaatttctgtcaaatgcagtagatgatgaaagaaaggttggtattatcaggaaatgttttcttaagcttttcctttctcttacacctgccatgcctccccaaattgggcatttaattcatctttaaactggttgttctgttagtcgctaacttagtaagtgcttttcttatagaaccccttctgactgagcaatatgcctccttgtattataaaatctttctgataatgcattagaaggtttttttgtcgattagtaaaagtgctttccatgttactttattcagagctaataagtgctttccttagttttctagtaactaggtgtaaaaatcatgtgttgcagctttatagtttttaaaatattttagataattcttaaactatgaaccttcttaacatcactgtcttgccagattaccgacactgtcacttgaccaatactgaccctctttacctcgcccacgcggacacacgcctcctgtagtcgctttgcctattgatgttcctttgggtctgtgaggttctgtaaactgtgctagtgctgacgatgttctgtacaacttaactcactggcgagaatacagcgtgggacccttcagccactacaacagaattttttaaattgacagttgcagaattgtggagtgtttttacattgatcttttgctaatgcaattagcattatgttttgcatgtatgacttaataaatccttgaatcata

SEQ ID NO: 4 - protein, 211 aa, Homo sapiens
MQAIKCVVVGDGAVGKTCLLISYTTNAFPGEYIPTVFDNYSANVMVDGKPVNLGLWDTAGQEDYDRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 5 - DNA, 1516 bp, Homo sapiens
tgccccaccaccgctgctcctcagcaggcgcctcaccagcctccacaccccttgcgcccgcagaaacgcgcctggccctgagctgtcaccaccgacactctccaggctccggacacgatgcaggccatcaagtgtgtggtggtgggagatggggccgtgggcaagacctgccttctcatcagctacaccaccaacgcctttcccggagagtacatccccaccgtgtttgacaactattcagccaatgtgatggtggacagcaagccagtgaacctggggctgtgggacactgctgggcaggaggactacgaccgtctccggccgctctcctatccacagacggacgtcttcctcatctgcttctccctcgtcagcccagcctcttatgagaacgtccgcgccaagtggttcccagaagtgcggcaccactgccccagcacacccatcatcctggtgggcaccaagctggacctgcgggacgacaaggacaccatcgagaaactgaaggagaagaagctggctcccatcacctacccgcagggcctggcactggccaaggagattgactcggtgaaatacctggagtgctcagctctcacccagagaggcctgaaaaccgtgttcgacgaggccatccgggccgtgctgtgccctcagcccacgcggcagcagaagcgcgcctgcagcctcctctaggggttgcaccccagcgctcccacctagatgggtctgatcctccaggatccccacccaaagcctgatggcaccccggctggccatgctgtcccctccctgtggcgtttcttagcagatggctgcagagcttcgttgatggtcttttctgtactggaggcctcctgaggccaggaacgtgcaaatttgcaggtgctgcatcccaagcccctcatgctcctgccttcctgagggccagaggggagccccaggacccattaagccacccccgtgttcctgccgtcagtgccaactgccgcatgtggaagcatctacccgttcactccagtcccaccccacgcctgactcccctctggaaactgcaggccagatggttgctgccacaacttgtgtaccttcagggatggggctcttactccctcctgaggccagctgctctaatatcgatggtcctgcttgccagagagttcctctacccagcaaaaatgagtgtctcagaagtgtgctcctctggcctcagttctcctcttttggaacaacataaaacaaatttaattttctacgcctctggggatatctgctcagccaatggaaaatctgggttcaaccagcccctgccatttcttaagactttctgctgcactcacaggatcctgagctgcacttacctgtgagagtcttcaaacttttaaaccttgccagtcaggacttttgctattgcaaatagaaaacccaactcaacctgcttaagcagaaaataaatttattgattcaaaaaaaaaaaaaaaaaaaaaaaaaaaaaaaaaaaaaaaaaaaaaaaaaa

SEQ ID NO: 6 - protein, 192 aa, Homo sapiens
MQAIKCVVVGDGAVGKTCLLISYTTNAFPGEYIPTVFDNYSANVMVDSKPVNLGLWDTAGQEDYDRLRPLSYPQTDVFLICFSLVSPASYENVRAKWFPEVRHHCPSTPIILVGTKLDLRDDKDTIEKLKEKKLAPITYPQGLALAKEIDSVKYLECSALTQRGLKTVFDEAIRAVLCPQPTRQQKRACSLL

SEQ ID NO: 7 - DNA, 1077 bp, Homo sapiens
tgtctccggccgatcgctcggcgctcgggtccgcggccgctgcggcgccgggcatttctccgcagctcggctcgcggccgcgcccgccgccgcccggcccgcgcccatgcaggccatcaagtgcgtggtggtcggcgacggcgccgtggggaagacatgcttgctgatcagctacacgaccaacgccttccccggagagtacatccccaccgtttttgacaactactctgccaacgtgatggtggacgggaaaccagtcaacttggggctgtgggacacagcgggtcaggaggactacgatcggctgcggccactctcctacccccaaactgacgtctttctgatctgcttctctctggtgagcccggcctccttcgagaatgttcgtgccaagtggtacccggaggtgcggcaccactgcccccacacgcccatcctcctggtgggcaccaagctggacctccgcgacgacaaggacaccattgagcggctgcgggacaagaagctggcacccatcacctacccacagggcctggccatggcccgggagattggctctgtgaaatacctggagtgctcagccctgacccagcggggcctgaagacagtgtttgacgaggcgatccgcgcggtgctctgcccgcccccagtgaagaagccggggaagaagtgcaccgtcttctagagccctggcccacccgagcctgagggctggcggggagcagccctggacgtgtccgctgttgtgttgagacgtgtggtgtccctgagtcggctgtggggagcggtgggggtgggccggggggaagcatggggatgaggctgggtggcaggatcctgtcctctctgccgcctcattctggggtgtggctccagccttccctggcccccgccggaggccgggagggagcagggtctccctcagggctgcaggggcaggtgcagggaagccccaggatgggcttccctggagggggagggtgggggggagttctgttccttgtgccccgaggtggggcagccccttctcattttatacaataaacattctccacctacaaaaaaaaaaaaaaaaaa

SEQ ID NO: 8 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPHTPILLVGTKLDLRDDKDTIERLRDKKLAPITYPQGLAMAREIGSVKYLECSALTQRGLKTVFDEAIRAVLCPPPVKKPGKKCTVF

SEQ ID NO: 9 - DNA, 1662 bp, Homo sapiens
atgtcaaataacggcctagacattcaagacaaacccccagcccctccgatgagaaataccagcactatgattggagccggcagcaaagatgctggaaccctaaaccatggttctaaacctctgcctccaaacccagaggagaagaaaaagaaggaccgattttaccgatccattttacctggagataaaacaaataaaaagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagctgaggattacaattcttctaatgccttgaatgtgaaggctgtgtctgagactcctgcagtgccaccagtttcagaagatgaggatgatgatgatgatgatgctaccccaccaccagtgattgctccacgcccagagcacacaaaatctgtatacacacggtctgtgattgaaccacttcctgtcactccaactcgggacgtggctacatctcccatttcacctactgaaaataacaccactccaccagatgctttgacccggaatactgagaagcagaagaagaagcctaaaatgtctgatgaggagatcttggagaaattacgaagcatagtgagtgtgggcgatcctaagaagaaatatacacggtttgagaagattggacaaggtgcttcaggcaccgtgtacacagcaatggatgtggccacaggacaggaggtggccattaagcagatgaatcttcagcagcagcccaagaaagagctgattattaatgagatcctggtcatgagggaaaacaagaacccaaacattgtgaattacttggacagttacctcgtgggagatgagctgtgggttgttatggaatacttggctggaggctccttgacagatgtggtgacagaaacttgcatggatgaaggccaaattgcagctgtgtgccgtgagtgtctgcaggctctggagttcttgcattcgaaccaggtcattcacagagacatcaagagtgacaatattctgttgggaatggatggctctgtcaagctaactgactttggattctgtgcacagataaccccagagcagagcaaacggagcaccatggtaggaaccccatactggatggcaccagaggttgtgacacgaaaggcctatgggcccaaggttgacatctggtccctgggcatcatggccatcgaaatgattgaaggggagcctccatacctcaatgaaaaccctctgagagccttgtacctcattgccaccaatgggaccccagaacttcagaacccagagaagctgtcagctatcttccgggactttctgaaccgctgtctcgagatggatgtggagaagagaggttcagctaaagagctgctacaggtgagaaaactgaggtttcaagtgtttagtaacttttccatgatagctgcatcaattcctgaagattgccaagcccctctccagcctcactccactgattgctgcagctaa

SEQ ID NO: 10 - protein, 553 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQVRKLRFQVFSNFSMIAASIPEDCQAPLQPHSTDCCS

SEQ ID NO: 11 - DNA, 1638 bp, Homo sapiens
atgtcaaataacggcctagacattcaagacaaacccccagcccctccgatgagaaataccagcactatgattggagccggcagcaaagatgctggaaccctaaaccatggttctaaacctctgcctccaaacccagaggagaagaaaaagaaggaccgattttaccgatccattttacctggagataaaacaaataaaaagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagctgaggattacaattcttctaatgccttgaatgtgaaggctgtgtctgagactcctgcagtgccaccagtttcagaagatgaggatgatgatgatgatgatgctaccccaccaccagtgattgctccacgcccagagcacacaaaatctgtatacacacggtctgtgattgaaccacttcctgtcactccaactcgggacgtggctacatctcccatttcacctactgaaaataacaccactccaccagatgctttgacccggaatactgagaagcagaagaagaagcctaaaatgtctgatgaggagatcttggagaaattacgaagcatagtgagtgtgggcgatcctaagaagaaatatacacggtttgagaagattggacaaggtgcttcaggcaccgtgtacacagcaatggatgtggccacaggacaggaggtggccattaagcagatgaatcttcagcagcagcccaagaaagagctgattattaatgagatcctggtcatgagggaaaacaagaacccaaacattgtgaattacttggacagttacctcgtgggagatgagctgtgggttgttatggaatacttggctggaggctccttgacagatgtggtgacagaaacttgcatggatgaaggccaaattgcagctgtgtgccgtgagtgtctgcaggctctggagttcttgcattcgaaccaggtcattcacagagacatcaagagtgacaatattctgttgggaatggatggctctgtcaagctaactgactttggattctgtgcacagataaccccagagcagagcaaacggagcaccatggtaggaaccccatactggatggcaccagaggttgtgacacgaaaggcctatgggcccaaggttgacatctggtccctgggcatcatggccatcgaaatgattgaaggggagcctccatacctcaatgaaaaccctctgagagccttgtacctcattgccaccaatgggaccccagaacttcagaacccagagaagctgtcagctatcttccgggactttctgaaccgctgtctcgagatggatgtggagaagagaggttcagctaaagagctgctacagcatcaattcctgaagattgccaagcccctctccagcctcactccactgattgctgcagctaaggaggcaacaaagaacaatcactaa

SEQ ID NO: 12 - protein, 545 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH

SEQ ID NO: 13 - DNA, 4065 bp, Homo sapiens
atgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaa

SEQ ID NO: 14 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 15 - DNA, 4167 bp, Homo sapiens
atgagccggcccccgccgacggggaaaatgcccggcgcccccgagaccgcgccgggggacggggcaggcgcgagccgccagaggaagctggaggcgctgatccgagaccctcgctcccccatcaacgtggagagcttgctggatggcttaaattccttggtccttgatttagattttcctgctttgaggaaaaacaagaacatagataatttcttaaatagatatgagaaaattgtgaaaaaaatcagaggtctacagatgaaggcagaagactatgatgttgtaaaagttattggaagaggtgcttttggtgaagtgcagttggttcgtcacaaggcatcgcagaaggtttatgctatgaagcttcttagtaagtttgaaatgataaaaagatcagattctgcctttttttgggaagaaagagatattatggcctttgccaatagcccctgggtggttcagcttttttatgcctttcaagatgataggtatctgtacatggtaatggagtacatgcctggtggagaccttgtaaaccttatgagtaattatgatgtgcctgaaaaatgggccaaattttacactgctgaagttgttcttgctctggatgcaatacactccatgggtttaatacacagagatgtgaagcctgacaacatgctcttggataaacatggacatctaaaattagcagattttggcacgtgtatgaagatggatgaaacaggcatggtacattgtgatacagcagttggaacaccggattatatatcacctgaggttctgaaatcacaagggggtgatggtttctatgggcgagaatgtgattggtggtctgtaggtgttttcctttatgagatgctagtgggggatactccattttatgcggattcacttgtaggaacatatagcaaaattatggatcataagaattcactgtgtttccctgaagatgcagaaatttccaaacatgcaaagaatctcatctgtgctttcttaacagatagggaggtacgacttgggagaaatggggtggaagaaatcagacagcatcctttctttaagaatgatcagtggcattgggataacataagagaaacggcagctcctgtagtacctgaactcagcagtgacatagacagcagcaatttcgatgacattgaagatgacaaaggagatgtagaaaccttcccaattcctaaagcttttgttggaaatcagctgcctttcatcggatttacctactatagagaaaatttattattaagtgactctccatcttgtagagaaactgattccatacaatcaaggaaaaatgaagaaagtcaagagattcagaaaaaactgtatacattagaagaacatcttagcaatgagatgcaagccaaagaggaactggaacagaagtgcaaatctgttaatactcgcctagaaaaaacagcaaaggagctagaagaggagattaccttacggaaaagtgtggaatcagcattaagacagttagaaagagaaaaggcgcttcttcagcacaaaaatgcagaatatcagaggaaagctgatcatgaagcagacaaaaaacgaaatttggaaaatgatgttaacagcttaaaagatcaacttgaagatttgaaaaaaagaaatcaaaactctcaaatatccactgagaaagtgaatcaactccagagacaactggatgaaaccaatgctttactgcgaacagagtctgatactgcagcccggttaaggaaaacccaggcagaaagttcaaaacagattcagcagctggaatctaacaatagagatctacaagataaaaactgcctgctggagactgccaagttaaaacttgaaaaggaatttatcaatcttcagtcagctctagaatctgaaaggagggatcgaacccatggatcagagataattaatgatttacaaggtagaatatgtggcctagaagaagatttaaagaacggcaaaatcttactagcgaaagtagaactggagaagagacaacttcaggagagatttactgatttggaaaaggaaaaaagcaacatggaaatagatatgacataccaactaaaagttatacagcagagcctagaacaagaagaagctgaacataaggccacaaaggcacgactagcagataaaaataagatctatgagtccatcgaagaagccaaatcagaagccatgaaagaaatggagaagaagctcttggaggaaagaactttaaaacagaaagtggagaacctattgctagaagctgagaaaagatgttctctattagactgtgacctcaaacagtcacagcagaaaataaatgagctccttaaacagaaagatgtgctaaatgaggatgttagaaacctgacattaaaaatagagcaagaaactcagaagcgctgccttacacaaaatgacctgaagatgcaaacacaacaggttaacacactaaaaatgtcagaaaagcagttaaagcaagaaaataaccatctcatggaaatgaaaatgaacttggaaaaacaaaatgctgaacttcgaaaagaacgtcaggatgcagatgggcaaatgaaagagctccaggatcagctcgaagcagaacagtatttctcaaccctttataaaacacaagttagggagcttaaagaagaatgtgaagaaaagaccaaacttggtaaagaattgcagcagaagaaacaggaattacaggatgaacgggactctttggctgcccaactggagatcaccttgaccaaagcagattctgagcaactggctcgttcaattgctgaagaacaatattctgatttggaaaaagagaagatcatgaaagagctggagatcaaagagatgatggctagacacaaacaggaacttacggaaaaagatgctacaattgcttctcttgaggaaactaataggacactaactagtgatgttgccaatcttgcaaatgagaaagaagaattaaataacaaattgaaagatgttcaagagcaactgtcaagattgaaagatgaagaaataagcgcagcagctattaaagcacagtttgagaagcagctattaacagaaagaacactcaaaactcaagctgtgaataagttggctgagatcatgaatcgaaaagaacctgtcaagcgtggtaatgacacagatgtgcggagaaaagagaaggagaatagaaagctacatatggagcttaaatctgaacgtgagaaattgacccagcagatgatcaagtatcagaaagaactgaatgaaatgcaggcacaaatagctgaagagagccagattcgaattgaactgcagatgacattggacagtaaagacagtgacattgagcagctgcggtcacaactccaagccttgcatattggtctggatagttccagtataggcagtggaccaggggatgctgaggcagatgatgggtttccagaatcaagattagaaggatggctttcattgcctgtacgaaacaacactaagaaatttggatgggttaaaaagtatgtgattgtaagcagtaagaagattcttttctatgacagtgaacaagataaagaacaatccaatccttacatggttttagatatagacaagttatttcatgtccgaccagttacacagacagatgtgtatagagcagatgctaaagaaattccaaggatattccagattctgtatgccaatgaaggagaaagtaagaaggaacaagaatttccagtggagccagttggagaaaaatctaattatatttgccacaagggacatgagtttattcctactctttatcatttcccaaccaactgtgaggcttgtatgaagcccctgtggcacatgtttaagcctcctcctgctttggagtgccgccgttgccatattaagtgtcataaagatcatatggacaaaaaggaggagattatagcaccttgcaaagtatattatgatatttcaacggcaaagaatctgttattactagcaaattctacagaagagcagcagaagtgggttagtcggttggtgaaaaagatacctaaaaagcccccagctccagacccttttgcccgatcatctcctagaacttcaatgaagatacagcaaaaccagtctattagacggccaagtcgacagcttgccccaaacaaacctagctaa

SEQ ID NO: 16 - protein, 1388 aa, Homo sapiens
MSRPPPTGKMPGAPETAPGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYRENLLLSDSPSCRETDSIQSRKNEESQEIQKKLYTLEEHLSNEMQAKEELEQKCKSVNTRLEKTAKELEEEITLRKSVESALRQLEREKALLQHKNAEYQRKADHEADKKRNLENDVNSLKDQLEDLKKRNQNSQISTEKVNQLQRQLDETNALLRTESDTAARLRKTQAESSKQIQQLESNNRDLQDKNCLLETAKLKLEKEFINLQSALESERRDRTHGSEIINDLQGRICGLEEDLKNGKILLAKVELEKRQLQERFTDLEKEKSNMEIDMTYQLKVIQQSLEQEEAEHKATKARLADKNKIYESIEEAKSEAMKEMEKKLLEERTLKQKVENLLLEAEKRCSLLDCDLKQSQQKINELLKQKDVLNEDVRNLTLKIEQETQKRCLTQNDLKMQTQQVNTLKMSEKQLKQENNHLMEMKMNLEKQNAELRKERQDADGQMKELQDQLEAEQYFSTLYKTQVRELKEECEEKTKLGKELQQKKQELQDERDSLAAQLEITLTKADSEQLARSIAEEQYSDLEKEKIMKELEIKEMMARHKQELTEKDATIASLEETNRTLTSDVANLANEKEELNNKLKDVQEQLSRLKDEEISAAAIKAQFEKQLLTERTLKTQAVNKLAEIMNRKEPVKRGNDTDVRRKEKENRKLHMELKSEREKLTQQMIKYQKELNEMQAQIAEESQIRIELQMTLDSKDSDIEQLRSQLQALHIGLDSSSIGSGPGDAEADDGFPESRLEGWLSLPVRNNTKKFGWVKKYVIVSSKKILFYDSEQDKEQSNPYMVLDIDKLFHVRPVTQTDVYRADAKEIPRIFQILYANEGESKKEQEFPVEPVGEKSNYICHKGHEFIPTLYHFPTNCEACMKPLWHMFKPPPALECRRCHIKCHKDHMDKKEEIIAPCKVYYDISTAKNLLLLANSTEEQQKWVSRLVKKIPKKPPAPDPFARSSPRTSMKIQQNQSIRRPSRQLAPNKPS

SEQ ID NO: 17 - DNA, 24 bp, Artificial
  Synthesized primer.
agttttcctcagctttgggtggtg

SEQ ID NO: 18 - DNA, 24 bp, Artificial
  Synthesized primer.
aaagcgtacaaaggttccaaggga

SEQ ID NO: 19 - DNA, 25 bp, Artificial
  Synthesized primer.
tcagggtaccaatgtgtatgtggtg

SEQ ID NO: 20 - DNA, 24 bp, Artificial
  Synthesized primer.
tggtcaaagaaatgtgaaacccgt

SEQ ID NO: 21 - DNA, 24 bp, Artificial
  Synthesized primer.
ccttcccagcaacatgtagaaagc

SEQ ID NO: 22 - DNA, 24 bp, Artificial
  Synthesized primer.
cagcctggacacaacagagtgaga

SEQ ID NO: 23 - DNA, 24 bp, Artificial
  Synthesized primer.
ctgagctgtcaccaccgacactct

SEQ ID NO: 24 - DNA, 24 bp, Artificial
  Synthesized primer.
agctctgcagccatctgctaagaa

SEQ ID NO: 25 - DNA, 24 bp, Artificial
  Synthesized primer.
cttctaccccttcctccatacccc

SEQ ID NO: 26 - DNA, 24 bp, Artificial
  Synthesized primer.
ccctcttgcacttcctgtctttca

SEQ ID NO: 27 - DNA, 20 bp, Artificial
  Synthesized primer.
atttctccgcagctcggctc

SEQ ID NO: 28 - DNA, 24 bp, Artificial
  Synthesized primer.
ggacaccacacgtctcaacacaac

SEQ ID NO: 29 - DNA, 44 bp, Artificial
  Synthesized primer.
ctgatcagttacacaaccaatgcattttctggagaatatatccc

SEQ ID NO: 30 - DNA, 44 bp, Artificial
  Synthesized primer.
gggatatattctccagaaaatgcattggttgtgtaactgatcag

SEQ ID NO: 31 - DNA, 33 bp, Artificial
  Synthesized primer.
gtaaaatacctggagtactcggcgctcacacag

SEQ ID NO: 32 - DNA, 33 bp, Artificial
  Synthesized primer.
ctgtgtgagcgccgagtactccaggtattttac

SEQ ID NO: 33 - DNA, 27 bp, Artificial
  Synthesized primer.
gcagtcctctgcctgcctcccgtgaag

SEQ ID NO: 34 - DNA, 27 bp, Artificial
  Synthesized primer.
cttcacgggaggcaggcagaggactgc

SEQ ID NO: 35 - DNA, 35 bp, Artificial
  Synthesized primer.
gcaagacctgccttctcatgagctacaccaccaac

SEQ ID NO: 36 - DNA, 35 bp, Artificial
  Synthesized primer.
gttggtggtgtagctcatgagaaggcaggtcttgc

SEQ ID NO: 37 - DNA, 36 bp, Artificial
  Synthesized primer.
caccaccaacgcctttcaaggagagtacatccccac

SEQ ID NO: 38 - DNA, 36 bp, Artificial
  Synthesized primer.
gtggggatgtactctccttgaaaggcgttggtggtg

SEQ ID NO: 39 - DNA, 35 bp, Artificial
  Synthesized primer.
cagccaatgtgatggtgtacagcaagccagtgaac

SEQ ID NO: 40 - DNA, 35 bp, Artificial
  Synthesized primer.
gttcactggcttgctgtacaccatcacattggctg

SEQ ID NO: 41 - DNA, 27 bp, Artificial
  Synthesized primer.
cggcaccactgccacagcacacccatc

SEQ ID NO: 42 - DNA, 27 bp, Artificial
  Synthesized primer.
gatgggtgtgctgtggcagtggtgccg

SEQ ID NO: 43 - DNA, 24 bp, Artificial
  Synthesized primer.
aagctgactcccatcacctatccg

SEQ ID NO: 44 - DNA, 24 bp, Artificial
  Synthesized primer.
cgaggggctgagacatttacaaca

SEQ ID NO: 45 - DNA, 24 bp, Artificial
  Synthesized primer.
aagaagctggctcccatcacctac

SEQ ID NO: 46 - DNA, 23 bp, Artificial
  Synthesized primer.
aacacggttttcaggcctctctg

SEQ ID NO: 47 - DNA, 24 bp, Artificial
  Synthesized primer.
aagaagctggcacccatcacctac

SEQ ID NO: 48 - DNA, 24 bp, Artificial
  Synthesized primer.
atcgcctcgtcaaacactgtcttc

SEQ ID NO: 49 - DNA, 24 bp, Artificial
  Synthesized primer.
tcaacagcagtgatgatgggactc

SEQ ID NO: 50 - DNA, 24 bp, Artificial
  Synthesized primer.
agggtgtcagtgcagcttgaaagt

SEQ ID NO: 51 - DNA, 24 bp, Artificial
  Synthesized primer.
tggggagggctttctttgtgtatt

SEQ ID NO: 52 - DNA, 25 bp, Artificial
  Synthesized primer.
tgctaagtcctgagcctgttttgtg

SEQ ID NO: 53 - DNA, 24 bp, Artificial
  Synthesized primer.
agcagatcaaacgggtgaaggact

SEQ ID NO: 54 - DNA, 24 bp, Artificial
  Synthesized primer.
gatctcacgcaccaacgtgtagaa

SEQ ID NO: 55 - RNA, 19 nt, Homo sapiens
uaaggagauuggugcugua

SEQ ID NO: 56 - RNA, 19 nt, Homo sapiens
cggcaccacugucccaaca

SEQ ID NO: 57 - RNA, 19 nt, Homo sapiens
auacgccaguaccgaauga

SEQ ID NO: 58 - RNA, 19 nt, Homo sapiens
aaagcgcacugacaaucca

SEQ ID NO: 59 - DNA, 19 bp, Homo sapiens
taaggagattggtgctgta

SEQ ID NO: 60 - DNA, 19 bp, Artificial
  Synthesized DNA.
caaagaaattggagcagtg

(19) United States
(12) Patent Application Publication
(10) Pub. No.: US 2015/0185223 A1
(43) Pub. Date: Jul. 2, 2015

(54) Compositions Comprising RAC Mutants, and Methods of Use Thereof

(71) Applicants: The University of Tokyo, Tokyo (JP); Japanese Foundation for Cancer Research, Tokyo (JP); Educational Foundation Jichi Medical University, Tochigi (JP)

(72) Inventors: Hiroyuki Mano, Tokyo (JP); Masahito Kawazu, Tokyo (JP); Kengo Takeuchi, Tokyo (JP); Yoshio Miki, Tokyo (JP); Toshihide Ueno, Tokyo (JP)

(21) Appl. No.: 14/417,474

(22) PCT Filed: Jul. 26, 2013
(86) PCT No.: PCT/JP2013/004564

§ 371 Date: Jan. 26, 2015

Related U.S. Application Data
(60) Provisional application No. 61/676,117, filed on Jul. 26, 2012.

(51) Int. Cl. G01N 33/574; C12N 15/113; C12Q 1/68; A61K 31/7088; C12N 9/14; C07K 16/40; A61K 45/06

(52) U.S. Cl. CPC G01N 33/57496; C07K 16/40; C12N 15/1137; A61K 45/06; A61K 31/7088; C12N 9/14; C12Q 1/6886; C12N 2310/14; C12N 2320/31; C12Y 306/05002; C12Q 2600/156; C12Q 2600/158; C12Q 2600/118; G01N 2333/914

(57) ABSTRACT

The present invention is based, in part, on the discovery of isolated nucleic acid molecules encoding mutant RAC polypeptides, or fragments thereof, wherein the mutant RAC polypeptides comprise one or more substitutions of an amino acid in the wild-type RAC polypeptide that renders the mutant RAC polypeptides constitutively active and oncogenic. Isolated mutant RAC polypeptides encoded by such nucleic acid molecules, as well as vectors, host cells, methods of producing encoded polypeptides using such isolated nucleic acid molecules, as well as methods of using mutant RAC nucleic acids and polypeptides for identifying, assessing, prognosing, and treating cancer, are also provided.

### CROSS-REFERENCE TO RELATED APPLICATIONS

This application claims the benefit of U.S. Provisional Application No. 61/676,117, filed Jul. 26, 2012, the entire contents of which are incorporated herein by reference. The invention of the present application is related to Compositions Comprising RAC Mutants, and Methods of Use Thereof.

### TECHNICAL FIELD

### Background Art

The identification of transforming proteins and the development of agents that target them have markedly influenced the treatment and improved the prognosis of individuals with cancer. Chronic myeloid leukemia (CML), for example, has been shown to result from the growth-promoting activity of the fusion tyrosine kinase breakpoint cluster region-Abelson murine leukemia viral oncogene homolog 1 (BCR-ABL1), and treatment with a specific ABL1 inhibitor, imatinib mesylate, has increased the 5-year survival rate of individuals with CML to almost 90% (Druker et al. (2006) N. Engl. J. Med. 355:2408-2417). Similarly, the fusion of echinoderm microtubule associated protein like 4 gene (EML4) to anaplastic lymphoma receptor tyrosine kinase (ALK) is responsible for a subset of non-small-cell lung cancer cases (Soda et al. (2007) Nature 448:561-566), and therapy targeted to EML4-ALK kinase activity has greatly improved the progression-free survival of affected individuals compared with that achieved with conventional chemotherapies (Shaw et al. (2011) Lancet Oncol. 12:1004-1012). In another example, epidermal growth factor receptor (EGFR) becomes constitutively activated through amino acid substitutions or internal deletions among a subset of non-small cell lung cancer, and treatment of such individuals with EGFR inhibitors was shown to significantly prolong their progression-free survival (Mok et al. (2009) N. Engl. J. Med. 361:947-957). Therapies that target essential growth drivers in human cancers are thus among the most effective treatments for these intractable disorders.

V-Ki-ras2 Kirsten rat sarcoma viral oncogene homolog (KRAS), v-Ha-ras Harvey rat sarcoma viral oncogene homolog (HRAS), and neuroblastoma RAS viral (v-ras) oncogene homolog (NRAS) are the founding members of the rat sarcoma (RAS) superfamily of small guanosine triphosphatases (GTPases) that is known to comprise >150 members in humans (Colicelli (2004) Sci STKE 2004:RE13). Five subgroups of these small GTPases have been identified and designated as the RAS; ras homolog family member (RHO); RAB1A, member RAS oncogene family (RAB); RAN, member RAS oncogene family (RAN); and ADP-ribosylation factor (ARF) families. All small GTPases function as binary switches that transition between GDP-bound, inactive and GTP-bound, active forms and thereby contribute to intracellular signaling that underlies a wide array of cellular activities, including cell proliferation, differentiation, survival, motility, cytoskeleton rearrangements, and transformation (Cox and Der (2010) Small GTPases 1:2-27). Somatic point mutations that activate KRAS, HRAS, or NRAS have been identified in a variety of human tumors, with KRAS being the most frequently activated oncoprotein in humans. Somatic activating mutations of KRAS are thus present in >90% of pancreatic adenocarcinomas, for example (Jaffee et al. (2002) Cancer Cell 2:25-28). Surprisingly, however, mutational activation of small GTPases other than KRAS, HRAS, and NRAS has not been widely reported.

Ras-related C3 botulinum toxin substrate (RAC) 1, RAC2, and RAC3 belong to the RHO family of small GTPases (Wertheimer et al. (2012) Cell Signal 24:353-362). RAC proteins orchestrate actin polymerization, and their activation induces the formation of membrane ruffles and lamellipodia (Ridley et al. (1992) Cell 70:401-410), which play essential roles in the maintenance of cell morphology and in cell migration. Accumulating evidence also indicates that RAC proteins function as key hubs of intracellular signaling that underlies cell transformation. RAC1, for example, serves as an essential downstream component of the signaling pathway by which oncogenic RAS induces cell transformation, and artificial introduction of an amino acid substitution (G12V) into RAC1 renders it oncogenic (Qiu et al. (1995) Nature 374:457-459). Furthermore, suppression of RAC1 activity induces apoptosis in glioma cells (Senger et al. (2002) Cancer Res. 62:2131-2140), and loss of RAC1 or RAC2 results in a marked delay in the development of BCR-ABL1-driven myeloproliferative disorder (Thomas et al. (20007) Cancer Cell 12:467-478). Despite such important roles of RAC proteins in cancer, somatic transforming mutations of these proteins have not been identified in cancer specimens. Accordingly, there is a great need in the art to identify such RAC protein mutants.

### SUMMARY OF INVENTION

One aspect of the present invention relates to isolated nucleic acid molecules encoding a mutant RAC polypeptide, or a fragment thereof, wherein the mutant RAC polypeptide comprises one or more substitutions of an amino acid in the wild-type RAC polypeptide that renders the mutant RAC polypeptide constitutively active and oncogenic. For example, the one or more amino acid substitutions can be selected from the group consisting of RAC1(N92I), RAC1(P29S), RAC1(C157Y), RAC1(P179L), RAC2(I121M), RAC2(P29Q), RAC2(D47Y), and RAC2(P106H). Another aspect of the present invention relates to isolated mutant RAC polypeptides encoded by such nucleic acid molecules, as well as vectors, host cells, methods of producing encoded polypeptides using such isolated nucleic acid molecules. Yet another aspect of the present invention relates to methods of using mutant RAC nucleic acids and polypeptides for identifying, assessing, prognosing, and treating cancer.

### BRIEF DESCRIPTION OF DRAWINGS

FIG. 1 shows the sequence of RAC1 and RAC2 mutations. Electrophoretograms of RAC1 cDNAs revealed 516A>T and 326C>T mutations, resulting in N92I and P29S amino acid substitutions, in HT1080 and MDA-MB-157 cells, respectively. Similarly, sequencing of RAC2 cDNAs identified 203_{—}204CC>AA and 203C>T mutations, resulting in P29Q and P29L amino acid changes, in KCL-22 and HCC1143 cells, respectively.

FIGS. 2A-2D show the transforming potential of RAC1 and RAC2 mutants. FIG. 2A shows 3T3 or MCF10A cells infected with recombinant retroviruses encoding enhanced green fluorescent protein (EGFP) as well as wild-type or mutant forms of RAC1 or RAC2 and assayed for anchorage-independent growth in vitro under the presence of 10% (vol/vol) FBS. After 14 d (3T3) or 20 d (MCF10A) of culture, the cells were stained with crystal violet and examined by conventionalmicroscopy (Left: left image of each pair), and they were monitored for EGFP expression by fluorescence microscopy (Left: right image of each pair) (Scalebars, 0.5 mm.) The numbers of cell colonies were also determined as means+/−SD from three independent experiments (Right). FIG. 2B shows 3T3 cells expressing wild-type or mutant forms of RAC1 or RAC2 injected s.c. into the shoulder of nude mice. The size of the resulting tumors [(length×width)/2] was determined at the indicated times thereafter. Tumor size for 3T3 expressing NRAS(Q61K) was similarly monitored. Data are means+/−SD for tumors at fourinjection sites. FIG. 2C shows results of HEK293T cells transfected with expression vectors for wild-type or mutant forms of RAC1 or RAC2 together with the SRE.L reporter plasmid and pGL-TK. The activity of firefly luciferase in cell lysates was then measured and normalized by that of Renilla luciferase. Data are means+/−SD from three independent experiments. FIG. 2D shows the results of lysates of 3T3 cells expressing wild-type or mutant forms of RAC1 or RAC2 subjected to a pull-down assay with PAK1-PBD. The precipitated proteins as well as the total cell lysates were then subjected to immunoblot analysis with antibodies to RAC1 or to RAC2. The relative amounts of pulled-down RAC proteins compared with their corresponding expression levels in total cell lysate were normalized to that of wild-type RAC1 (for the RAC1 mutants) or RAC2 (for the RAC2 mutants) and are shown at the bottom.

FIG. 3 shows the results of introducing GTPases into 3T3 cells. Total cell lysates (10 microgram) obtained from 3T3 cells infected with mock retrovirus or that express NRAS or NRAS (Q61K) were subjected to immunoblot analyses with antibodies to NRAS or ACTB (top panel). Likewise, expression of RAC1 or its mutants (middle panel), or of RAC2 or its mutants (bottom panel) was assessed by immunoblot analyses with corresponding antibodies.

FIG. 4 shows actin reorganization induced by the RAC1/RAC2 mutants. 3T3 cells infected with retroviruses encoding enhanced green fluorescent protein (EGFP) as well as wild-type or mutant forms of RAC1 or RAC2 and were stained with Alexa Fluor 594-labeled phalloidin to visualize actin organization (left image of eachpair). The same cells were also examined for EGFP fluorescence (right image of each pair) (Scale bars, 20 micrometer).

FIGS. 5A-5D show that oncogenic RAC proteins are therapeutic targets. FIG. 5A shows the results of HT1080 cells transfected with control, RAC1, or NRAS siRNAs; lysed; and subjected to immunoblot analysis with antibodies to RAC1, NRAS, or ACTB (loading control). FIG. 5B shows the results of HT1080 cells transfected with control, RAC1, or NRAS siRNAs, as indicated, and cultured under the presence of 10% (vol/vol) FBS. Cell number was counted at the indicated times after the onset of transfection. Data are means+/−SD from three independent experiments. FIG. 5C shows the results of HT1080 cells infected with a retrovirus encoding green fluorescent protein (EGFP) as well as a control or RAC1shRNA. They were also infected with a retrovirus encoding shRNA-resistant wild-type RAC1 or RAC1(N92I), as indicated. The number of EGFP-positive cells wasdetermined by flow cytometry after culture of the cells for the indicated times, and the size of the EGFP-positive fraction relative to that at 2 d was calculated. Data are means+/−SD from three independent experiments for each panel. FIG. 5D shows the results of MDA-MB-157 cells infected with a retrovirus encoding EGFP as well as a control or RAC1 shRNA. They were also infected with a retrovirus encoding shRNA-resistant wild-type RAC1 or RAC1(P29S), as indicated. The number of EGFP-positive cells was determined by flow cytometry after culture of the cells for the indicated times, and the size of the EGFP-positive fraction relative to that at 3 d was calculated. Data are means+/−SD from three independent experiments for each panel.

FIGS. 6A-6B show the results of RAC1 or NRAS knockdown under low concentrations of FBS. HT1080 cells were transfected with control, RAC1, or NRAS siRNA, as indicated. Each cell fraction was cultured under the presence (FIG. 6A) or absence (FIG. 6B) of 1% (vol/vol) FBS, and the number of cells was counted at the indicated times after the onset of transfection. Data are means+/−SD from three independent experiments.

FIGS. 7A-7B show the distinct effects of siRNAs against RAC1 or NRAS in HT1080 cells. FIG. 7A shows the results of cell cycle profiling examined in HT1080 transfected with control siRNA or siRNA against RAC1 or NRAS, and the relative proportion (%) of G1, S, and G2/M phases is shown for each cell fraction. Data are means+/−SD from three independent experiments. FIG. 7B shows enzymatic activity of CASP3 and CASP7 from the same cell fractions as in FIG. 7A measured with a Caspase-Glo 3/7 Assay kit and shown as a relative activity to that in the cells transfected with control siRNA. Data are means+/−SD from three independent experiments.

FIG. 8 shows the expression of RAC1 resistant to RAC1 siRNA #7. HT1080 cells were infected with mock retrovirus or with virus that expresses siRNA-resistant wild-type RAC1, RAC1(P29S), or RAC1(N92I), followed by transfection with the control siRNA or RAC1 siRNA #7. Expression of RAC1 protein is restored by theintroduction of siRNA-resistant RAC1 cDNAs even in the presence of RAC1 siRNA #7.

FIGS. 9A-9E show the biochemical properties of RAC1 mutants. FIG. 9A shows the results of bacterially expressed and purified proteins of the wild-type, P29S, N92I, or C157Y mutant of RAC1 (5 pmol each) incubated with [³⁵S]GTPgammaS in the presence of 0.8 mM Mg²⁺. The amounts of [³⁵S]GTPgammaS-bound proteins were determined at the indicated times. FIG. 9B shows the results of assays analyzing [³H]GDP dissociation from [³H]GDP-bound RAC1 proteins, which were initiated by the addition of unlabeled GTPgammaS in the presence of 0.8 mM Mg²⁺. The amounts of [³H]GDP-bound proteins were determined at the indicated times. FIG. 9C shows the results of RAC1 proteins preloaded with [gamma³²P]GTP, and then subjected to GTP hydrolysis reactions by the addition of unlabeled GTP in the presence of 0.8 mM Mg²⁺. Pᵢ released from the proteins was isolated and measured at the indicated times. FIG. 9D shows [³⁵S]GTPgammaS dissociation from [³⁵S]GTPgammaS-bound RAC1 proteins initiated by the addition of unlabeled GTPgammaS in the presence of 0.8 mM Mg²⁺. The amounts of [³⁵S]GTPgammaS-bound proteins were determined at the indicated times. FIG. 9E shows a schematic representation of the structure of the GTP-binding pocket of human RAC1 (ID 1mh1 in the Protein Data Bank available on the World Wide Web at pdb.org) with alpha-helices and beta-sheets. The GTP analog, guanosine 5′-(beta,gamma-imido)-triphosphate (GppNp), Mg²⁺, as well as the amino acid residues, D11, P29, N92, and C157, are also shown. In addition, the positions of switch I and switch II regions and the P-loop are indicated.

FIG. 10 shows the structure of the GTP-binding pocket of mutant RAC1 with the P29S, N92I, and C157Y substitutions as predicted by SWISS-MODEL on the basis of the structure of wild-type RAC1 (ID 1e96 in the Protein Data Bank). The alpha-helices, beta-sheets, amino acid residues, D11, S29, Y157, and I92, GppNp, and Mg²⁺, are shown. Loop regions with a low prediction accuracy are also depicted.

FIG. 11 shows the intraprotein interaction of amino acid residues at position 92 of RAC1. In the 3D structure of wild-type RAC1, the side chain of N92 is closest to that of D11, with the amino group of N92 being only 2.8 angstrom distant from the carboxyl group of D11 (top panel). In the predicted structure of RAC1(N92I), however, I92 is still close to D11, but there is no amino-carboxyl interaction (bottom panel).

FIG. 12 shows the endogenous expression of small GTPases in HT1080 cells. The amount of cDNA for RAC1, RAC2, RAC3, NRAS, KRAS, or HRAS was quantified by real-time RT-PCR and demonstrated as a value relative to that for RAC1 (for the RAC family members; left panel) or that for NRAS (for the RAS family members; right panel). Data are means+/−SD from three independent experiments.

FIG. 13 shows siRNA-mediated cell-shape changes. HT1080 cells were transfected with control siRNA or siRNA against RAC1 or NRAS and were examined with a phase-contrast microscope (scale bars, 50 micrometer).

FIGS. 14A-14C show the identification of RAC protein effector molecules. FIG. 14A shows that RAC1 binds to and activates ROCK1 or PAK1 through the Phe-37 or Tyr-40 residue, respectively. FIG. 14B shows the results of anchorage-independent growth in vitro of 3T3 cells infected with recombinant retrovirus used to express wild-type RAC1, RAC1(N92I), RAC1(F37A/N92I) or RAC1(Y40C/N92I). FIG. 14C shows the results of control siRNA or siRNA specific to RAC1, PAK1, ROCK1 or ROCK2 transfected into HT1080 cells after four days of culture.

### DESCRIPTION OF EMBODIMENTS

The present invention is based, at least in part, on the discovery of RAC1 and RAC2 oncogenic mutants having the ability to transform cells. For example, a novel amino acid substitution (N92I) of RAC1 in a sarcoma cell line, HT1080, is described in which the N92I change renders RAC1 constitutively active and highly oncogenic. While HT1080 also carries an NRAS(Q61K) oncoprotein, RAC1(N92I) is the essential growth driver in this cell line, since siRNA-mediated knockdown of RAC1(N92I), but not of NRAS(Q61K), clearly suppressed cell growth. Further screening of RAC1/RAC2/RAC3 mutations among cancer cell lines as well as public databases identified new, transforming mutations for RAC1 and RAC2, such as RAC1(N92I) and RAC2(P29Q). The transforming potential for such new and uncharacterized RAC1 and RAC2 mutants, such as RAC1(P29S), RAC1(C157Y) and RAC2(P29L), were confirmed. For clinical relevance, it was surprisingly determined that that while oncogenic NRAS and RAC1 may be co-present in cancer cells, the latter is essential for cancer growth. Thus, targeting oncogenic RAC proteins with therapeutic agents (e.g., small compounds, RNA-mediated knockdown, or antibodies) are believed to be an effective way to treat cancer harboring the RAC family of oncogenes and targeting downstream mediators for RAC protein is also expected to be beneficial to cancer treatment. In such clinical applications, an accurate diagnosis for the activation of RAC and/or RAC downstream targets are believed to be an important part of the treatment procedure. Indeed, it is demonstrated herein that PAK1, ROCK1, and ROCK2 serine/threonine kinases play important roles in the oncogenic RAC1-/RAC2-mediated oncogenesis and can thereby be useful targets for clinical intervention in subjects having and/or expressing mutant RAC biomarkers described herein. Thus, it has determined herein that oncogenic amino acid substitutions of the RAC family of GTPases exist in human cancer and are useful for identifying, assessing, prognosing, and treating cancer.

In some embodiments, RAC1(N92I) is a superior target for diagnostic, prognostic, and therapeutic intervention. As described herein, fibroblast cells expressing RAC1(N92I) exhibit a significantly increased anchorage-independent growth ability compared to other RAC1 mutants, such as RAC1(P29S), indicating that transforming potential is uneven among various RAC1/RAC2 mutants, and that the RAC1(N92I) mutant is the most potent oncoprotein. This result is important, given that the N92I mutation was discovered in a fibrosarcoma cell line, for which mouse fibroblasts, such as 3T3 cells, likely recapitulates cellular contexts. Indeed, the growth rate of subcutaneous tumors in nude mice is demonstrated herein to be much faster for 3T3 cells expressing RAC1(N92I) than those expressing any other RAC1/RAC2 proteins.

### DEFINITIONS

The articles “a” and “an” refer to one or to more than one (i.e., to at least one) of the grammatical object of the article. By way of example, “an element” means one element or more than one element.

The term “altered amount” of a marker or “altered level” of a marker refers to increased or decreased copy number of the marker and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample.

The “amount” of a marker, e.g., expression or copy number of a marker or minimal common region (MCR), or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker. In some embodiments, the amount of the marker in the subject can be considered “significantly” higher or lower than the normal amount if the amount is 10%, 15%, 20%, 25%, 30%, 35%, 40%, 45%, 50% or more, higher or lower, respectively, than the normal amount of the marker.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “altered subcellular localization” of a marker refers to the mislocalization of the marker within a cell relative to the normal localization within the cell e.g., within a healthy and/or wild-type cell. An indication of normal localization of the marker can be determined through an analysis of subcellular localization motifs known in the field that are harbored by marker polypeptides.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” also includes an “antigen-binding portion” of an antibody (or simply “antibody portion”). The term “antigen-binding portion” refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition” refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “antisense” nucleic acid polypeptide comprises a nucleotide sequence which is complementary to a “sense” nucleic acid encoding a protein, e.g., complementary to the coding strand of a double-stranded cDNA polypeptide, complementary to an mRNA sequence or complementary to the coding strand of a gene. Accordingly, an antisense nucleic acid polypeptide can hydrogen bond to a sense nucleic acid polypeptide.

The term “biochip” refers to a solid substrate comprising an attached probe or plurality of probes of the invention, wherein the probe(s) comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more probes. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder. The probes may be attached to the biochip in a wide variety of ways, as will be appreciated by those in the art. The probes may either be synthesized first, with subsequent attachment to the biochip, or may be directly synthesized on the biochip. The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing. The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics. The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide. The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or preejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit).

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenstroem's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of lymphoma or its subtypes, including, but not limited to, lymphocyte-rich classical Hodgkin lymphoma, mixed cellularity classical Hodgkin lymphoma, lymphocyte-depleted classical Hodgkin lymphoma, nodular sclerosis classical Hodgkin lymphoma, anaplastic large cell lymphoma, diffuse large B-cell lymphomas, MLL⁺ pre B-cell ALL) based upon analysis of markers described herein.

The term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “coding region” refers to regions of a nucleotide sequence comprising codons which are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment. It will be understood by those of skill in the art that such control samples and reference standard expression product levels can be used in combination as controls in the methods of the present invention. In one embodiment, the control may comprise normal or non-cancerous cell/tissue sample. In another embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another embodiment, the control may comprise normal cells, cells from patients treated with combination chemotherapy and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing therapy, or patients having benign cancer. In another embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The term “diagnosing cancer” includes the use of the methods, systems, and code of the present invention to determine the presence or absence of a cancer or subtype thereof in an individual. The term also includes methods, systems, and code for assessing the level of disease activity in an individual.

The term “diagnostic marker” includes markers described herein which are useful in the diagnosis of cancer, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), over-expression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g., a human, afflicted with cancer; (3) its presence or absence in clinical subset of subjects with cancer (e.g., those responding to a particular therapy or those developing resistance). Diagnostic markers also include “surrogate markers,” e.g., markers which are indirect markers of cancer progression, such as increased or sustained expression of PAK1, ROCK1, and/or ROCK2 kinases. Such diagnostic markers may be useful to identify populations of subjects amenable to treatment with modulators of the RAC1 and/or RAC2 mutants and to thereby treat such stratified patient populations.

A molecule is “fixed” or “affixed” to a substrate if it is covalently or non-covalently associated with the substrate such the substrate can be rinsed with a fluid (e.g., standard saline citrate, pH 7.4) without a substantial fraction of the molecule dissociating from the substrate.

The term “gene expression data” or “gene expression level” refers to information regarding the relative or absolute level of expression of a gene or set of genes in a cell or group of cells. The level of expression of a gene may be determined based on the level of RNA, such as mRNA, encoded by the gene. Alternatively, the level of expression may be determined based on the level of a polypeptide or fragment thereof encoded by the gene. Gene expression data may be acquired for an individual cell, or for a group of cells such as a tumor or biopsy sample. Gene expression data and gene expression levels can be stored on computer readable media, e.g., the computer readable medium used in conjunction with a microarray or chip reading device. Such gene expression data can be manipulated to generate gene expression signatures.

The term “gene expression signature” or “signature” refers to a group of coordinately expressed genes. The genes making up this signature may be expressed in a specific cell lineage, stage of differentiation, or during a particular biological response. The genes can reflect biological aspects of the tumors in which they are expressed, such as the cell of origin of the cancer, the nature of the non-malignant cells in the biopsy, and the oncogenic mechanisms responsible for the cancer.

The term “homologous” refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term.

The term “humanized antibody” is intended to include antibodies made by a non-human cell having variable and constant regions which have been altered to more closely resemble antibodies that would be made by a human cell, for example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody” also includes antibodies in which CDR sequences derived from the germline of another mammalian species, such as a mouse, have been grafted onto human framework sequences.

The term “inhibit” includes the decrease, limitation, or blockage, of, for example a particular action, function, or interaction. For example, cancer is “inhibited” if at least one symptom of the cancer, such as hyperproliferative growth, is alleviated, terminated, slowed, or prevented. Cancer is also “inhibited” if recurrence or metastasis of the cancer is reduced, slowed, delayed, or prevented.

The term “inhibitor,” when used in conjunction with decreasing the action, function, or interaction of a biomarker of the present invention, may be any as along as it inhibits a desired target nucleic acid or protein encoded therefrom being present in an activated state (e.g., constitutive, oncogenic, and/or phosphorylated state). Non-limiting examples, include nucleic acid inhibitors, small molecule inhibitors, blocking antibodies, a peptide that competitively interacts with a target protein, a substrate for an enzyme that phosphorylates the target protein, a phosphatase that acts on a target protein, and the like. The phosphorylation site and/or physiologically active domain of RAC1, RAC2, RAC3, PAK1, ROCK1, and ROCK2, for example, are known, and those of ordinary skill in the art can easily design or synthesize an agent to inhibit the target. Confirmation of the expression inhibitory activity, activation inhibitory activity, destabilization activity and the like of a target can be measured according to known methods. For example, PAK1 inhibitory activity can be measured, for example, by modifying the measurement experiment of LIMK activation by PAK1 described in Nat Cell Biol. (1999) 1, 253-259, performing the experiment using a test substance, and considering the activation inhibitory rate. Similarly, ROCK1/2 inhibitory activity can be measured, for example, by the method described in Biochem. J. (2000) 351, 95-105, performing the experiment using a test substance, and considering the activation inhibitory rate (e.g., by measuring the phosphorylation of the proteins, Western blotting or ELISA using a phosphorylation antibody that specifically reacts with those proteins). Exemplary PAK1 inhibitors are well-known in the art and include small molecule inhibitors such as those described in U.S. Pat. Publs. 2013-0116263, 2012-0270866, and 2005-0037965; all of which are incorporated by reference. Exemplary ROCK1/2 inhibitors include Y-27632 and fasudil, which bind to the kinase domain to inhibit its enzymatic activity in an ATP-competitive mechanism. Negative regulators of ROCK1/2 activation include small GTP-binding proteins such as Gem, RhoE, and Rad, which can attenuate ROCK1/2 activity. Autoinhibitory activity of ROCK is demonstrated upon interaction of the carboxyl terminus with the kinase domain to reduce kinase activity. Additional ROCK1/2 inhibitors are described in WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227; all of which are incorporated by reference. In some of these cases, motifs in the inhibitors include an indazole core; a 2-aminopyridine/pyrimidine core; a 9-deazaguanine deriviative; benzamide-comprising; aminofurazan-comprising; and/or a combination thereof.

The term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules. Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

An “isolated antibody” is intended to refer to an antibody that is substantially free of other antibodies having different antigenic specificities. Moreover, an isolated antibody may be substantially free of other cellular material and/or chemicals.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g., a package or container) comprising at least one reagent, e.g., a probe, for specifically detecting or modulating the expression of a marker of the invention. The kit may be promoted, distributed, or sold as a unit for performing the methods of the present invention.

A “marker” or “biomarker” includes a nucleic acid or polypeptide whose altered level of expression in a tissue or cell from its expression level in a control (e.g., normal or healthy tissue or cell) is associated with a disease state, such as a cancer or subtype thereof. A “marker nucleic acid” is a nucleic acid (e.g., mRNA, cDNA, mature miRNA, pre-miRNA, pri-miRNA, miRNA*, anti-miRNA, or a miRNA binding site, or a variant thereof and other classes of small RNAs known to a skilled artisan) encoded by or corresponding to a marker of the invention. Such marker nucleic acids include DNA (e.g., cDNA) comprising the entire or a partial sequence of any of the nucleic acid sequences set forth herein or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” includes a protein encoded by or corresponding to a marker of the invention. A marker protein comprises the entire or a partial sequence of any of the sequences set forth in herein. The terms “protein” and “polypeptide” are used interchangeably. In some embodiments, specific combinations of biomarkers are preferred. For example, a combination or subgroup of one or more of the biomarkers selected from the group consisting of RAC1 and/or RAC2 mutants desribed herein.

The term “modulate” includes up-regulation and down-regulation, e.g., enhancing or inhibiting a response.

The “normal” or “control” level of expression of a marker is the level of expression of the marker in cells of a subject, e.g., a human patient, not afflicted with a cancer. An “over-expression” or “significantly higher level of expression” of a marker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., lymphoid cancers, such as leukemia), development of one or more clinical factors, development of intestinal cancer, or recovery from the disease.

The term “response to cancer therapy” or “outcome of cancer therapy” relates to any response of the hyperproliferative disorder (e.g., cancer) to a cancer therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection for solid cancers. Responses may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to copy number, level of expression, level of activity, etc. of one or more biomarkers listed or described herein or the Examples that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom the measurement values are known. In certain embodiments, the same doses of cancer therapeutic agents are administered to each subject. In related embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker threshold values that correlate to outcome of a cancer therapy can be determined using methods such as those described in the Examples section. Outcomes can also be measured in terms of a “hazard ratio” (the ratio of death rates for one patient group to another; provides likelihood of death at a certain time point), “overall survival” (OS), and/or “progression free survival.” In certain embodiments, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. Thus, the present invention further provides methods for making a treatment decision for a cancer patient, comprising carrying out the methods for prognosing a cancer patient according to the different aspects and embodiments of the present invention, and then weighing the results in light of other known clinical and pathological risk factors, in determining a course of treatment for the cancer patient. For example, a cancer patient that is shown by the methods of the invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., chemotherapeutic or radiation therapy. In some embodiments, normal cells are not affected to an extent that causes the normal cells to be unduly injured by the cancer therapy (e.g., chemotherapy or radiation therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “synergistic effect” refers to the combined effect of two or more anti-cancer agents or chemotherapy drugs can be greater than the sum of the separate effects of the anticancer agents or chemotherapy drugs alone.

The term “subject” refers to any healthy animal, mammal or human, or any animal, mammal or human afflicted with a condition of interest (e.g., cancer). The term “subject” is interchangeable with “patient.”

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

The term “substantially pure cell population” refers to a population of cells having a specified cell marker characteristic and differentiation potential that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

A “transcribed polynucleotide” or “nucleotide transcript” is a polynucleotide (e.g., an mRNA, hnRNA, cDNA, mature miRNA, pre-miRNA, pri-miRNA, miRNA*, anti-miRNA, or a miRNA binding site, or a variant thereof or an analog of such RNA or cDNA) which is complementary to or homologous with all or a portion of a mature mRNA made by transcription of a marker of the invention and normal post-transcriptional processing (e.g., splicing), if any, of the RNA transcript, and reverse transcription of the RNA transcript.

The term “vector” refers to a nucleic acid capable of transporting another nucleic acid to which it has been operably linked. “Operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors.” In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.). An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to those described above for use as fusion partners.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) and preferably, the average expression level or copy number of the marker in several control samples.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for a fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Finally, nucleic acid and amino acid sequence information for the general sequence and structures of the biomarkers of the present invention are well known in the art and readily available on publicly available databases, such as the National Center for Biotechnology Information (NCBI). Exemplary nucleic acid and amino acid sequences derived from publicly available sequence databases are provided below.

In some embodiments, the methods and compositions of the present disclosure can use well-known RAC1, RAC2, and/or RAC3 gene sequences or fragments thereof, as well as gene products of the RAC1, RAC2, and/or RAC3 gene sequences, e.g., polypeptides and antibodies which specifically bind to such gene products, or fragments thereof, as starting points for generating mutants. Sequences, splice variants, and structures of RAC1, RAC2, and/or RAC3 gene sequences and gene products have been described in the art. See, for example, the Gene Cards.com website available on the World Wide Web at genecards.org/cgi-bin/carddisp.pl?gene=RAC1, genecards.org/cgi-bin/carddisp.pl?gene=RAC2, and genecards.org/cgi-bin/carddisp.pl?gene=RAC3.

RAC1, RAC2, and/or RAC3 gene sequences and gene product sequences, as well as those for RAC effectors, such as PAK1, ROCK1, and ROCK2, are known from many species.

At least two splice variants encoding two human RAC1 isoforms exist. The sequence of human RAC1 transcript variant 1 is the canonical sequence, all positional information described with respect to the remaining isoforms are determined from this sequence, and the sequences are available to the public at the GenBank database under NM_{—}006908.4 and NP_{—}008839.2. The sequences of human RAC1 transcript variant 2 can be found under NM_{—}018890.3 and NP_{—}691485.1 and the encoded protein includes the alternatively spliced 57 bp region (exon 3b) that is missing in transcript variant RAC1. Nucleic acid and polypeptide sequences of RAC1 orthologs in organisms other than humans are well known and include, for example, mouse RAC1 (NM_{—}009007.2 and NP_{—}033033.1), rat RAC1 (NM_{—}134366.1 and NP_{—}599193.1), chicken RAC1 (NM_{—}205017.1 and NP_{—}990348.1), zebrafish RAC1 (NM_{—}199771.1 and NP_{—}956065.1), cow RAC1 (NM_{—}174163.2 and NP_{—}776588.1), and dog RAC1 (NM_{—}001003274.2 and NP_{—}001003274.1).

The nucleic acid and amino acid sequences of a representative human RAC2 biomarker is available to the public at the GenBank database under NM_{—}002872.3 and NP_{—}002863.1. Nucleic acid and polypeptide sequences of RAC2 orthologs in organisms other than humans are well known and include, for example, mouse RAC2 (NM_{—}009008.3 and NP_{—}033034.1), rat RAC2 (NM_{—}001008384.1 and NP_{—}001008385.1), chimpanzee RAC2 (XM_{—}001145815.3 and XP_{—}001145815.3), monkey RAC2 (XM_{—}001086228.2 and XP_{—}001086228.1), dog RAC2 (XM_{—}538392.4 and XP_{—}538392.4), cow RAC2 (NM_{—}175792.2 and NP_{—}786986.1), chicken RAC2 (NM_{—}001201452.1 and NP_{—}001188381.1), and zebrafish RAC2 (NM_{—}001002061.1 and NP_{—}001002061.1).

The nucleic acid and amino acid sequences of a representative human RAC3 biomarker is available to the public at the GenBank database under NM_{—}005052.2 and NP_{—}005043.1. Nucleic acid and polypeptide sequences of RAC3 orthologs in organisms other than humans are well known and include, for example, mouse RAC3 (NM_{—}133223.4 and NP_{—}573486.1), monkey RAC3 (XM_{—}001113336.2 and XP_{—}00111336.2), cow RAC3 (NM_{—}001099179.1 and NP_{—}001092649.1), and chicken RAC3 (NM_{—}205016.1 and NP_{—}990347.1).

At least two splice variants encoding two human p21 protein (Cdc42/Rac)-activated kinase 1 (PAK1) isoforms exist. The sequence of human PAK1 transcript variant 1 is the canonical sequence, all positional information described with respect to the remaining isoforms are determined from this sequence, and the sequences are available to the public at the GenBank database under NM_{—}001128620.1 and NP_{—}001122092.1. The sequences of human PAK1 transcript variant 2 can be found under NM_{—}002576.4 and NP_{—}002567.3. Human PAK1 transcript variant 2 lacks a coding exon in the 3′ region resulting in an isoform that has a shorter and distinct C-terminus. Nucleic acid and polypeptide sequences of PAK1 orthologs in organisms other than humans are well known and include, for example, mouse PAK1 (NM_{—}011035.2 and NP_{—}035165.2), rat PAK1 (NM_{—}017198.1 and NP_{—}058894.1), chimpanzee PAK1 (XM_{—}508657.4 and XP_{—}508657.3), monkey PAK1 (XM_{—}001090310.2, XP_{—}001090310.1, XM_{—}001090423.2, and XP_{—}001090423.2), dog PAK1 (XM_{—}844558.2 and XP_{—}849651.1), cow PAK1 (NM_{—}001076898.1 and NP_{—}001070366.1), chicken PAK1 (NM_{—}001162372.2 and NP_{—}001155844.1), and zebrafish PAK1 (NM_{—}201328.1 and NP_{—}958485.1).

The nucleic acid and amino acid sequences of a representative human Rho-associated, coiled-coil containing protein kinase 1 (ROCK1) biomarker is available to the public at the GenBank database under NM_{—}005406.2 and NP_{—}005397.1. Nucleic acid and polypeptide sequences of ROCK1 orthologs in organisms other than humans are well known and include, for example, mouse ROCK1 (NM_{—}009071.2 and NP_{—}033097.1), rat ROCK1 (NM_{—}031098.1 and NP_{—}112360.1), chimpanzee ROCK1 (XM_{—}001151982.1 and XP_{—}001151982.1), monkey ROCK1 (NM_{—}001261134.1 and NP_{—}001248063.1), dog ROCK1 (XM_{—}537305.4 and XP_{—}537305.3), cow ROCK1 (XM_{—}003583969.1 and XP_{—}003584017.1), and chicken ROCK1 (NM_{—}001199448.1 and NP_{—}001186377.1).

The nucleic acid and amino acid sequences of a representative human Rho-associated, coiled-coil containing protein kinase 2 (ROCK2) biomarker is available to the public at the GenBank database under NM_{—}004850.3 and NP_{—}004841.2. Nucleic acid and polypeptide sequences of ROCK2 orthologs in organisms other than humans are well known and include, for example, mouse ROCK2 (NM_{—}009072.2 and NP_{—}033098.2), rat ROCK2 (NM_{—}013022.2 and NP_{—}037154.2), chimpanzee ROCK2 (XM_{—}525689.3 and XP_{—}525689.3), monkey ROCK2 (XM_{—}001096931.2 and XP_{—}001096931.2), dog ROCK2 (XM_{—}540083.3 and XP_{—}540083.3), cow ROCK2 (NM_{—}174452.2 and NP_{—}776877.1), and zebrafish ROCK2 (NM_{—}174863.2 and NP_{—}777288.1).

The following are representative sequences useful for generating mutant RAC biomarkers of the present invention. Such mutant RAC biomarkers may be from any species of origin. In one embodiment, the mutant RAC marker is from a mammalian species (e.g., of human or murine origin). A “mutant” or “variant” polypeptide contains at least one amino acid sequence alteration as compared to the amino acid sequence of the corresponding wild-type polypeptide. An amino acid sequence alteration can be, for example, a substitution, a deletion, or an insertion of one or more amino acids

| RAC1 cDNA nucleic acid sequence (GenBank Acc. Num. NM_006908.4; | |
|---|---|
| SEQ ID NO: 1): | |
| 1 | gggaggccgg atgtgagtgg agcggccatt tcctgtttct ctgcagtttt cctcagcttt |
| 61 | gggtggtggc cgctgccggg catcggcttc cagtccgcgg agggcgaggc ggcgtggaca |
| 121 | gcggccccgg cacccagcgc cccgccgccc gcaagccgcg cgcccgtccg ccgcgccccg |
| 181 | agcccgccgc ttcctatctc agcgccctgc cgccgccgcc gcggcccagc gagcggccct |
| 241 | gatgcaggcc atcaagtgtg tggtggtggg agacggagct gtaggtaaaa cttgcctact |
| 301 | gatcagttac acaaccaatg catttcctgg agaatatatc cctactgtct ttgacaatta |
| 361 | ttctgccaat gttatggtag atggaaaacc ggtgaatctg ggcttatggg atacagctgg |
| 421 | acaagaagat tatgacagat tacgccccct atcctatccg caaacagatg tgttcttaat |
| 481 | ttgcttttcc cttgtgagtc ctgcatcatt tgaaaatgtc cgtgcaaagt ggtatcctga |
| 541 | ggtgcggcac cactgtccca acactcccat catcctagtg ggaactaaac ttgatcttag |
| 601 | ggatgataaa gacacgatcg agaaactgaa ggagaagaag ctgactccca tcacctatcc |
| 661 | gcagggtcta gccatggcta aggagattgg tgctgtaaaa tacctggagt gctcggcgct |
| 721 | cacacagcga ggcctcaaga cagtgtttga cgaagcgatc cgagcagtcc tctgcccgcc |
| 781 | tcccgtgaag aagaggaaga gaaaatgcct gctgttgtaa atgtctcagc ccctcgttct |
| 841 | tggtcctgtc ccttggaacc tttgtacgct ttgctcaaaa aaaaacaaaa aaaaaaaaca |
| 901 | aaaaaaaaaa acaacggtgg agccttcgca ctcaatgcca actttttgtt acagattaat |
| 961 | ttttccataa aaccattttt tgaaccaatc agtaatttta aggttttgtt tgttctaaat |
| 1021 | gtaagagttc agactcacat tctattaaaa tttagcccta aaatgacaag ccttcttaaa |
| 1081 | gccttatttt tcaaaagcgc cccccccatt cttgttcaga ttaagagttg ccaaaatacc |
| 1141 | ttctgaacta cactgcattg ttgtgccgag aacaccgagc actgaacttt gcaaagacct |
| 1201 | tcgtctttga gaagacggta gcttctgcag ttaggaggtg cagacacttg ctctcctatg |
| 1261 | tagttctcag atgcgtaaag cagaacagcc tcccgaatga agcgttgcca ttgaactcac |
| 1321 | cagtgagtta gcagcacgtg ttcccgacat aacattgtac tgtaatggag tgagcgtagc |
| 1381 | agctcagctc tttggatcag tctttgtgat ttcatagcga gttttctgac cagcttttgc |
| 1441 | ggagattttg aacagaactg ctatttcctc taatgaagaa ttctgtttag ctgtgggtgt |
| 1501 | gccgggtggg gtgtgtgtga tcaaaggaca aagacagtat tttgacaaaa tacgaagtgg |
| 1561 | agatttacac tacattgtac aaggaatgaa agtgtcacgg gtaaaaactc taaaaggtta |
| 1621 | atttctgtca aatgcagtag atgatgaaag aaaggttggt attatcagga aatgttttct |
| 1681 | taagcttttc ctttctctta cacctgccat gcctccccaa attgggcatt taattcatct |
| 1741 | ttaaactggt tgttctgtta gtcgctaact tagtaagtgc ttttcttata gaaccccttc |
| 1801 | tgactgagca atatgcctcc ttgtattata aaatctttct gataatgcat tagaaggttt |
| 1861 | ttttgtcgat tagtaaaagt gctttccatg ttactttatt cagagctaat aagtgctttc |
| 1921 | cttagttttc tagtaactag gtgtaaaaat catgtgttgc agctttatag tttttaaaat |
| 1981 | attttagata attcttaaac tatgaacctt cttaacatca ctgtcttgcc agattaccga |
| 2041 | cactgtcact tgaccaatac tgaccctctt tacctcgccc acgcggacac acgcctcctg |
| 2101 | tagtcgcttt gcctattgat gttcctttgg gtctgtgagg ttctgtaaac tgtgctagtg |
| 2161 | ctgacgatgt tctgtacaac ttaactcact ggcgagaata cagcgtggga cccttcagcc |
| 2221 | actacaacag aattttttaa attgacagtt gcagaattgt ggagtgtttt tacattgatc |
| 2281 | ttttgctaat gcaattagca ttatgttttg catgtatgac ttaataaatc cttgaatcat |
| 2341 | a |
| RAC1 amino acid sequence (GenBank Acc. Num. NP_008839.2; SEQ ID NO: 2): | |
| 1 | mqaikcvvvg dgavgktcll isyttnafpg eyiptvfdny sanvmvdgkp vnlglwdtag |
| 61 | qedydrlrpl sypqtdvfli cfslvspasf envrakwype vrhhcpntpi ilvgtkldlr |
| 121 | ddkdtieklk ekkltpityp qglamakeig avkylecsal tqrglktvfd eairavlcpp |
| 181 | pvkkrkrkcl ll |
| RAC1b cDNA nucleic acid sequence (GenBank Acc. Num. NM_018890.3; | |
| SEQ ID NO: 3): | |
| 1 | gggaggccgg atgtgagtgg agcggccatt tcctgtttct ctgcagtttt cctcagcttt |
| 61 | gggtggtggc cgctgccggg catcggcttc cagtccgcgg agggcgaggc ggcgtggaca |
| 121 | gcggccccgg cacccagcgc cccgccgccc gcaagccgcg cgcccgtccg ccgcgccccg |
| 181 | agcccgccgc ttcctatctc agcgccctgc cgccgccgcc gcggcccagc gagcggccct |
| 241 | gatgcaggcc atcaagtgtg tggtggtggg agacggagct gtaggtaaaa cttgcctact |
| 301 | gatcagttac acaaccaatg catttcctgg agaatatatc cctactgtct ttgacaatta |
| 361 | ttctgccaat gttatggtag atggaaaacc ggtgaatctg ggcttatggg atacagctgg |
| 421 | acaagaagat tatgacagat tacgccccct atcctatccg caaacagttg gagaaacgta |
| 481 | cggtaaggat ataacctccc ggggcaaaga caagccgatt gccgatgtgt tcttaatttg |
| 541 | cttttccctt gtgagtcctg catcatttga aaatgtccgt gcaaagtggt atcctgaggt |
| 601 | gcggcaccac tgtcccaaca ctcccatcat cctagtggga actaaacttg atcttaggga |
| 661 | tgataaagac acgatcgaga aactgaagga gaagaagctg actcccatca cctatccgca |
| 721 | gggtctagcc atggctaagg agattggtgc tgtaaaatac ctggagtgct cggcgctcac |
| 781 | acagcgaggc ctcaagacag tgtttgacga agcgatccga gcagtcctct gcccgcctcc |
| 841 | cgtgaagaag aggaagagaa aatgcctgct gttgtaaatg tctcagcccc tcgttcttgg |
| 901 | tcctgtccct tggaaccttt gtacgctttg etcaaaaaaa aacaaaaaaa aaaaacaaaa |
| 961 | aaaaaaaaca acggtggagc cttcgcactc aatgccaact ttttgttaca gattaatttt |
| 1021 | tccataaaac cattttttga accaatcagt aattttaagg ttttgtttgt tctaaatgta |
| 1081 | agagttcaga ctcacattct attaaaattt agccctaaaa tgacaagcct tcttaaagcc |
| 1141 | ttatttttca aaagcgcccc ccccattctt gttcagatta agagttgcca aaataccttc |
| 1201 | tgaactacac tgcattgttg tgccgagaac accgagcact gaactttgca aagaccttcg |
| 1261 | tctttgagaa gacggtagct tctgcagtta ggaggtgcag acacttgctc tcctatgtag |
| 1321 | ttctcagatg cgtaaagcag aacagcctcc cgaatgaagc gttgccattg aactcaccag |
| 1381 | tgagttagca gcacgtgttc ccgacataac attgtactgt aatggagtga gcgtagcagc |
| 1441 | tcagctcttt ggatcagtct ttgtgatttc atagcgagtt ttctgaccag cttttgcgga |
| 1501 | gattttgaac agaactgcta tttcctctaa tgaagaattc tgtttagctg tgggtgtgcc |
| 1561 | gggtggggtg tgtgtgatca aaggacaaag acagtatttt gacaaaatac gaagtggaga |
| 1621 | tttacactac attgtacaag gaatgaaagt gtcacgggta aaaactctaa aaggttaatt |
| 1681 | tctgtcaaat gcagtagatg atgaaagaaa ggttggtatt atcaggaaat gttttcttaa |
| 1741 | gcttttcctt tctcttacac ctgccatgcc tccccaaatt gggcatttaa ttcatcttta |
| 1801 | aactggttgt tctgttagtc gctaacttag taagtgcttt tcttatagaa ccccttctga |
| 1861 | ctgagcaata tgcctccttg tattataaaa tctttctgat aatgcattag aaggtttttt |
| 1921 | tgtcgattag taaaagtgct ttccatgtta ctttattcag agctaataag tgctttcctt |
| 1981 | agttttctag taactaggtg taaaaatcat gtgttgcagc tttatagttt ttaaaatatt |
| 2041 | ttagataatt cttaaactat gaaccttctt aacatcactg tcttgccaga ttaccgacac |
| 2101 | tgtcacttga ccaatactga ccctctttac ctcgcccacg cggacacacg cctcctgtag |
| 2161 | tcgctttgcc tattgatgtt cctttgggtc tgtgaggttc tgtaaactgt gctagtgctg |
| 2221 | acgatgttct gtacaactta actcactggc gagaatacag cgtgggaccc ttcagccact |
| 2281 | acaacagaat tttttaaatt gacagttgca gaattgtgga gtgtttttac attgatcttt |
| 2341 | tgctaatgca attagcatta tgttttgcat gtatgactta ataaatcctt gaatcata |
| RAC1b amino acid sequence (GenBank Acc. Num. NP_061485.1; SEQ ID No: 4): | |
| 1 | mqaikcvvvg dgavgktcll isyttnafpg eyiptvfdny sanvmvdgkp vnlglwdtag |
| 61 | qedydrlrpl sypqtvgety gkditsrgkd kpiadvflic fslvspasfe nvrakwypev |
| 121 | rhhcpntpii lvgtkldlrd dkdtieklke kkltpitypq glamakeiga vkylecsalt |
| 181 | qrglktvfde airavlcppp vkkrkrkcll l |
| RAC2 cDNA nucleic acid sequence (GenBank Acc. Num. NM_002872.3; | |
| SEQ ID NO: 5): | |
| 1 | tgccccacca ccgctgctcc tcagcaggcg cctcaccagc ctccacaccc cttgcgcccg |
| 61 | cagaaacgcg cctggccctg agctgtcacc accgacactc tccaggctcc ggacacgatg |
| 121 | caggccatca agtgtgtggt ggtgggagat ggggccgtgg gcaagacctg ccttctcatc |
| 181 | agctacacca ccaacgcctt tcccggagag tacatcccca ccgtgtttga caactattca |
| 241 | gccaatgtga tggtggacag caagccagtg aacctggggc tgtgggacac tgctgggcag |
| 301 | gaggactacg accgtctccg gccgctctcc tatccacaga cggacgtctt cctcatctgc |
| 361 | ttctccctcg tcagcccagc ctcttatgag aacgtccgcg ccaagtggtt cccagaagtg |
| 421 | cggcaccact gccccagcac acccatcatc ctggtgggca ccaagctgga cctgcgggac |
| 481 | gacaaggaca ccatcgagaa actgaaggag aagaagctgg ctcccatcac ctacccgcag |
| 541 | ggcctggcac tggccaagga gattgactcg gtgaaatacc tggagtgctc agctctcacc |
| 601 | cagagaggcc tgaaaaccgt gttcgacgag gccatccggg ccgtgctgtg ccctcagccc |
| 661 | acgcggcage agaagcgcgc ctgcagcctc ctctaggggt tgcaccccag cgctcccacc |
| 721 | tagatgggtc tgatcctcca ggatccccac ccaaagcctg atggcacccc ggctggccat |
| 781 | gctgtcccct ccctgtggcg tttcttagca gatggctgca gagcttcgtt gatggtcttt |
| 841 | tctgtactgg aggcctcctg aggccaggaa cgtgcaaatt tgcaggtgct gcatcccaag |
| 901 | cccctcatgc tcctgccttc ctgagggcca gaggggagcc ccaggaccca ttaagccacc |
| 961 | cccgtgttcc tgccgtcagt gccaactgcc gcatgtggaa gcatctaccc gttcactcca |
| 1021 | gtcccacccc acgcctgact cccctctgga aactgcaggc cagatggttg ctgccacaac |
| 1081 | ttgtgtacct tcagggatgg ggctcttact ccctcctgag gccagctgct ctaatatcga |
| 1141 | tggtcctgct tgccagagag ttcctctacc cagcaaaaat gagtgtctca gaagtgtgct |
| 1201 | cctctggcct cagttctcct cttttggaac aacataaaac aaatttaatt ttctacgcct |
| 1261 | ctggggatat ctgctcagcc aatggaaaat ctgggttcaa ccagcccctg ccatttctta |
| 1321 | agactttctg ctgcactcac aggatcctga gctgcactta cctgtgagag tcttcaaact |
| 1381 | tttaaacctt gccagtcagg acttttgcta ttgcaaatag aaaacccaac tcaacctgct |
| 1441 | taagcagaaa ataaatttat tgattcaaaa aaaaaaaaaa aaaaaaaaaa aaaaaaaaaa |
| 1501 | aaaaaaaaaa aaaaaa |
| RAC2 amino acid sequence (GenBank Acc. Num. NP_002863.1; SEQ ID NO: 6): | |
| 1 | mqaikcvvvg dgavgktcll isyttnafpg eyiptvfdny sanvmvdskp vnlglwdtag |
| 61 | qedydrlrpl sypqtdvfli cfslvspasy envrakwfpe vrhhcpstpi ilvgtkldlr |
| 121 | ddkdtieklk ekklapityp qglalakeid svkylecsal tqrglktvfd eairavlcpq |
| 181 | ptrqqkracs ll |
| RAC3 cDNA nucleic acid sequence (GenBank Acc. Num. NM_005052.2; | |
| SEQ ID NO: 7): | |
| 1 | tgtctccggc cgatcgctcg gcgctcgggt ccgcggccgc tgcggcgccg ggcatttctc |
| 61 | cgcagctcgg ctcgcggccg cgcccgccgc cgcccggccc gcgcccatgc aggccatcaa |
| 121 | gtgcgtggtg gteggcgacg gcgccgtggg gaagacatgc ttgctgatca gctacacgac |
| 181 | caacgccttc cccggagagt acatccccac cgtttttgac aactactctg ccaacgtgat |
| 241 | ggtggacggg aaaccagtca acttggggct gtgggacaca gcgggtcagg aggactacga |
| 301 | tcggctgcgg ccactctcct acccccaaac tgacgtcttt ctgatctgct tctctctggt |
| 361 | gagcccggcc tccttcgaga atgttcgtgc caagtggtac ccggaggtgc ggcaccactg |
| 421 | cccccacacg cccatcctcc tggtgggcac caagctggac ctccgcgacg acaaggacac |
| 481 | cattgagcgg ctgcgggaca agaagctggc acccatcacc tacccacagg gcctggccat |
| 541 | ggcccgggag attggctctg tgaaatacct ggagtgctca gccctgaccc agcggggcct |
| 601 | gaagacagtg tttgacgagg cgatccgcgc ggtgctctgc ccgcccccag tgaagaagcc |
| 661 | ggggaagaag tgcaccgtct tctagagccc tggcccaccc gagcctgagg gctggcgggg |
| 721 | agcagccctg gacgtgtccg ctgttgtgtt gagacgtgtg gtgtccctga gtcggctgtg |
| 781 | gggagcggtg ggggtgggcc ggggggaagc atggggatga ggctgggtgg caggatcctg |
| 841 | tcctctctgc cgcctcattc tggggtgtgg ctccagcctt ccctggcccc cgccggaggc |
| 901 | cgggagggag cagggtctcc ctcagggctg caggggcagg tgcagggaag ccccaggatg |
| 961 | ggcttccctg gagggggagg gtggggggga gttctgttcc ttgtgccccg aggtggggca |
| 1021 | gccccttctc attttataca ataaacattc tccacctaca aaaaaaaaaa aaaaaaa |
| RAC3 amino acid sequence (GenBank Acc. Num. NP_005043.1; SEQ ID NO: 8): | |
| 1 | mqaikcvvvg dgavgktcll isyttnafpg eyiptvfdny sanvmvdgkp vnlglwdtag |
| 61 | qedydrlrpl sypqtdvfli cfslvspasf envrakwype vrhhcphtpi llvgtkldlr |
| 121 | ddkdtierlr dkklapityp qglamareig svkylecsal tqrglktvfd eairavlcpp |
| 181 | pvkkpgkkct vf |
| PAK1 transcript variant 1 cDNA nucleic acid sequence (GenBank Acc. Num. | |
| NM_001128620.1; SEQ ID NO: 9): | |
| 1 | atgtcaaata acggcctaga cattcaagac aaacccccag cccctccgat gagaaatacc |
| 61 | agcactatga ttggagccgg cagcaaagat gctggaaccc taaaccatgg ttctaaacct |
| 121 | ctgcctccaa acccagagga gaagaaaaag aaggaccgat tttaccgatc cattttacct |
| 181 | ggagataaaa caaataaaaa gaaagagaaa gagcggccag agatttctct cccttcagat |
| 241 | tttgaacaca caattcatgt cggttttgat gctgtcacag gggagtttac gggaatgcca |
| 301 | gagcagtggg cccgcttgct tcagacatca aatatcacta agtcggagca gaagaaaaac |
| 361 | ccgcaggctg ttctggatgt gttggagttt tacaactcga agaagacatc caacagccag |
| 421 | aaatacatga gctttacaga taagtcagct gaggattaca attcttctaa tgccttgaat |
| 481 | gtgaaggctg tgtctgagac tcctgcagtg ccaccagttt cagaagatga ggatgatgat |
| 541 | gatgatgatg ctaccccacc accagtgatt gctccacgcc cagagcacac aaaatctgta |
| 601 | tacacacggt ctgtgattga accacttcct gtcactccaa ctcgggacgt ggctacatct |
| 661 | cccatttcac ctactgaaaa taacaccact ccaccagatg ctttgacccg gaatactgag |
| 721 | aagcagaaga agaagcctaa aatgtctgat gaggagatct tggagaaatt acgaagcata |
| 781 | gtgagtgtgg gcgatcctaa gaagaaatat acacggtttg agaagattgg acaaggtgct |
| 841 | tcaggcaccg tgtacacagc aatggatgtg gccacaggac aggaggtggc cattaagcag |
| 901 | atgaatcttc agcagcagcc caagaaagag ctgattatta atgagatcct ggtcatgagg |
| 961 | gaaaacaaga acccaaacat tgtgaattac ttggacagtt acctcgtggg agatgagctg |
| 1021 | tgggttgtta tggaatactt ggctggaggc tccttgacag atgtggtgac agaaacttgc |
| 1081 | atggatgaag gccaaattgc agctgtgtgc cgtgagtgtc tgcaggctct ggagttcttg |
| 1141 | cattcgaacc aggtcattca cagagacatc aagagtgaca atattctgtt gggaatggat |
| 1201 | ggctctgtca agctaactga ctttggattc tgtgcacaga taaccccaga gcagagcaaa |
| 1261 | cggagcacca tggtaggaac cccatactgg atggcaccag aggttgtgac acgaaaggcc |
| 1321 | tatgggccca aggttgacat ctggtccctg ggcatcatgg ccatcgaaat gattgaaggg |
| 1381 | gagcctccat acctcaatga aaaccctctg agagccttgt acctcattgc caccaatggg |
| 1441 | accccagaac ttcagaaccc agagaagctg tcagctatct tccgggactt tctgaaccgc |
| 1501 | tgtctcgaga tggatgtgga gaagagaggt tcagctaaag agctgctaca ggtgagaaaa |
| 1561 | ctgaggtttc aagtgtttag taacttttcc atgatagctg catcaattcc tgaagattgc |
| 1621 | caagcccctc tccagcctca ctccactgat tgctgcagct aa |
| PAK1 isoform 1 amino acid sequence (GenBank Acc. Num. NP_001122092.1; | |
| SEQ ID NO: 10): | |
| 1 | msnngldiqd kppappmrnt stmigagskd agtlnhgskp lppnpeekkk kdrfyrsilp |
| 61 | gdktnkkkek erpeislpsd fehtihvgfd avtgeftgmp eqwarllqts nitkseqkkn |
| 121 | pqavldvlef ynskktsnsq kymsftdksa edynssnaln vkavsetpav ppvsededdd |
| 181 | dddatpppvi aprpehtksv ytrsvieplp vtptrdvats pisptenntt ppdaltrnte |
| 241 | kqkkkpkmsd eeileklrsi vsvgdpkkky trfekigqga sgtvytamdv atgqevaikq |
| 301 | mnlqqqpkke liineilvmr enknpnivny ldsylvgdel wvvmeylagg sltdvvtetc |
| 361 | mdegqiaavc reclqalefl hsnqvihrdi ksdnillgmd gsvkltdfgf caqitpeqsk |
| 421 | rstmvgtpyw mapevvtrka ygpkvdiwsl gimaiemieg eppylnenpl ralyliatng |
| 481 | tpelqnpekl saifrdflnr clemdvekrg sakellqvrk lrfqvfsnfs miaasipedc |
| 541 | qaplqphstd ccs |
| PAK1 transcript variant 2 cDNA nucleic acid sequence (GenBank Acc. Num. | |
| NM_002576.4; SEQ ID NO: 11): | |
| 1 | atgtcaaata acggcctaga cattcaagac aaacccccag cccctccgat gagaaatacc |
| 61 | agcactatga ttggagccgg cagcaaagat gctggaaccc taaaccatgg ttctaaacct |
| 121 | ctgcctccaa acccagagga gaagaaaaag aaggaccgat tttaccgatc cattttacct |
| 181 | ggagataaaa caaataaaaa gaaagagaaa gagcggccag agatttctct cccttcagat |
| 241 | tttgaacaca caattcatgt cggttttgat gctgtcacag gggagtttac gggaatgcca |
| 301 | gagcagtggg cccgcttgct tcagacatca aatatcacta agtcggagca gaagaaaaac |
| 361 | ccgcaggctg ttctggatgt gttggagttt tacaactcga agaagacatc caacagccag |
| 421 | aaatacatga gctttacaga taagtcagct gaggattaca attcttctaa tgccttgaat |
| 481 | gtgaaggctg tgtctgagac tcctgcagtg ccaccagttt cagaagatga ggatgatgat |
| 541 | gatgatgatg ctaccccacc accagtgatt gctccacgcc cagagcacac aaaatctgta |
| 601 | tacacacggt ctgtgattga accacttcct gtcactccaa ctcgggacgt ggctacatct |
| 661 | cccatttcac ctactgaaaa taacaccact ccaccagatg ctttgacccg gaatactgag |
| 721 | aagcagaaga agaagcctaa aatgtctgat gaggagatct tggagaaatt acgaagcata |
| 781 | gtgagtgtgg gcgatcctaa gaagaaatat acacggtttg agaagattgg acaaggtgct |
| 841 | tcaggcaccg tgtacacagc aatggatgtg gccacaggac aggaggtggc cattaagcag |
| 901 | atgaatcttc agcagcagcc caagaaagag ctgattatta atgagatcct ggtcatgagg |
| 961 | gaaaacaaga acccaaacat tgtgaattac ttggacagtt acctcgtggg agatgagctg |
| 1021 | tgggttgtta tggaatactt ggctggaggc tccttgacag atgtggtgac agaaacttgc |
| 1081 | atggatgaag gccaaattgc agctgtgtgc cgtgagtgtc tgcaggctct ggagttcttg |
| 1141 | cattcgaacc aggtcattca cagagacatc aagagtgaca atattctgtt gggaatggat |
| 1201 | ggctctgtca agctaactga ctttggattc tgtgcacaga taaccccaga gcagagcaaa |
| 1261 | cggagcacca tggtaggaac cccatactgg atggcaccag aggttgtgac acgaaaggcc |
| 1321 | tatgggccca aggttgacat ctggtccctg ggcatcatgg ccatcgaaat gattgaaggg |
| 1381 | gagcctccat acctcaatga aaaccctctg agagccttgt acctcattgc caccaatggg |
| 1441 | accccagaac ttcagaaccc agagaagctg tcagctatct tccgggactt tctgaaccgc |
| 1501 | tgtctcgaga tggatgtgga gaagagaggt tcagctaaag agctgctaca gcatcaattc |
| 1561 | ctgaagattg ccaagcccct ctccagcctc actccactga ttgctgcagc taaggaggca |
| 1621 | acaaagaaca atcactaa |
| PAK1 isoform 2 amino acid sequence (GenBank Acc. Num. NP_002567.3; | |
| SEQ ID NO: 12): | |
| 1 | msnngldiqd kppappmrnt stmigagskd agtlnhgskp lppnpeekkk kdrfyrsilp |
| 61 | gdktnkkkek erpeislpsd fehtihvgfd avtgeftgmp eqwarllqts nitkseqkkn |
| 121 | pqavldvlef ynskktsnsq kymsftdksa edynssnaln vkavsetpav ppvsededdd |
| 181 | dddatpppvi aprpehtksv ytrsvieplp vtptrdvats pisptenntt ppdaltrnte |
| 241 | kqkkkpkmsd eeileklrsi vsvgdpkkky trfekigqga sgtvytamdv atgqevaikq |
| 301 | mnlqqqpkke liineilvmr enknpnivny ldsylvgdel wvvmeylagg sltdvvtetc |
| 361 | mdegqiaavc reclqalefl hsnqvihrdi ksdnillgmd gsvkltdfgf caqitpeqsk |
| 421 | rstmvgtpyw mapevvtrka ygpkvdiwsl gimaiemieg eppylnenpl ralyliatng |
| 481 | tpelqnpekl saifrdflnr clemdvekrg sakellqhqf lkiakplssl tpliaaakea |
| 541 | tknnh |
| ROCK1 cDNA nucleic acid sequence (GenBank Acc. Num. NM_005406.2; | |
| SEQ ID NO: 13): | |
| 1 | atgtcgactg gggacagttt tgagactcga tttgaaaaaa tggacaacct gctgcgggat |
| 61 | cccaaatcgg aagtgaattc ggattgtttg ctggatggat tggatgcttt ggtatatgat |
| 121 | ttggattttc ctgccttaag aaaaaacaaa aatattgaca actttttaag cagatataaa |
| 181 | gacacaataa ataaaatcag agatttacga atgaaagctg aagattatga agtagtgaag |
| 241 | gtgattggta gaggtgcatt tggagaagtt caattggtaa ggcataaatc caccaggaag |
| 301 | gtatatgcta tgaagcttct cagcaaattt gaaatgataa agagatctga ttctgctttt |
| 361 | ttctgggaag aaagggacat catggctttt gccaacagtc cttgggttgt tcagcttttt |
| 421 | tatgcattcc aagatgatcg ttatctctac atggtgatgg aatacatgcc tggtggagat |
| 481 | cttgtaaact taatgagcaa ctatgatgtg cctgaaaaat gggcacgatt ctatactgca |
| 541 | gaagtagttc ttgcattgga tgcaatccat tccatgggtt ttattcacag agatgtgaag |
| 601 | cctgataaca tgctgctgga taaatctgga catttgaagt tagcagattt tggtacttgt |
| 661 | atgaagatga ataaggaagg catggtacga tgtgatacag cggttggaac acctgattat |
| 721 | atttcccctg aagtattaaa atcccaaggt ggtgatggtt attatggaag agaatgtgac |
| 781 | tggtggtcgg ttggggtatt tttatacgaa atgcttgtag gtgatacacc tttttatgca |
| 841 | gattctttgg ttggaactta cagtaaaatt atgaaccata aaaattcact tacctttcct |
| 901 | gatgataatg acatatcaaa agaagcaaaa aaccttattt gtgccttcct tactgacagg |
| 961 | gaagtgaggt tagggcgaaa tggtgtagaa gaaatcaaac gacatctctt cttcaaaaat |
| 1021 | gaccagtggg cttgggaaac gctccgagac actgtagcac cagttgtacc cgatttaagt |
| 1081 | agtgacattg atactagtaa ttttgatgac ttggaagaag ataaaggaga ggaagaaaca |
| 1141 | ttccctattc ctaaagcttt cgttggcaat caactacctt ttgtaggatt tacatattat |
| 1201 | agcaatcgta gatacttatc ttcagcaaat cctaatgata acagaactag ctccaatgca |
| 1261 | gataaaagct tgcaggaaag tttgcaaaaa acaatctata agctggaaga acagctgcat |
| 1321 | aatgaaatgc agttaaaaga tgaaatggag cagaagtgca gaacctcaaa cataaaacta |
| 1381 | gacaagataa tgaaagaatt ggatgaagag ggaaatcaaa gaagaaatct agaatctaca |
| 1441 | gtgtctcaga ttgagaagga gaaaatgttg ctacagcata gaattaatga gtaccaaaga |
| 1501 | aaagctgaac aggaaaatga gaagagaaga aatgtagaaa atgaagtttc tacattaaag |
| 1561 | gatcagttgg aagacttaaa gaaagtcagt cagaattcac agcttgctaa tgagaagctg |
| 1621 | tcccagttac aaaagcagct agaagaagcc aatgacttac ttaggacaga atcggacaca |
| 1681 | gctgtaagat tgaggaagag tcacacagag atgagcaagt caattagtca gttagagtcc |
| 1741 | ctgaacagag agttgcaaga gagaaatcga attttagaga attctaagtc acaaacagac |
| 1801 | aaagattatt accagctgca agctatatta gaagctgaac gaagagacag aggtcatgat |
| 1861 | tctgagatga ttggagacct tcaagctcga attacatctt tacaagagga ggtgaagcat |
| 1921 | ctcaaacata atctcgaaaa agtggaagga gaaagaaaag aggctcaaga catgcttaat |
| 1981 | cactcagaaa aggaaaagaa taatttagag atagatttaa actacaaact taaatcatta |
| 2041 | caacaacggt tagaacaaga ggtaaatgaa cacaaagtaa ccaaagctcg tttaactgac |
| 2101 | aaacatcaat ctattgaaga ggcaaagtct gtggcaatgt gtgagatgga aaaaaagctg |
| 2161 | aaagaagaaa gagaagctcg agagaaggct gaaaatcggg ttgttcagat tgagaaacag |
| 2221 | tgttccatgc tagacgttga tctgaagcaa tctcagcaga aactagaaca tttgactgga |
| 2281 | aataaagaaa ggatggagga tgaagttaag aatctaaccc tgcaactgga gcaggaatca |
| 2341 | aataagcggc tgttgttaca aaatgaattg aagactcaag catttgaggc agacaattta |
| 2401 | aaaggtttag aaaagcagat gaaacaggaa ataaatactt tattggaagc aaagagatta |
| 2461 | ttagaatttg agttagctca gcttacgaaa cagtatagag gaaatgaagg acagatgcgg |
| 2521 | gagctacaag atcagcttga agctgagcaa tatttctcga cactttataa aacccaggta |
| 2581 | aaggaactta aagaagaaat tgaagaaaaa aacagagaaa atttaaagaa aatacaggaa |
| 2641 | ctacaaaatg aaaaagaaac tcttgctact cagttggatc tagcagaaac aaaagctgag |
| 2701 | tctgagcagt tggcgcgagg ccttctggaa gaacagtatt ttgaattgac gcaagaaagc |
| 2761 | aagaaagctg cttcaagaaa tagacaagag attacagata aagatcacac tgttagtcgg |
| 2821 | cttgaagaag caaacagcat gctaaccaaa gatattgaaa tattaagaag agagaatgaa |
| 2881 | gagctaacag agaaaatgaa gaaggcagag gaagaatata aactggagaa ggaggaggag |
| 2941 | atcagtaatc ttaaggctgc ctttgaaaag aatatcaaca ctgaacgaac ccttaaaaca |
| 3001 | caggctgtta acaaattggc agaaataatg aatcgaaaag attttaaaat tgatagaaag |
| 3061 | aaagctaata cacaagattt gagaaagaaa gaaaaggaaa atcgaaagct gcaactggaa |
| 3121 | ctcaaccaag aaagagagaa attcaaccag atggtagtga aacatcagaa ggaactgaat |
| 3181 | gacatgcaag cgcaattggt agaagaatgt gcacatagga atgagcttca gatgcagttg |
| 3241 | gccagcaaag agagtgatat tgagcaattg cgtgctaaac ttttggacct ctcggattct |
| 3301 | acaagtgttg ctagttttcc tagtgctgat gaaactgatg gtaacctccc agagtcaaga |
| 3361 | attgaaggtt ggctttcagt accaaataga ggaaatatca aacgatatgg ctggaagaaa |
| 3421 | cagtatgttg tggtaagcag caaaaaaatt ttgttctata atgacgaaca agataaggag |
| 3481 | caatccaatc catctatggt attggacata gataaactgt ttcacgttag acctgtaacc |
| 3541 | caaggagatg tgtatagagc tgaaactgaa gaaattccta aaatattcca gatactatat |
| 3601 | gcaaatgaag gtgaatgtag aaaagatgta gagatggaac cagtacaaca agctgaaaaa |
| 3661 | actaatttcc aaaatcacaa aggccatgag tttattccta cactctacca ctttcctgcc |
| 3721 | aattgtgatg cctgtgccaa acctctctgg catgttttta agccaccccc tgccctagag |
| 3781 | tgtcgaagat gccatgttaa gtgccacaga gatcacttag ataagaaaga ggacttaatt |
| 3841 | tgtccatgta aagtaagtta tgatgtaaca tcagcaagag atatgctgct gttagcatgt |
| 3901 | tctcaggatg aacaaaaaaa atgggtaact catttagtaa agaaaatccc taagaatcca |
| 3961 | ccatctggtt ttgttcgtgc ttcccctcga acgctttcta caagatccac tgcaaatcag |
| 4021 | tctttccgga aagtggtcaa aaatacatct ggaaaaacta gttaa |
| ROCK1 amino acid sequence (GenBank Acc. Num. NP_005397.1; SEQ ID NO: 14): | |
| 1 | mstgdsfetr fekmdnllrd pksevnsdcl ldgldalvyd ldfpalrknk nidnflsryk |
| 61 | dtinkirdlr mkaedyevvk vigrgafgev qlvrhkstrk vyamkllskf emikrsdsaf |
| 121 | fweerdimaf anspwvvqlf yafqddryly mvmeympggd lvnlmsnydv pekwarfyta |
| 181 | evvlaldaih smgfihrdvk pdnmlldksg hlkladfgtc mkmnkegmvr cdtavgtpdy |
| 241 | ispevlksqg gdgyygrecd wwsvgvflye mLvgdtpfya dslvgtyski mnhknsltfp |
| 301 | ddndiskeak nlicafltdr evrlgrngve eikrhlffkn dqwawetlrd tvapvvpdls |
| 361 | sdidtsnfdd leedkgeeet fpipkafvgn qlpfvgftyy snrrylssan pndnrtssna |
| 421 | dkslqeslqk tiykleeqlh nemqlkdeme qkcrtsnikl dkimkeldee gnqrrnlest |
| 481 | vsqiekekml lqhrineyqr kaeqenekrr nvenevstlk dqledlkkvs qnsqlanekl |
| 541 | sqlqkqleea ndllrtesdt avrlrkshte msksisqles lnrelqernr ilensksqtd |
| 601 | kdyyqlqail eaerrdrghd semigdlqar itslqeevkh lkhnlekveg erkeaqdmln |
| 661 | hsekeknnle idlnyklksl qqrleqevne hkvtkarltd khqsieeaks vamcemekkl |
| 721 | keereareka enrvvqiekq csmldvdlkq sqqklehltg nkermedevk nltlqleqes |
| 781 | nkrlllqnel ktqafeadnl kglekqmkqe intlleakrl lefelaqltk qyrgnegqmr |
| 841 | elqdqleaeq yfstlyktqv kelkeeieek nrenlkkiqe lqneketlat qldlaetkae |
| 901 | seqlarglle eqyfeltqes kkaasrnrqe itdkdhtvsr leeansmltk dieilrrene |
| 961 | eltekmkkae eeyklekeee isnlkaafek nintertlkt qavnklaeim nrkdfkidrk |
| 1021 | kantqdlrkk ekenrklqle lnqerekfnq mvvkhqkeln dmqaqlveec ahrnelqmql |
| 1081 | askesdieql raklldlsds tsvasfpsad etdgnlpesr iegwlsvpnr gnikrygwkk |
| 1141 | qyvvvsskki lfyndeqdke qsnpsmvldi dklfhvrpvt qgdvyraete eipkifqily |
| 1201 | anegecrkdv emepvqqaek tnfqnhkghe fiptlyhfpa ncdacakplw hvfkpppale |
| 1261 | crrchvkchr dhldkkedli cpckvsydvt sardmlllac sqdeqkkwvt hlvkkipknp |
| 1321 | psgfvraspr tlstrstanq sfrkvvknts gkts |
| ROCK2 cDNA nucleic acid sequence (GenBank Acc. Num. NM_004850.3; | |
| SEQ ID NO: 15): | |
| 1 | atgagccggc ccccgccgac ggggaaaatg cccggcgccc ccgagaccgc gccgggggac |
| 61 | ggggcaggcg cgagccgcca gaggaagctg gaggcgctga tccgagaccc tcgctccccc |
| 121 | atcaacgtgg agagcttgct ggatggctta aattccttgg tccttgattt agattttcct |
| 181 | gctttgagga aaaacaagaa catagataat ttcttaaata gatatgagaa aattgtgaaa |
| 241 | aaaatcagag gtctacagat gaaggcagaa gactatgatg ttgtaaaagt tattggaaga |
| 301 | ggtgcttttg gtgaagtgca gttggttcgt cacaaggcat cgcagaaggt ttatgctatg |
| 361 | aagcttctta gtaagtttga aatgataaaa agatcagatt ctgccttttt ttgggaagaa |
| 421 | agagatatta tggcctttgc caatagcccc tgggtggttc agctttttta tgcctttcaa |
| 481 | gatgataggt atctgtacat ggtaatggag tacatgcctg gtggagacct tgtaaacctt |
| 541 | atgagtaatt atgatgtgcc tgaaaaatgg gccaaatttt acactgctga agttgttctt |
| 601 | gctctggatg caatacactc catgggttta atacacagag atgtgaagcc tgacaacatg |
| 661 | ctcttggata aacatggaca tctaaaatta gcagattttg gcacgtgtat gaagatggat |
| 721 | gaaacaggca tggtacattg tgatacagca gttggaacac cggattatat atcacctgag |
| 781 | gttctgaaat cacaaggggg tgatggtttc tatgggcgag aatgtgattg gtggtctgta |
| 841 | ggtgttttcc tttatgagat gctagtgggg gatactccat tttatgcgga ttcacttgta |
| 901 | ggaacatata gcaaaattat ggatcataag aattcactgt gtttccctga agatgcagaa |
| 961 | atttccaaac atgcaaagaa tctcatctgt gctttcttaa cagataggga ggtacgactt |
| 1021 | gggagaaatg gggtggaaga aatcagacag catcctttct ttaagaatga tcagtggcat |
| 1081 | tgggataaca taagagaaac ggcagctcct gtagtacctg aactcagcag tgacatagac |
| 1141 | agcagcaatt tcgatgacat tgaagatgac aaaggagatg tagaaacctt cccaattcct |
| 1201 | aaagcttttg ttggaaatca gctgcctttc atcggattta cctactatag agaaaattta |
| 1261 | ttattaagtg actctccatc ttgtagagaa actgattcca tacaatcaag gaaaaatgaa |
| 1321 | gaaagtcaag agattcagaa aaaactgtat acattagaag aacatcttag caatgagatg |
| 1381 | caagccaaag aggaactgga acagaagtgc aaatctgtta atactcgcct agaaaaaaca |
| 1441 | gcaaaggagc tagaagagga gattacctta cggaaaagtg tggaatcagc attaagacag |
| 1501 | ttagaaagag aaaaggcgct tcttcagcac aaaaatgcag aatatcagag gaaagctgat |
| 1561 | catgaagcag acaaaaaacg aaatttggaa aatgatgtta acagcttaaa agatcaactt |
| 1621 | gaagatttga aaaaaagaaa tcaaaactct caaatatcca ctgagaaagt gaatcaactc |
| 1681 | cagagacaac tggatgaaac caatgcttta ctgcgaacag agtctgatac tgcagcccgg |
| 1741 | ttaaggaaaa cccaggcaga aagttcaaaa cagattcagc agctggaatc taacaataga |
| 1801 | gatctacaag ataaaaactg cctgctggag actgccaagt taaaacttga aaaggaattt |
| 1861 | atcaatcttc agtcagctct agaatctgaa aggagggatc gaacccatgg atcagagata |
| 1921 | attaatgatt tacaaggtag aatatgtggc ctagaagaag atttaaagaa cggcaaaatc |
| 1981 | ttactagcga aagtagaact ggagaagaga caacttcagg agagatttac tgatttggaa |
| 2041 | aaggaaaaaa gcaacatgga aatagatatg acataccaac taaaagttat acagcagagc |
| 2101 | ctagaacaag aagaagctga acataaggcc acaaaggcac gactagcaga taaaaataag |
| 2161 | atctatgagt ccatcgaaga agccaaatca gaagccatga aagaaatgga gaagaagctc |
| 2221 | ttggaggaaa gaactttaaa acagaaagtg gagaacctat tgctagaagc tgagaaaaga |
| 2281 | tgttctctat tagactgtga cctcaaacag tcacagcaga aaataaatga gctccttaaa |
| 2341 | cagaaagatg tgctaaatga ggatgttaga aacctgacat taaaaataga gcaagaaact |
| 2401 | cagaagcgct gccttacaca aaatgacctg aagatgcaaa cacaacaggt taacacacta |
| 2461 | aaaatgtcag aaaagcagtt aaagcaagaa aataaccate tcatggaaat gaaaatgaac |
| 2521 | ttggaaaaac aaaatgctga acttcgaaaa gaacgtcagg atgcagatgg gcaaatgaaa |
| 2581 | gagctccagg atcagctcga agcagaacag tatttctcaa ccctttataa aacacaagtt |
| 2641 | agggagctta aagaagaatg tgaagaaaag accaaacttg gtaaagaatt gcagcagaag |
| 2701 | aaacaggaat tacaggatga acgggactct ttggctgccc aactggagat caccttgacc |
| 2761 | aaagcagatt ctgagcaact ggctcgttca attgctgaag aacaatattc tgatttggaa |
| 2821 | aaagagaaga tcatgaaaga gctggagatc aaagagatga tggctagaca caaacaggaa |
| 2881 | cttacggaaa aagatgctac aattgcttct cttgaggaaa ctaataggac actaactagt |
| 2941 | gatgttgcca atcttgcaaa tgagaaagaa gaattaaata acaaattgaa agatgttcaa |
| 3001 | gagcaactgt caagattgaa agatgaagaa ataagcgcag cagctattaa agcacagttt |
| 3061 | gagaagcagc tattaacaga aagaacactc aaaactcaag ctgtgaataa gttggctgag |
| 3121 | atcatgaatc gaaaagaacc tgtcaagcgt ggtaatgaca cagatgtgcg gagaaaagag |
| 3181 | aaggagaata gaaagctaca tatggagctt aaatctgaac gtgagaaatt gacccagcag |
| 3241 | atgatcaagt atcagaaaga actgaatgaa atgcaggcac aaatagctga agagagccag |
| 3301 | attcgaattg aactgcagat gacattggac agtaaagaca gtgacattga gcagctgcgg |
| 3361 | tcacaactcc aagccttgca tattggtctg gatagttcca gtataggcag tggaccaggg |
| 3421 | gatgctgagg cagatgatgg gtttccagaa tcaagattag aaggatggct ttcattgcct |
| 3481 | gtacgaaaca acactaagaa atttggatgg gttaaaaagt atgtgattgt aagcagtaag |
| 3541 | aagattcttt tctatgacag tgaacaagat aaagaacaat ccaatcctta catggtttta |
| 3601 | gatatagaca agttatttca tgtccgacca gttacacaga cagatgtgta tagagcagat |
| 3661 | gctaaagaaa ttccaaggat attccagatt ctgtatgcca atgaaggaga aagtaagaag |
| 3721 | gaacaagaat ttccagtgga gccagttgga gaaaaatcta attatatttg ccacaaggga |
| 3781 | catgagttta ttcctactct ttatcatttc ccaaccaact gtgaggcttg tatgaagccc |
| 3841 | ctgtggcaca tgtttaagcc tcctcctgct ttggagtgcc gccgttgcca tattaagtgt |
| 3901 | cataaagatc atatggacaa aaaggaggag attatagcac cttgcaaagt atattatgat |
| 3961 | atttcaacgg caaagaatct gttattacta gcaaattcta cagaagagca gcagaagtgg |
| 4021 | gttagtcggt tggtgaaaaa gatacctaaa aagcccccag ctccagaccc ttttgcccga |
| 4081 | tcatctccta gaacttcaat gaagatacag caaaaccagt ctattagacg gccaagtcga |
| 4141 | cagcttgccc caaacaaacc tagctaa |
| ROCK2 amino acid sequence (GenBank Acc. Num. NP_004841.2; SEQ ID NO: 16): | |
| 1 | msrppptgkm pgapetapgd gagasrqrkl ealirdprsp inveslldgl nslvldldfp |
| 61 | alrknknidn flnryekivk kirglqmkae dydvvkvigr gafgevqlvr hkasqkvyam |
| 121 | kllskfemik rsdsaffwee rdimafansp wvvqlfyafq ddrylymvme ympggdlvnl |
| 181 | msnydvpekw akfytaevvl aldaihsmgl ihrdvkpdnm lldkhghlkl adfgtcmkmd |
| 241 | etgmvhcdta vgtpdyispe vlksqggdgf ygrecdwwsv gvflyemlvg dtpfyadslv |
| 301 | gtyskimdhk nslcfpedae iskhaknlic afltdrevrl grngveeirq hpffkndqwh |
| 361 | wdniretaap vvpelssdid ssnfddiedd kgdvetfpip kafvgnqlpf igftyyrenl |
| 421 | llsdspscre tdsiqsrkne esqeiqkkly tleehlsnem qakeeleqkc ksvntrlekt |
| 481 | akeleeeitl rksvesalrq lerekallqh knaeyqrkad headkkrnle ndvnslkdql |
| 541 | edlkkrnqns qistekvnql qrqldetnal lrtesdtaar lrktqaessk qiqqlesnnr |
| 601 | dlqdknclle taklklekef inlqsalese rrdrthgsei indlqgricg leedlkngki |
| 661 | llakvelekr qlqerftdle keksnmeidm tyqlkviqqs leqeeaehka tkarladknk |
| 721 | iyesieeaks eamkemekkl leertlkqkv enllleaekr cslldcdlkq sqqkinellk |
| 781 | qkdvlnedvr nltlkieqet qkrcltqndl kmqtqqvntl kmsekqlkqe nnhlmemkmn |
| 841 | lekqnaelrk erqdadgqmk elqdqleaeq yfstlyktqv relkeeceek tklgkelqqk |
| 901 | kqelqderds laaqleitlt kadseqlars iaeeqysdle kekimkelei kemmarhkqe |
| 961 | ltekdatias leetnrtlts dvanlaneke elnnklkdvq eqlsrlkdee isaaaikaqf |
| 1021 | ekqlltertl ktqavnklae imnrkepvkr gndtdvrrke kenrklhmel kserekltqq |
| 1081 | mikyqkelne mqaqiaeesq irielqmtld skdsdieqlr sqlqalhigl dsssigsgpg |
| 1141 | daeaddgfpe srlegwlslp vrnntkkfgw vkkyvivssk kilfydseqd keqsnpymvl |
| 1201 | didklfhvrp vtqtdvyrad akeiprifqi lyanegeskk eqefpvepvg eksnyichkg |
| 1261 | hefiptlyhf ptnceacmkp lwhmfkpppa lecrrchikc hkdhmdkkee iiapckvyyd |
| 1321 | istaknllll ansteeqqkw vsrlvkkipk kppapdpfar ssprtsmkiq qnqsirrpsr |
| 1381 | qlapnkps |

II. Compositions and Agents

Compositions and agents of the present invention are provided for us in the diagnosis, prognosis, prevention, and treatment of cancer and cancer subtypes thereof. Such compositions and agents can detect and/or modulate, e.g., up- or down-regulate, expression and/or activity of gene products or fragments thereof encoded by biomarkers of the present invention listed or described herein. Exemplary agents include antibodies, small molecules, peptides, peptidomimetics, natural ligands, and derivatives of natural ligands, that can either bind and/or activate or inhibit protein biomarkers of the invention, including the biomarkers listed or described herein, or fragments thereof, and nucleic acid molecules, such as RNA interference molecules, antisense molecules, nucleic acid aptamers, etc. that can downregulate the expression and/or activity of the biomarkers of the invention, including the biomarkers listed or described herein, or fragments thereof.

In one embodiment, isolated nucleic acid molecules are provided that specifically hybridize with or encode one or more biomarkers listed or described herein or biologically active portions thereof. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules corresponding to the one or more biomarkers listed or described herein can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., a leukemic cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having the nucleotide sequence of one or more biomarkers listed or described herein or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence of one or more biomarkers listed or described herein or a portion thereof (i.e., 100, 200, 300, 400, 450, 500, or more nucleotides), can be isolated using standard molecular biology techniques and the sequence information provided herein. In some embodiments, the nucleic acid sequences encodes the wild-type biomarker protein, except for 1, 2, 3, 4, 5, 6, 7, 8, 9, 10, 11, 12, 13, 14, 15, or fewer amino acid changes. For example, a human cDNA can be isolated from a human cell line (from Stratagene, La Jolla, Calif., or Clontech, Palo Alto, Calif.) using all or portion of the nucleic acid molecule, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989). Moreover, a nucleic acid molecule encompassing all or a portion of the nucleotide sequence of one or more biomarkers listed or described herein or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence, or fragment thereof, can be isolated by the polymerase chain reaction using oligonucleotide primers designed based upon the sequence of the one or more biomarkers listed or described herein, or fragment thereof, or the homologous nucleotide sequence. For example, mRNA can be isolated from muscle cells (i.e., by the guanidinium-thiocyanate extraction procedure of Chirgwin et al. (1979) Biochemistry 18: 5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed according to well-known methods in the art. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequence of one or more biomarkers listed or described herein can be prepared by standard synthetic techniques, i.e., using an automated DNA synthesizer.

Probes based on the nucleotide sequences of one or more biomarkers listed or described herein can be used to detect transcripts or genomic sequences encoding the same or homologous proteins. In preferred embodiments, the probe further comprises a label group attached thereto, i.e., the label group can be a radioisotope, a fluorescent compound, an enzyme, or an enzyme co-factor. Such probes can be used as a part of a diagnostic test kit for identifying cells or tissue which express one or more biomarkers listed or described herein, such as by measuring a level of nucleic acid in a sample of cells from a subject, i.e., detecting mRNA levels of one or more biomarkers listed or described herein.

Nucleic acid molecules encoding proteins corresponding to one or more biomarkers listed or described herein from different species are also contemplated. For example, rat or monkey cDNA can be identified based on the nucleotide sequence of a human and/or mouse sequence and such sequences are well known in the art. In one embodiment, the nucleic acid molecule(s) of the invention encodes a protein or portion thereof which includes an amino acid sequence which is sufficiently homologous to an amino acid sequence of one or more biomarkers listed or described herein, such that the protein or portion thereof modulates (e.g., enhance), one or more of the following biological activities: a) binding to the biomarker; b) modulating the copy number of the biomarker; c) modulating the expression level of the biomarker; and d) modulating the activity level of the biomarker.

In some embodiments, the biomarkers of the present invention (e.g., mutant RAC proteins or nucleic acid molecules encoding same) described herein are further defined as having the ability to be constitutively active and oncogenic. A “constitutively active” polypeptide is one exhibiting constant expression of the activity (e.g., unregulated expression of an activity normally regulated in the corresponding wild-type polypeptide). An “oncogenic” polypeptide is one having the ability to render a cell hyperproliferative. Activities of RAC polypeptides are well-known in the art and include, without limitation, the ability to 1) hydrolyze guanosine triphosphate (GTP), 2) regulate cell growth, 3) regulate the cell cycle, 4) regulate epithelial differentiation, 5) reorganize the cellular cytoskeleton, and 6) activating protein kinases. As compared to the corresponding wild-type polypeptide, mutant RAC markers described herein can have altered amount, structure, subcellular localization, and/or activity of at least 50%, 60%, 70%, 80%, 90%, 100%, 150%, 200%, 300%, 400%, 500%, 1000% or more in excess of that relative to the corresponding wild-type RAC marker. Exemplary methods for determining activity levels and oncogenicity are well-known in the art and described further herein (e.g., anchorage-independent growth assays, soft agar assays, nude mouse tumorigenicity assays, immunoblot assays, RNA knockdown assays, GTP-loading assays, cytoskeletal reorganization assays, and the like).

As used herein, the language “sufficiently homologous” refers to proteins or portions thereof which have amino acid sequences which include a minimum number of identical or equivalent (e.g., an amino acid residue which has a similar side chain as an amino acid residue in one or more biomarkers listed or described herein, or fragment thereof) amino acid residues to an amino acid sequence of the biomarker, or fragment thereof, such that the protein or portion thereof modulates (e.g., enhance) one or more of the following biological activities: a) binding to the biomarker; b) modulating the copy number of the biomarker; c) modulating the expression level of the biomarker; d) modulating the activity level of the biomarker; and/or e) modulating the constitutively active and/or oncogenic status of the biomarker.

In another embodiment, the protein is at least about 50%, preferably at least about 60%, more preferably at least about 60%, 70%, 75%, 80%, 85%, 90%, 91%, 92%, 93%, 94%, 95%, 96%, 97%, 98%, 99% or more homologous to the entire amino acid sequence of the biomarker, or a fragment thereof. In other embodiments, the protein differs from the amino acid sequence of the wild-type protein, except for 1, 2, 3, 4, 5, 6, 7, 8, 9, 10, 11, 12, 13, 14, 15, or fewer amino acids. For example, a mutant RAC polypeptide can have any combination of amino acid substitutions, deletions or insertions. Numerous such mutant RAC polypeptides are described herein using amino acid position nomenclature for the human RAC proteins, although corresponding amino acid residues in orthologs of non-human species can easily be identified and generated and are withing the scope of the present invention. In one embodiment, isolated mutant RAC polypeptides have an integer number of amino acid alterations such that their amino acid sequence shares at least 60, 70, 80, 85, 90, 95, 96, 97, 98, 99, 99.5 or 100% identity with an amino acid sequence of a wild-type RAC polypeptide. As described herein, RAC markers are highly conserved among species and the structures are known such that the skilled artisan would readily understand which regions of the RAC markers can be altered without affecting a desired function mediated by RAC.

Portions of proteins encoded by nucleic acid molecules of the one or more biomarkers listed or described herein are preferably biologically active portions of the protein. As used herein, the term “biologically active portion” of one or more biomarkers listed or described herein is intended to include a portion, e.g., a domain/motif, that has one or more of the biological activities of the full-length protein. For example, mutant RAC markers described herein can correspond to a full-length RAC marker, or can be a fragment of a full-length RAC marker. A “fragment” of a RAC marker refers to any subset of the marker that is shorter than the full-length RAC marker. In one embodiment, mutant RAC polypeptides are those that retain the ability to be constitutively active and oncogenic. Such RAC marker fragments can be, for example, 5, 10, 15, 20, 25, 30, 35, 40, 45, 50, or more amino acids shorter than the corresponding full-length RAC marker.

Standard binding assays, e.g., immunoprecipitations and yeast two-hybrid assays, as described herein, or functional assays, e.g., RNAi or overexpression experiments, can be performed to determine the ability of the protein or a biologically active fragment thereof to maintain a biological activity of the full-length protein.

The invention further encompasses nucleic acid molecules that differ from the nucleotide sequence of the one or more biomarkers listed or described herein, or fragment thereof due to degeneracy of the genetic code and thus encode the same protein as that encoded by the nucleotide sequence, or fragment thereof. In another embodiment, an isolated nucleic acid molecule of the invention has a nucleotide sequence encoding a protein having an amino acid sequence of one or more biomarkers listed or described herein, or fragment thereof, or a protein having an amino acid sequence which is at least about 70%, 75%, 80%, 85%, 90%, 91%, 92%, 93%, 94%, 95%, 96%, 97%, 98%, 99% or more homologous to the amino acid sequence of the one or more biomarkers listed or described herein, or fragment thereof. In another embodiment, a nucleic acid encoding a polypeptide consists of nucleic acid sequence encoding a portion of a full-length fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of the one or more biomarkers listed or described herein may exist within a population (e.g., a mammalian and/or human population). Such genetic polymorphisms may exist among individuals within a population due to natural allelic variation. As used herein, the terms “gene” and “recombinant gene” refer to nucleic acid molecules comprising an open reading frame encoding one or more biomarkers listed or described herein, preferably a mammalian, e.g., human, protein. Such natural allelic variations can typically result in 1-5% variance in the nucleotide sequence of the one or more biomarkers listed or described herein. Any and all such nucleotide variations and resulting amino acid polymorphisms in the one or more biomarkers listed or described herein that are the result of natural allelic variation and that do not alter the functional activity of the one or more biomarkers listed or described herein are intended to be within the scope of the invention. Moreover, nucleic acid molecules encoding one or more biomarkers listed or described herein from other species.

In addition to naturally-occurring allelic variants of the one or more biomarkers listed or described herein sequence that may exist in the population, the skilled artisan will further appreciate that changes can be introduced by mutation into the nucleotide sequence, or fragment thereof, thereby leading to changes in the amino acid sequence of the encoded one or more biomarkers listed or described herein, without altering the functional ability of the one or more biomarkers listed or described herein. For example, nucleotide substitutions leading to amino acid substitutions at “non-essential” amino acid residues can be made in the sequence, or fragment thereof. A “non-essential” amino acid residue is a residue that can be altered from the wild-type sequence of the one or more biomarkers listed or described herein without altering the activity of the one or more biomarkers listed or described herein, whereas an “essential” amino acid residue is required for the activity of the one or more biomarkers listed or described herein. Other amino acid residues, however, (e.g., those that are not conserved or only semi-conserved between mouse and human) may not be essential for activity and thus are likely to be amenable to alteration without altering the activity of the one or more biomarkers listed or described herein. Amino acid substitutions in polypeptides of the present invention may also be “conservative” or “non-conservative.” “Conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glut, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a protein homologous to one or more biomarkers listed or described herein, or fragment thereof, can be created by introducing one or more nucleotide substitutions, additions or deletions into the nucleotide sequence, or fragment thereof, or a homologous nucleotide sequence such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. Thus, a predicted nonessential amino acid residue in one or more biomarkers listed or described herein is preferably replaced with another amino acid residue from the same side chain family. Alternatively, in another embodiment, mutations can be introduced randomly along all or part of the coding sequence of the one or more biomarkers listed or described herein, such as by saturation mutagenesis, and the resultant mutants can be screened for an activity described herein to identify mutants that retain desired activity. Following mutagenesis, the encoded protein can be expressed recombinantly according to well-known methods in the art and the activity of the protein can be determined using, for example, assays described herein.

The levels of one or more biomarkers listed or described herein levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In preferred embodiments, the levels of one or more biomarkers listed or described herein levels are ascertained by measuring gene transcript (e.g., mRNA), by a measure of the quantity of translated protein, or by a measure of gene product activity. Expression levels can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In a particular embodiment, the mRNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding one or more biomarkers listed or described herein. Other suitable probes for use in the diagnostic assays of the invention are described herein. Hybridization of an mRNA with the probe indicates that one or more biomarkers listed or described herein is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of the One or more biomarkers listed or described herein mRNA expression levels.

An alternative method for determining mRNA expression level in a sample involves the process of nucleic acid amplification, e.g., by RT-PCR (the experimental embodiment set forth in Mullis, 1987, U.S. Pat. No. 4,683,202; incorporated by reference), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the One or more biomarkers listed or described herein mRNA.

As an alternative to making determinations based on the absolute expression level, determinations may be based on the normalized expression level of one or more biomarkers listed or described herein. Expression levels are normalized by correcting the absolute expression level by comparing its expression to the expression of a non-biomarker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a protein corresponding to one or more biomarkers listed or described herein can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express the biomarker of interest.

The present invention further provides soluble, purified and/or isolated polypeptide forms of one or more biomarkers listed or described herein, or fragments thereof. In addition, it is to be understood that any and all attributes of the polypeptides described herein, such as percentage identities, polypeptide lengths, polypeptide fragments, biological activities, antibodies, etc. can be combined in any order or combination with respect to any biomarker listed or described herein and combinations thereof.

In one aspect, a polypeptide may comprise a full-length amino acid sequence corresponding to one or more biomarkers listed or described herein or a full-length amino acid sequence with 1 to about 20 conservative amino acid substitutions. An amino acid sequence of any described herein can also be at least 50, 55, 60, 65, 70, 75, 80, 85, 90, 91, 92, 93, 94, 95, 96, 97, 98, 99, or 99.5% identical to the full-length sequence of one or more biomarkers listed or described herein, which is either described herein, well known in the art, or a fragment thereof. In another aspect, the present invention contemplates a composition comprising an isolated polypeptide corresponding to one or more biomarkers listed or described herein polypeptide and less than about 25%, or alternatively 15%, or alternatively 5%, contaminating biological macromolecules or polypeptides.

In addition, the biomarkers described herein can also be modified according to a number of well-known methods. For example, mutant RAC markers can be modified modified by chemical moieties that may be present in polypeptides in a normal cellular environment, for example, phosphorylation, methylation, amidation, sulfation, acylation, glycosylation, sumoylation and ubiquitylation. In some embodiments, the polypeptides can be also be modified with a label capable of providing a detectable signal, either directly or indirectly, including, but not limited to, radioisotopes and fluorescent compounds. Such polypeptides can also be modified by chemical moieties that are not normally added to polypeptides in a cellular environment. Such modifications can be introduced into the molecule by reacting targeted amino acid residues of the polypeptide with an organic derivatizing agent that is capable of reacting with selected side chains or terminal residues. Another modification is cyclization of the protein.

The present invention further provides compositions related to producing, detecting, characterizing, or modulating the level or activity of such polypeptides, or fragment thereof, such as nucleic acids, vectors, host cells, and the like. Such compositions may serve as compounds that modulate the expression and/or activity of one or more biomarkers listed or described herein.

An isolated polypeptide or a fragment thereof (or a nucleic acid encoding such a polypeptide) corresponding to one or more biomarkers of the invention, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

For example, a polypeptide immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBVhybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating a monoclonal antibody against one or more biomarkers of the invention, including the biomarkers listed or described herein, or a fragment thereof (see, e.g., Galfre, G. et al. (1977) Nature 266:550-52; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; all of which patents and published patent applications are incorporated by reference; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269 (incorporated by reference); Akira et al. European Patent Application 184,187; Taniguchi, M. European Patent Application 171,496; Morrison et al. European Patent Application 173,494; Neuberger et al. PCT Application WO 86/01533 (incorporated by reference); Cabilly et al. U.S. Pat. No. 4,816,567 (incorporated by reference); Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332 (incorporated by reference). In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743; all of which are incorporated by reference. The use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 (incorporated by reference) by Marasco et al.; and PCT Publication No. WO 95/03832 (incorporated by reference) by Duan et al.).

Additionally, fully human antibodies could be made against biomarkers of the invention, including the biomarkers listed or described herein, or fragments thereof. Fully human antibodies can be made in mice that are transgenic for human immunoglobulin genes, e.g., according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893 (incorporated by reference). Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084 (incorporated by reference). Fragments of bispecific antibodies are described in U.S. Pat. No. 5,798,229 (incorporated by reference).

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers of the invention, including one or more biomarkers listed or described herein, or a fragment thereof. In one embodiment, the bispecific antibody could specifically bind to both a polypeptide or a fragment thereof and its natural binding partner(s) or a fragment(s) thereof.

In another aspect of this invention, peptides or peptide mimetics can be used to antagonize or promote the activity of one or more biomarkers of the invention, including one or more biomarkers listed or described herein, or a fragment(s) thereof. In one embodiment, variants of one or more biomarkers listed or described herein which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers of the invention, including one or more biomarkers listed or described herein, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere, J. (1986) Adv. Drug Res. 15:29; Veber and Freidinger (1985) TINS p.392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2—, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appin. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivitization (e.g., labeling) of peptidomimetics should not substantially interfere with the desired biological or pharmacological activity of the peptidomimetic.

The invention also relates to chimeric or fusion proteins of the biomarkers of the invention, including the biomarkers listed or described herein, or fragments thereof. As used herein, a “chimeric protein” or “fusion protein” comprises one or more biomarkers of the invention, including one or more biomarkers listed or described herein, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers of the invention, including one or more biomarkers listed or described herein, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another embodiment, the first peptide consists of a portion of a biologically active molecule (e.g., the extracellular portion of the polypeptide or the ligand binding portion).

Preferably, a fusion protein of the invention is produced by standard recombinant DNA techniques.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can enhance or upregulate one or more biological activities associated with the corresponding wild-type, naturally occurring, or synthetic small nucleic acids. In another embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers of the invention, including one or more biomarkers listed or described herein, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, small nucleic acids and/or antisense oligonucleotides may comprise or be generated from double stranded small interfering RNAs (siRNAs), in which sequences fully complementary to cellular nucleic acids (e.g., mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO 90/11364 (incorporated by reference), published Oct. 4, 1990; Sarver et al. (1990) Science 247:1222-1225 and U.S. Pat. No. 5,093,246 (incorporated by reference)). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO 88/04300 (incorporated by reference) by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidinerich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. Based on the genetic pathway analyses described herein, it is believed that such combinations of agents is especially effective in diagnosing, prognosing, preventing, and treating cancer. Thus, “single active agents” described herein can be combined with other pharmacologically active compounds (“second active agents”) known in the art according to the methods and compositions provided herein. It is believed that certain combinations work synergistically in the treatment of particular types of cancer. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alpha-n1, interferon alpha-n3, interferon beta-Ia, and interferon gamma-Ib; GM-CF and GM-CSF; and EPO.

Particular proteins that can be used in the methods and compositions provided herein include, but are not limited to: filgrastim, which is sold in the United States under the trade name Neupogen (registered trademark) (Amgen, Thousand Oaks, Calif.); sargramostim, which is sold in the United States under the trade name Leukine (registered trademark) (Immunex, Seattle, Wash.); and recombinant EPO, which is sold in the United States under the trade name Epogen (registered trademark) (Amgen, Thousand Oaks, Calif.). Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

Antibodies that can be used in combination form include monoclonal and polyclonal antibodies. Examples of antibodies include, but are not limited to, trastuzumab (Herceptin (registered trademark)), rituximab (Rituxan (registered trademark)), bevacizumab (Avastin (registered trademark)), pertuzumab (Omnitarg (registered trademark)), tositumomab (Bexxar (registered trademark)), edrecolomab (Panorex (registered trademark)), and G250. Compounds of the invention can also be combined with, or used in combination with, anti-TNF-alpha antibodies. Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits provided herein. See, e.g., Emens, L. A., et al., Curr. Opinion Mol. Ther. 3(1):77-84 (2001).

Second active agents that are small molecules can also be used in combination as provided herein. Examples of small molecule second active agents include, but are not limited to, anti-cancer agents, antibiotics, immunosuppressive agents, and steroids.

In some embodiments, well known “combination chemotherapy” regimens can be used. In one embodiment, the combination chemotherapy comprises a combination of two or more of cyclophosphamide, hydroxydaunorubicin (also known as doxorubicin or adriamycin), oncovorin (vincristine), and prednisone. In another embodiment, the combination chemotherapy comprises a combination of cyclophsophamide, oncovorin, prednisone, and one or more chemotherapeutics selected from the group consisting of anthracycline, hydroxydaunorubicin, epirubicin, and motixantrone.

III. Methods of Selecting Agents and Compositions

Another aspect of the invention relates to methods of selecting agents (e.g., antibodies, fusion proteins, peptides, small molecules, or small nucleic acids) which bind to, upregulate, downregulate, or modulate one or more biomarkers of the invention listed or described herein and/or a cancer (e.g., a lymphoid cancer, such as leukemia). Such methods utilize can use screening assays, including cell based and non-cell based assays.

In one embodiment, the invention relates to assays for screening candidate or test compounds which bind to or modulate the expression or activity level of, one or more biomarkers of the invention, including one or more biomarkers listed or described herein, or a fragment thereof. Such compounds include, without limitation, antibodies, proteins, fusion proteins, nucleic acid molecules, and small molecules.

In one embodiment, an assay is a cell-based assay, comprising contacting a cell expressing one or more biomarkers of the invention, including one or more biomarkers listed or described herein, or a fragment thereof, with a test compound and determining the ability of the test compound to modulate (e.g., stimulate or inhibit) the level of interaction between the biomarker and its natural binding partners as measured by direct binding or by measuring a parameter of cancer.

For example, in a direct binding assay, the biomarker polypeptide, a binding partner polypeptide of the biomarker, or a fragment(s) thereof, can be coupled with a radioisotope or enzymatic label such that binding of the biomarker polypeptide or a fragment thereof to its natural binding partner(s) or a fragment(s) thereof can be determined by detecting the labeled molecule in a complex. For example, the biomarker polypeptide, a binding partner polypeptide of the biomarker, or a fragment(s) thereof, dioisotope detected by direct counting of radioemmission or by scintillation counting. Alternatively, the polypeptides of interest a can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to modulate the interactions between one or more biomarkers of the invention, including one or more biomarkers listed or described herein, or a fragment thereof, and its natural binding partner(s) or a fragment(s) thereof, without the labeling of any of the interactants (e.g., using a microphysiometer as described in McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of the blocking agents (e.g., antibodies, fusion proteins, peptides, nucleic acid molecules, or small molecules) to antagonize the interaction between a given set of polypeptides can be accomplished by determining the activity of one or more members of the set of interacting molecules. For example, the activity of one or more biomarkers of the invention, including one or more biomarkers listed or described herein, or a fragment thereof, can be determined by detecting induction of cytokine or chemokine response, detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the biomarker or a fragment thereof (e.g., modulations of biological pathways identified herein, such as modulated proliferation, apoptosis, cell cycle, and/or E2F transcription facto binding activity). Determining the ability of the blocking agent to bind to or interact with said polypeptide can be accomplished by measuring the ability of an agent to modulate immune responses, for example, by detecting changes in type and amount of cytokine secretion, changes in apoptosis or proliferation, changes in gene expression or activity associated with cellular identity, or by interfering with the ability of said polypeptide to bind to antibodies that recognize a portion thereof.

In yet another embodiment, an assay of the present invention is a cell-free assay in which one or more biomarkers of the invention, including one or more biomarkers listed or described herein or a fragment thereof, e.g., a biologically active fragment thereof, is contacted with a test compound, and the ability of the test compound to bind to the polypeptide, or biologically active portion thereof, is determined. Binding of the test compound to the biomarker or a fragment thereof, can be determined either directly or indirectly as described above. Determining the ability of the biomarker or a fragment thereof to bind to its natural binding partner(s) or a fragment(s) thereof can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological polypeptides. One or more biomarkers polypeptide or a fragment thereof can be immobilized on a BIAcore chip and multiple agents, e.g., blocking antibodies, fusion proteins, peptides, or small molecules, can be tested for binding to the immobilized biomarker polypeptide or fragment thereof. An example of using the BIA technology is described by Fitz et al. (1997) Oncogene 15:613.

The cell-free assays of the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-114, Thesit™, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either the biomarker polypeptide, the natural binding partner(s) polypeptide of the biomarker, or fragments thereof, to facilitate separation of complexed from uncomplexed forms of one or both of the proteins, as well as to accommodate automation of the assay. Binding of a test compound in the assay can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-base fusion proteins, can be adsorbed onto glutathione Sepharose (registered trademark) beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

In an alternative embodiment, determining the ability of the test compound to modulate the activity of one or more biomarkers of the invention, including one or more biomarkers listed or described herein, or a fragment thereof, or of natural binding partner(s) thereof can be accomplished by determining the ability of the test compound to modulate the expression or activity of a gene, e.g., nucleic acid, or gene product, e.g., polypeptide, that functions downstream of the interaction. For example, inflammation (e.g., cytokine and chemokine) responses can be determined, the activity of the interactor polypeptide on an appropriate target can be determined, or the binding of the interactor to an appropriate target can be determined as previously described.

In another embodiment, modulators of one or more biomarkers of the invention, including one or more biomarkers listed or described herein, or a fragment thereof, are identified in a method wherein a cell is contacted with a candidate compound and the expression or activity level of the biomarker is determined. The level of expression of biomarker mRNA or polypeptide or fragments thereof in the presence of the candidate compound is compared to the level of expression of biomarker mRNA or polypeptide or fragments thereof in the absence of the candidate compound. The candidate compound can then be identified as a modulator of biomarker expression based on this comparison. For example, when expression of biomarker mRNA or polypeptide or fragments thereof is greater (statistically significantly greater) in the presence of the candidate compound than in its absence, the candidate compound is identified as a stimulator of biomarker expression. Alternatively, when expression of biomarker mRNA or polypeptide or fragments thereof is reduced (statistically significantly less) in the presence of the candidate compound than in its absence, the candidate compound is identified as an inhibitor of biomarker expression. The expression level of biomarker mRNA or polypeptide or fragments thereof in the cells can be determined by methods described herein for detecting biomarker mRNA or polypeptide or fragments thereof.

In yet another aspect of the invention, a biomarker of the invention, including one or more biomarkers listed or described herein, or a fragment thereof, can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300 (incorporated by reference)), to identify other polypeptides which bind to or interact with the biomarker or fragments thereof and are involved in activity of the biomarkers. Such biomarker-binding proteins are also likely to be involved in the propagation of signals by the biomarker polypeptides or biomarker natural binding partner(s) as, for example, downstream elements of one or more biomarkers-mediated signaling pathway.

The two-hybrid system is based on the modular nature of most transcription factors, which consist of separable DNA-binding and activation domains. Briefly, the assay utilizes two different DNA constructs. In one construct, the gene that codes for one or more biomarkers polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming one or more biomarkers-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with one or more biomarkers polypeptide of the invention, including one or more biomarkers listed or described herein or a fragment thereof.

In another aspect, the invention pertains to a combination of two or more of the assays described herein. For example, a modulating agent can be identified using a cell-based or a cell-free assay, and the ability of the agent to modulate the activity of one or more biomarkers polypeptide or a fragment thereof can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

IV. Uses and Methods of the Invention

The biomarkers of the invention described herein, including the biomarkers listed or described herein or fragments thereof, can be used in one or more of the following methods: a) screening assays; b) predictive medicine (e.g., diagnostic assays, prognostic assays, and monitoring of clinical trials); and c) methods of treatment (e.g., therapeutic and prophylactic, e.g., by up- or down-modulating the copy number, level of expression, and/or level of activity of the one or more biomarkers).

The isolated nucleic acid molecules of the invention can be used, for example, to (a) express one or more biomarkers of the invention, including one or more biomarkers listed or described herein or a fragment thereof (e.g., via a recombinant expression vector in a host cell in gene therapy applications or synthetic nucleic acid molecule), (b) detect biomarker mRNA or a fragment thereof (e.g., in a biological sample) or a genetic alteration in one or more biomarkers gene, and/or (c) modulate biomarker activity, as described further below. The biomarker polypeptides or fragments thereof can be used to treat conditions or disorders characterized by insufficient or excessive production of one or more biomarkers polypeptide or fragment thereof or production of biomarker polypeptide inhibitors. In addition, the biomarker polypeptides or fragments thereof can be used to screen for naturally occurring biomarker binding partner(s), to screen for drugs or compounds which modulate biomarker activity, as well as to treat conditions or disorders characterized by insufficient or excessive production of biomarker polypeptide or a fragment thereof or production of biomarker polypeptide forms which have decreased, aberrant or unwanted activity compared to biomarker wild-type polypeptides or fragments thereof (e.g., cancers, including lymphoid cancers, such as leukemia).

A. Screening Assays

In one aspect, the present invention relates to a method for preventing in a subject, a disease or condition associated with an unwanted, more than desirable, or less than desirable, expression and/or activity of one or more biomarkers described herein. Subjects at risk for a disease that would benefit from treatment with the claimed agents or methods can be identified, for example, by any one or combination of diagnostic or prognostic assays known in the art and described herein (see, for example, agents and assays described in III. Methods of Selecting Agents and Compositions).

B. Predictive Medicine

The present invention also pertains to the field of predictive medicine in which diagnostic assays, prognostic assays, and monitoring of clinical trials are used for prognostic (predictive) purposes to thereby treat an individual prophylactically. Accordingly, one aspect of the present invention relates to diagnostic assays for determining the expression and/or activity level of biomarkers of the invention, including biomarkers listed or described herein or fragments thereof, in the context of a biological sample (e.g., blood, serum, cells, or tissue) to thereby determine whether an individual is afflicted with a disease or disorder, or is at risk of developing a disorder, associated with aberrant or unwanted biomarker expression or activity. The present invention also provides for prognostic (or predictive) assays for determining whether an individual is at risk of developing a disorder associated with biomarker polypeptide, nucleic acid expression or activity. For example, mutations in one or more biomarkers gene can be assayed in a biological sample.

Such assays can be used for prognostic or predictive purpose to thereby prophylactically treat an individual prior to the onset of a disorder characterized by or associated with biomarker polypeptide, nucleic acid expression or activity.

Another aspect of the invention pertains to monitoring the influence of agents (e.g., drugs, compounds, and small nucleic acid-based molecules) on the expression or activity of biomarkers of the invention, including biomarkers listed or described herein, or fragments thereof, in clinical trials. These and other agents are described in further detail in the following sections.

1. Diagnostic Assays

The present invention provides, in part, methods, systems, and code for accurately classifying whether a biological sample is associated with a cancer or a clinical subtype thereof (e.g., lymphoid cancers, such as leukemia). In some embodiments, the present invention is useful for classifying a sample (e.g., from a subject) as a cancer sample using a statistical algorithm and/or empirical data (e.g., the presence or level of one or biomarkers described herein).

An exemplary method for detecting the level of expression or activity of one or more biomarkers of the invention, including one or more biomarkers listed or described herein or fragments thereof, and thus useful for classifying whether a sample is associated with cancer or a clinical subtype thereof (e.g., lymphoid cancers, such as leukemia), involves obtaining a biological sample from a test subject and contacting the biological sample with a compound or an agent capable of detecting the biomarker (e.g., polypeptide or nucleic acid that encodes the biomarker or fragments thereof) such that the level of expression or activity of the biomarker is detected in the biological sample. In some embodiments, the presence or level of at least one, two, three, four, five, six, seven, eight, nine, ten, fifty, hundree, or more biomarkers of the invention are determined in the individual's sample. In certain instances, the statistical algorithm is a single learning statistical classifier system. Exemplary statistical analyses are presented in the Examples and can be used in certain embodiments. In other embodiments, a single learning statistical classifier system can be used to classify a sample as a cancer sample, a cancer subtype sample, or a non-cancer sample based upon a prediction or probability value and the presence or level of one or more biomarkers described herein. The use of a single learning statistical classifier system typically classifies the sample as a cancer sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the cancer classification results to a clinician, e.g., an oncologist or hematologist.

In another embodiment, the method of the present invention further provides a diagnosis in the form of a probability that the individual has a cancer or a clinical subtype thereof. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having cancer or a clinical subtype thereof. In yet another embodiment, the method of the present invention further provides a prognosis of cancer in the individual. For example, the prognosis can be surgery, development of a clinical subtype of the cancer (e.g., subtype of leukemia), development of one or more symptoms, development of malignant cancer, or recovery from the disease. In some instances, the method of classifying a sample as a cancer sample is further based on the symptoms (e.g., clinical factors) of the individual from which the sample is obtained. The symptoms or group of symptoms can be, for example, those associated with the IPI. In some embodiments, the diagnosis of an individual as having cancer or a clinical subtype thereof is followed by administering to the individual a therapeutically effective amount of a drug useful for treating one or more symptoms associated with cancer or the cancer.

In some embodiments, an agent for detecting biomarker mRNA, genomic DNA, or fragments thereof is a labeled nucleic acid probe capable of hybridizing to biomarker mRNA, genomic DNA., or fragments thereof. The nucleic acid probe can be, for example, full-length biomarker nucleic acid, or a portion thereof, such as an oligonucleotide of at least 15, 30, 50, 100, 250 or 500 nucleotides in length and sufficient to specifically hybridize under stringent conditions well known to a skilled artisan to biomarker mRNA or genomic DNA. Other suitable probes for use in the diagnostic assays of the invention are described herein.

A preferred agent for detecting one or more biomarkers listed or described herein or a fragment thereof is an antibody capable of binding to the biomarker, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells, and fluids present within a subject. That is, the detection method of the invention can be used to detect biomarker mRNA, polypeptide, genomic DNA, or fragments thereof, in a biological sample in vitro as well as in vivo. For example, in vitro techniques for detection of biomarker mRNA or a fragment thereof include Northern hybridizations and in situ hybridizations. In vitro techniques for detection of biomarker polypeptide include enzyme linked immunosorbent assays (ELISAs), Western blots, immunoprecipitations and immunofluorescence. In vitro techniques for detection of biomarker genomic DNA or a fragment thereof include Southern hybridizations. Furthermore, in vivo techniques for detection of one or more biomarkers polypeptide or a fragment thereof include introducing into a subject a labeled anti-biomarker antibody. For example, the antibody can be labeled with a radioactive marker whose presence and location in a subject can be detected by standard imaging techniques.

In one embodiment, the biological sample contains polypeptide molecules from the test subject. Alternatively, the biological sample can contain mRNA molecules from the test subject or genomic DNA molecules from the test subject. A preferred biological sample is a hematological tissue (e.g., a sample comprising blood, plasma, B cell, bone marrow, etc.) sample isolated by conventional means from a subject.

In another embodiment, the methods further involve obtaining a control biological sample from a control subject, contacting the control sample with a compound or agent capable of detecting polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof of one or more biomarkers listed or described herein such that the presence of biomarker polypeptide, mRNA, genomic DNA, or fragments thereof, is detected in the biological sample, and comparing the presence of biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof in the control sample with the presence of biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof in the test sample.

The invention also encompasses kits for detecting the presence of a polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, of one or more biomarkers listed or described herein in a biological sample. For example, the kit can comprise a labeled compound or agent capable of detecting one or more biomarkers polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, in a biological sample; means for determining the amount of the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof,f in the sample; and means for comparing the amount of the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, in the sample with a standard. The compound or agent can be packaged in a suitable container. The kit can further comprise instructions for using the kit to detect the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof.

In some embodiments, therapies tailored to treat stratified patient populations based on the described diagnostic assays are further administered.

2. Prognostic Assays

The diagnostic methods described herein can furthermore be utilized to identify subjects having or at risk of developing a disease or disorder associated with aberrant expression or activity of one or more biomarkers of the invention, including one or more biomarkers listed or described herein, or a fragment thereof. As used herein, the term “aberrant” includes biomarker expression or activity levels which deviates from the normal expression or activity in a control.

The assays described herein, such as the preceding diagnostic assays or the following assays, can be utilized to identify a subject having or at risk of developing a disorder associated with a misregulation of biomarker activity or expression, such as in a cancer (e.g., lymphoid cancers, such as leukemia). Alternatively, the prognostic assays can be utilized to identify a subject having or at risk for developing a disorder associated with a misregulation of biomarker activity or expression. Thus, the present invention provides a method for identifying and/or classifying a disease associated with aberrant expression or activity of one or more biomarkers of the invention, including one or more biomarkers listed or described herein, or a fragment thereof. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with aberrant biomarker expression or activity. For example, such methods can be used to determine whether a subject can be effectively treated with an agent for a cancer (e.g., lymphoid cancers, such as leukemia). Thus, the present invention provides methods for determining whether a subject can be effectively treated with an agent for a disease associated with aberrant biomarker expression or activity in which a test sample is obtained and biomarker polypeptide or nucleic acid expression or activity is detected (e.g., wherein a significant increase or decrease in biomarker polypeptide or nucleic acid expression or activity relative to a control is diagnostic for a subject that can be administered the agent to treat a disorder associated with aberrant biomarker expression or activity). In some embodiments, significant increase or decrease in biomarker expression or activity comprises at least 2 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher or lower, respectively, than the expression activity or level of the marker in a control sample.

The methods of the invention can also be used to detect genetic alterations in one or more biomarkers of the invention, including one or more biomarkers listed or described herein or a fragment thereof, thereby determining if a subject with the altered biomarker is at risk for cancer (e.g., lymphoid cancers, such as leukemia) characterized by aberrant biomarker activity or expression levels. In preferred embodiments, the methods include detecting, in a sample of cells from the subject, the presence or absence of a genetic alteration characterized by at least one alteration affecting the integrity of a gene encoding one or more biomarkers polypeptide, or the mis-expression of the biomarker. For example, such genetic alterations can be detected by ascertaining the existence of at least one of 1) a deletion of one or more nucleotides from one or more biomarkers gene, 2) an addition of one or more nucleotides to one or more biomarkers gene, 3) a substitution of one or more nucleotides of one or more biomarkers gene, 4) a chromosomal rearrangement of one or more biomarkers gene, 5) an alteration in the level of a messenger RNA transcript of one or more biomarkers gene, 6) aberrant modification of one or more biomarkers gene, such as of the methylation pattern of the genomic DNA, 7) the presence of a non-wild type splicing pattern of a messenger RNA transcript of one or more biomarkers gene, 8) a non-wild type level of one or more biomarkers polypeptide, 9) allelic loss of one or more biomarkers gene, and 10) inappropriate post-translational modification of one or more biomarkers polypeptide. As described herein, there are a large number of assays known in the art which can be used for detecting alterations in one or more biomarkers gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in one or more biomarkers gene (see Abravaya et al. (1995) Nucleic Acids Res. 23:675-682). This method can include the steps of collecting a sample of cells from a subject, isolating nucleic acid (e.g., genomic DNA, mRNA, cDNA, small RNA, mature miRNA, pre-miRNA, pri-miRNA, miRNA*, anti-miRNA, or a miRNA binding site, or a variant thereof) from the cells of the sample, contacting the nucleic acid sample with one or more primers which specifically hybridize to one or more biomarkers gene of the invention, including the biomarker genes listed or described herein, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more biomarkers gene of the invention, including one or more biomarkers listed or described herein, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531 (incorporated by reference)) can be used to score for the presence of specific mutations by development or loss of a ribozyme cleavage site.

In other embodiments, genetic mutations in one or more biomarkers gene of the invention, including a gene listed or described herein, or a fragment thereof, can be identified by hybridizing a sample and control nucleic acids, e.g., DNA, RNA, mRNA, small RNA, cDNA, mature miRNA, pre-miRNA, pri-miRNA, miRNA*, anti-miRNA, or a miRNA binding site, or a variant thereof, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in one or more biomarkers can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence one or more biomarkers gene of the invention, including a gene listed or described herein, or a fragment thereof, and detect mutations by comparing the sequence of the sample biomarker gene with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101 (incorporated by reference); Cohen et al. (1996) Adv. Chromatogr. 36:127-162; and Griffin et al. (1993) Appl. Biochem. Biotechnol. 38:147-159).

Other methods for detecting mutations in one or more biomarkers gene of the invention, including a gene listed or described herein, or fragments thereof, include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker genes of the invention, including genes listed or described herein, or fragments thereof, obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). The duplex is treated with a DNA mismatch repair enzyme, and the cleavage products, if any, can be detected from electrophoresis protocols or the like. See, for example, U.S. Pat. No. 5,459,039 (incorporated by reference).

In other embodiments, alterations in electrophoretic mobility will be used to identify mutations in biomarker genes of the invention, including genes listed or described herein, or fragments thereof. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA. In some embodiments, the hybridization reactions can occur using biochips, microarrays, etc., or other array technology that are well known in the art.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose patients exhibiting symptoms or family history of a disease or illness involving one or more biomarkers of the invention, including one or more biomarkers listed or described herein, or fragments thereof.

3. Monitoring of Effects During Clinical Trials

Monitoring the influence of agents (e.g., drugs) on the expression or activity of one or more biomarkers of the invention, including one or more biomarkers listed or described herein, or a fragment thereof (e.g., the modulation of a cancer state) can be applied not only in basic drug screening, but also in clinical trials. For example, the effectiveness of an agent determined by a screening assay as described herein to increase expression and/or activity of one or more biomarkers of the invention, including one or more biomarkers listed or described herein or a fragment thereof, can be monitored in clinical trials of subjects exhibiting decreased expression and/or activity of one or more biomarkers of the invention, including one or more biomarkers of the invention, including one or more biomarkers listed or described herein, or a fragment thereof, relative to a control reference. Alternatively, the effectiveness of an agent determined by a screening assay to decrease expression and/or activity of one or more biomarkers of the invention, including one or more biomarkers listed or described herein, or a fragment thereof, can be monitored in clinical trials of subjects exhibiting decreased expression and/or activity of the biomarker of the invention, including one or more biomarkers listed or described herein or a fragment thereof relative to a control reference. In such clinical trials, the expression and/or activity of the biomarker can be used as a “read out” or marker of the phenotype of a particular cell.

In some embodiments, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting the level of expression and/or activity of one or more biomarkers of the invention, including one or more biomarkers listed or described herein or fragments thereof in the preadministration sample; (iii) obtaining one or more post-administration samples from the subject; (iv) detecting the level of expression or activity of the biomarker in the post-administration samples; (v) comparing the level of expression or activity of the biomarker or fragments thereof in the pre-administration sample with the that of the biomarker in the post administration sample or samples; and (vi) altering the administration of the agent to the subject accordingly. For example, increased administration of the agent may be desirable to increase the expression or activity of one or more biomarkers to higher levels than detected (e.g., to increase the effectiveness of the agent.) Alternatively, decreased administration of the agent may be desirable to decrease expression or activity of the biomarker to lower levels than detected (e.g., to decrease the effectiveness of the agent). According to such an embodiment, biomarker expression or activity may be used as an indicator of the effectiveness of an agent, even in the absence of an observable phenotypic response.

C. Methods of Treatment

The present invention provides for both prophylactic and therapeutic methods of treating a subject at risk of (or susceptible to) a disorder characterized by insufficient or excessive production of biomarkers of the invention, including biomarkers listed or described herein or fragments thereof, which have aberrant expression or activity compared to a control. Moreover, agents of the invention described herein can be used to detect and isolate the biomarkers or fragments thereof, regulate the bioavailability of the biomarkers or fragments thereof, and modulate biomarker expression levels or activity.

1. Prophylactic Methods

In one aspect, the invention provides a method for preventing in a subject, a disease or condition associated with an aberrant expression or activity of one or more biomarkers of the invention, including one or more biomarkers listed or described herein or a fragment thereof, by administering to the subject an agent which modulates biomarker expression or at least one activity of the biomarker. Subjects at risk for a disease or disorder which is caused or contributed to by aberrant biomarker expression or activity can be identified by, for example, any or a combination of diagnostic or prognostic assays as described herein. Administration of a prophylactic agent can occur prior to the manifestation of symptoms characteristic of the biomarker expression or activity aberrancy, such that a disease or disorder is prevented or, alternatively, delayed in its progression.

2. Therapeutic Methods

Another aspect of the invention pertains to methods of modulating the expression or activity or interaction with natural binding partner(s) of one or more biomarkers of the invention, including one or more biomarkers listed or described herein or fragments thereof, for therapeutic purposes. The biomarkers of the invention have been demonstrated to correlate with cancer (e.g., lymphoid cancers, such as leukemia). Accordingly, the activity and/or expression of the biomarker, as well as the interaction between one or more biomarkers or a fragment thereof and its natural binding partner(s) or a fragment(s) thereof can be modulated in order to modulate the immune response. In some embodiments, subjects stratified on the basis of mutant RAC biomarker expression can specifically be treated with inhibitors of PAK1, ROCK1, and/or ROCK2, either alone or in combination with other anti-cancer therapeutics, since such downstream RAC signaling molecules have been demosntrated herein to be important for the transforming ability of mutant RAC biomarkers.

Modulatory methods of the invention involve contacting a cell with one or more biomarkers of the invention, including one or more biomarkers of the invention, including one or more biomarkers listed or described herein or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, one or more biomarkers agonist or antagonist, a peptidomimetic of one or more biomarkers agonist or antagonist, one or more biomarkers peptidomimetic, other small molecule, or small RNA directed against or a mimic of one or more biomarkers nucleic acid gene expression product.

An agent that modulates the expression of one or more biomarkers of the invention, including one or more biomarkers of the invention, including one or more biomarkers listed or described herein or a fragment thereof is, e.g., an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule, triplex oligonucleotide, ribozyme, or recombinant vector for expression of one or more biomarkers polypeptide. For example, an oligonucleotide complementary to the area around one or more biomarkers polypeptide translation initiation site can be synthesized. One or more antisense oligonucleotides can be added to cell media, typically at 200 microgram/mL, or administered to a patient to prevent the synthesis of one or more biomarkers polypeptide. The antisense oligonucleotide is taken up by cells and hybridizes to one or more biomarkers mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, preferably, such modulation occurs by a means other than by knocking out the biomarker gene.

Agents which modulate expression, by virtue of the fact that they control the amount of biomarker in a cell, also modulate the total amount of biomarker activity in a cell.

In one embodiment, the agent stimulates one or more activities of one or more biomarkers of the invention, including one or more biomarkers listed or described herein or a fragment thereof. Examples of such stimulatory agents include active biomarker polypeptide or a fragment thereof and a nucleic acid molecule encoding the biomarker or a fragment thereof that has been introduced into the cell (e.g., cDNA, mRNA, shRNAs, siRNAs, small RNAs, mature miRNA, pre-miRNA, pri-miRNA, miRNA*, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, antibiomarker antibodies, biomarker inhibitors, and compounds identified in the screening assays described herein.

These modulatory methods can be performed in vitro (e.g., by contacting the cell with the agent) or, alternatively, by contacting an agent with cells in vivo (e.g., by administering the agent to a subject). As such, the present invention provides methods of treating an individual afflicted with a condition or disorder that would benefit from up- or down-modulation of one or more biomarkers of the invention listed or described herein or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering one or more biomarkers polypeptide or nucleic acid molecule as therapy to compensate for reduced, aberrant, or unwanted biomarker expression or activity.

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In addition, these modulatory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer (e.g., lymphoid cancers, such as leukemia), being treated, the extent of the disease and other factors familiar to the physician of skill in the art and can be determined by the physician.

V. Pharmaceutical Compositions

In another aspect, the present invention provides pharmaceutically acceptable compositions which comprise a therapeutically-effective amount of an agent that decreases the activity or levels of a biomarker of the present invention (e.g., a mutant RAC protein), formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that decreases the activity or levels of a biomarker of the present invention (e.g., a mutant RAC protein)x, or composition comprising an agent that decreases the activity or levels of a biomarker of the present invention (e.g., a mutant RAC protein), which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that decreases the activity or levels of a biomarker of the present invention (e.g., a mutant RAC protein). These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that decreases the activity or levels of a biomarker of the present invention (e.g., a mutant RAC protein). These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred per cent, this amount will range from about 1% to about 99% of active ingredient, preferably from about 5% to about 70%, most preferably from about 10% to about 30%.

Methods of preparing these formulations or compositions include the step of bringing into association an agent that decreases the activity or levels of a biomarker of the present invention (e.g., a mutant RAC protein), with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

### Example 1

### Material and Methods for Examples 2-6

A. Cell Lines

Human embryonic kidney 293T (HEK293T), a fibrosarcoma cell line HT1080, breast cancer cell lines HCC1143 and MDA-MB-157, and a mouse fibroblast cell line 3T3 were purchased from American Type Culture Collection (ATCC, Manassas, Va., USA) and were maintained in Dulbecco's modified Eagle's medium-F12 (DMEM/F12) (Invitrogen, Carlsbad, Calif., USA) supplemented with 10% (vol/vol) fetal bovine serum (FBS) and 2 mM L-glutamine (both from Invitrogen). The human mammary gland epithelial cell line MCF10A was obtained from ATCC and maintained in DMEM/F12 supplemented with 5% (vol/vol) horse serum (S0910, Biowest, Nuaille, France), recombinant human epidermal growth factor (EGF; 20 ng/mL) (Peprotech, Rocky Hill, N.J., USA), bovine insulin (10 microgram/mL) (1-1882, Sigma-Aldrich, St. Louis, Mo., USA), hydrocortisone (0.5 microgramg/mL, H-0888, Sigma-Aldrich) and cholera toxin (100 ng/mL, D-8052, Sigma-Aldrich). The human CML cell line KCL-22 was obtained from the Japanese Collection of Research Bioresources (Osaka, Japan), and maintained in RPMI medium 1640 (Invitrogen) supplemented with 10% (vol/vol) FBS.

B. Oligonucleotide Sequences

Primer sequences used for polymerase chain reaction (PCR) are as follows:

Ras-related C3 botulinum toxin substrate (RAC)
1 full-length cDNA:
5′-AGTTTTCCTCAGCTTTGGGTGGTG-3′
and
5′-AAAGCGTACAAAGGTTCCAAGGGA-3′;
RAC1 genome exon 2:
5′-TCAGGGTACCAATGTGTATGTGGTG-3′
and
5′-TGGTCAAAGAAATGTGAAACCCGT-3′;
RAC1 genome exon 4:
5′-CCTTCCCAGCAACATGTAGAAAGC-3′
and
5′-CAGCCTGGACACAACAGAGTGAGA-3′;
RAC2 full-length cDNA:
5′-CTGAGCTGTCACCACCGACACTCT-3′
and
5′-AGCTCTGCAGCCATCTGCTAAGAA-3′;
RAC2 genome exon 2:
5′-CTTCTACCCCTTCCTCCATACCCC-3′
and
5′-CCCTCTTGCACTTCCTGTCTTTCA-3′
RAC3 full-length cDNA:
5′-ATTTCTCCGCAGCTCGGCTC-3′
and
5′-GGACACCACACGTCTCAACACAAC-3′.

The following primers were used for site-directed mutagenesis.

RAC1(P29S):
5′-CTGATCAGTTACACAACCAATGCATTTTCTGGAGAATATATCCC-3′
and
5′-GGGATATATTCTCCAGAAAATGCATTGGTTGTGTAACTGATCAG-3′;
RAC1(C157Y):
5′-GTAAAATACCTGGAGTACTCGGCGCTCACACAG-3′
and
5′-CTGTGTGAGCGCCGAGTACTCCAGGTATTTTAC-3′;
RAC1(P179L):
5′-GCAGTCCTCTGCCTGCCTCCCGTGAAG-3′
and
5′-CTTCACGGGAGGCAGGCAGAGGACTGC-3′
RAC2(I21M):
5′-GCAAGACCTGCCTTCTCATGAGCTACACCACCAAC-3′
and
5′-GTTGGTGGTGTAGCTCATGAGAAGGCAGGTCTTGC-3′
RAC2(P29Q):
5′-CACCACCAACGCCTTTCAAGGAGAGTACATCCCCAC-3′
and
5′-GTGGGGATGTACTCTCCTTGAAAGGCGTTGGTGGTG-3′
RAC2(D47Y):
5′-CAGCCAATGTGATGGTGTACAGCAAGCCAGTGAAC-3′
and
5′-GTTCACTGGCTTGCTGTACACCATCACATTGGCTG-3′
RAC2(R106H):
5′-CGGCACCACTGCCACAGCACACCCATC-3′
and
5′-GATGGGTGTGCTGTGGCAGTGGTGCCG-3′.

The following primers were used for real-time RT-PCR:

RAC1:
5′-AAGCTGACTCCCATCACCTATCCG-3′
and
5′-CGAGGGGCTGAGACATTTACAACA- 3′;
RAC2:
5′-AAGAAGCTGGCTCCCATCACCTAC- 3′
and
5′-AACACGGTTTTCAGGCCTCTCTG- 3′;
RAC3:
5′-AAGAAGCTGGCACCCATCACCTAC- 3′
and
5′-ATCGCCTCGTCAAACACTGTCTTC-3′;
NRAS:
5′-TCAACAGCAGTGATGATGGGACTC-3′
and
5′-AGGGTGTCAGTGCAGCTTGAAAGT- 3′;
KRAS:
5′-TGGGGAGGGCTTTCTTTGTGTATT- 3′
and
5′-TGCTAAGTCCTGAGCCTGTTTTGTG- 3′;
HRAS:
5′-AGCAGATCAAACGGGTGAAGGACT- 3′
and
5′-GATCTCACGCACCAACGTGTAGAA- 3′.

C. Quantitative Real-Time RT-PCR

To examine the message level of RAC1, RAC2, RAC3, NRAS, HRAS, and KRAS in HT1080, total RNA was isolated with the use of an RNeasy (registered trademark) Mini column (Qiagen) and was subjected to reverse transcription with an oligo (dT) primer and ReverTra Ace reverse transcriptase (Toyobo). The amount of specific cDNAs was quantitated by real-time PCR analysis with a QuantiTect SYBR Green (registered trademark) PCR Kit (Qiagen). The amplification protocol comprised incubations at 94 deg C for 15 s, 60 deg C for 30 s, and 72 deg C for 60 s. Incorporation of the SYBR Green (registered trademark) dye into the PCR products was monitored in real time with an ABI PRISM 7700 (registered trademark) sequence detection system (Applied Biosystems), thereby allowing determination of the threshold cycle (CT) at which exponential amplification of PCR products begins. The cDNA copy number for each gene was quantified based on the CT standard curve generated with the corresponding control cDNA.

D. cDNA Sequencing with a Next-Generation Sequencer

Custom RNA probes of 120 bases were designed to capture cDNAs of 906 human protein-coding genes (Ueno et al. (2012) Cancer Sci. 103:131-135) and were synthesized by Agilent Technologies. The captured cDNAs were obtained from HT1080 cells with the use of the SureSelect (registered trademark) Target Enrichment system (Agilent Technologies) and were subjected to deep sequencing with a Genome Analyzer (registered trademark) IIx (Illumina). The data set was screened for nonsynonymous mutations with the use of in-house computational pipeline (Ueno et al. (2012) Cancer Sci. 103:131-135). Analysis of clinical specimens was approved by the ethics committees of the University of Tokyo, Jichi Medical University, Nagoya University Graduate School of Medicine, and The Cancer Institute. For highthroughput, deep sequencing of selected RAC1, RAC2, and RAC3 cDNAs, full-length cDNAs for each RAC protein were amplified by RT-PCR from cell lines and were subjected to Sanger sequencing to screen for nonsynonymous mutations.

E. Transformation Assays for RAC Mutants

The coding sequences of RAC1 and RAC2 cDNAs were amplified by RT-PCR from HT1080 and HCC1143 cells and were inserted into the retroviral plasmid pMXSires-EGFP (Clontech) for expression of RAC1 or RAC2 together with that of enhanced green fluorescent protein. Expression plasmids for RAC1(P29S), RAC1(C157Y), RAC1(P179L), RAC2(I21M), RAC2(P29Q), RAC2(D47Y), or RAC2(P106H) were generated by site-directed mutagenesis with the above plasmids. For the production of infectious virus particles, the plasmids were introduced into HEK293T cells together with amphotropic retroviral packaging plasmids (Takara Bio). Virus particles were then used to infect 3T3 and MCF10A cells, which were subsequently suspended in culture medium containing 0.4% (wt/vol) agar (SeaPlaque (registered trademark) GTG agarose; FMC) and layered on top of culture medium containing 0.53% (wt/vol) agar in six-well plates. Colonies were allowed to form for 14 d (3T3) or 20 d (MCF10A) and were then stained with crystal violet. 3T3 cells (1.0×10⁶) expressing wild-type or mutant forms of RAC1 or RAC2 were also injected subcutaneously (s.c.) into nu/nu mice for in vivo tumorigenicity assays. The mouse experiments were approved by the Institutional Animal Care and Use Committee of the University of Tokyo.

F. Activity of RAC Proteins

GTP-bound RAC proteins were detected with a pull-down assay based on a glutathione S-transferase (GST) fusion protein of PAK1-p21-binding domain (PBD) and performed with the use of a Racl/Cdc42 Activation Assay Kit (Millipore). For luciferase-based measurement of RAC-dependent signaling activity, HEK293T cells were transfected for 48 h with an expression plasmid for wild-type or mutant forms of RAC1 or RAC2 together with pGL-TK (Promega) and the SRE.L reporter plasmid, in which a modified serum response factor-responsive element was ligated to firefly luciferase cDNA (Hill et al. (1995) Cell 81:1159-1170). The activity of firefly luciferase in cell lysates was then measured and normalized by that of Renilla luciferase. For immunoblot analysis, antibodies to RAC1 (clone 23A8) and to NRAS (OP25) were obtained from Millipore, those to RAC2 (ab2244) were from Abcam, and those to ACTB (13E5) were from Cell Signaling Technology. For phalloidin staining, cells were fixed with 4% (vol/vol) paraformaldehyde in PBS for 10 min at room temperature, permeabilized for 5 min with 0.1% (vol/vol) Triton X-100 in PBS, and then exposed to Alexa Fluor 594-conjugated phalloidin V (Molecular Probes, Invitrogen) in the presence of bovine serum albumin (BSA; 10 microgram/mL). The cells were then counterstained with Hoechst 33258 and examined with a fluorescence microscope (Olympus).

G. RNA Interference

Control, RAC1, and NRAS siRNAs were obtained from Dharmacon and were introduced into HT1080 cells by transfection with the use of RNAiMAX (Invitrogen). The target sequences for RNAi were 5′-UAAGGAGAUUGGUGCUGUA-3′ (RAC1 siRNA #7 and shRNA), 5′-CGGCACCACUGUCCCAACA-3′ (RAC1 siRNA #9), 5′-AUACGCCAGUACCGAAUGA-3′ (NRAS siRNA #3), and 5′-AAAGCGCACUGACAAUCCA-3′ (NRAS siRNA #4). After transfection, cells were maintained in the culture medium containing 10% (vol/vol) FBS unless otherwise specified. Expression vectors for shRNAs were constructed with the pMK0.1 plasmid (Addgene). To generate shRNA-resistant RAC1 cDNA, the TAAGGAGATTGGTGCTGTA sequence at nucleotides 679-697 of human RAC1 cDNA (GenBank accession no. NM_{—}006908.4) was changed to CAAAGAAATTGGAGCAGTG. Such base substitutions do not affect amino acid sequence of the translated protein.

The cell cycle of HT1080 cells transfected with various siRNAs was analyzed with an FITC BrdU Flow Kit and the FACSCanto (registered trademark) II instrument (both from BD Biosciences). The enzymatic activity of CASP3/CASP7 was measured with the Casepase-Glo 3/7 (registered trademark) Assay (Promega).

Other siRNAs were purchased from Dharmacon for PAK1 (#M-003521-04-0005), ROCK1 (#M-003536-02-0005), and ROCK2 (#M-004610-02-0005).

H. Biochemical Analyses of Recombinant RAC1 Proteins

The coding cDNAs for the wild-type or the mutant forms of RAC1 corresponding to 1-188 amino acids was inserted into the pGEX6-P-1 plasmid (GE Healthcare Life Sciences). GST fusion protein of RAC1 or its mutants was induced with 0.1 mM isopropyl-1-thio-beta-D-galactopyranoside at 20 deg C for 16 h in Escherichia coli BL21-CodonPlus (registered trademark) DE3 (Stratagene) harboring the plasmid and purified with glutathione-Sepharose 4B beads (GE Healthcare Life Sciences). Each GST-fusion protein was further incubated with PreScission (registered trademark) Protease (GE Healthcare Life Sciences) and glutathione-Sepharose 4B beads for 16 h to remove GST portions. The resultant supernatant was applied to a Sephadex G-25 gel filtration column (GE Healthcare Life Sciences) equilibrated with a buffer consisting of 50 mM Tris HCl (pH 7.5), 100 mM NaCl, 5 mM MgCl2, 1 mM DTT, and 0.1% (wt/vol) Lubrol PX (Nacalai Tesque), and the fractions containing the Rac proteins were collected.

Biochemical assays for RAC1 proteins were performed as described previously in Kontani et al. (2002) J. Biol. Chem. 277:41070-41078 with slight modifications. For [³⁵S]GTPgammaS-binding assays, purified proteins (5 pmol) were incubated at 30 deg C with 5 micrometer of the radiolabeled nucleotides (7,000 cpm/pmol) in a 50-microliter solution consisting of 50 mM Tris-Cl (pH 7.5), 100 mM NaCl, 1 mM EDTA, 1.81 mM MgCl2, 1 mM DTT, 0.0008% (wt/vol) Lubrol PX, and 200 microgram/mL BSA. After incubation for various periods, samples were diluted with 1 mL of an ice-cold wash buffer [20 mM Tris-HCl (pH 7.5), 20 mM MgCl2, and 100 mM NaCl] and filtered through a nitrocellulose membrane (0.45-micrometer pore size; Advantec MFS). The membrane was washed four times with 2 mL of the ice-cold wash buffer and dried at 68 deg C. Radioactivity retained on the membrane was determined by a liquid scintillation counter.

For [³H]GDP- and [³⁵S]GTPgammaS-dissociation assays, purified proteins (5 pmol) were incubated with 5 micrometer of the radiolabeled nucleotides (2,000 dpm/pmol for [³H]GDP and 2,000 cpm/pmol for [³⁵S]GTPgammaS) for 10 min in a solution of 50 mM Tris-HCl (pH 7.5), 100 mM NaCl, 1 mM EDTA, 1.81 mM (for the C157Y mutant) or 0.1 mM (for the others) MgCl2, 1 mM DTT, 0.0007% (wt/vol) Lubrol PX, and 200 microgram/mL BSA. The high Mg²⁺ condition (final 0.8 mM) was used for preloading the C157Y mutant with the radiolabeled nucleotides because the mutant could not bind efficiently to the nucleotides under the low Mg²⁺ condition (final 45 nM). [³H]GDP or [³⁵S]GTPgammaS dissociation from the proteins was initiated by the addition of unlabeled GTPgammaS and MgCl₂ to final concentrations of 200 micrometer and 0.8 mM Mg²⁺, respectively. After incubation for various periods at 30 deg C, the amounts of [³H]GDP- or [³⁵S]GTPgammaS-bound proteins were determined using a nitrocellulose membrane as described above.

For GTPase assay, purified proteins were preloaded with 5 micrometer [gamma-³²P]GTP (4,000 cpm/pmol) in a solution of 50 mM Tris-HCl (pH 7.5), 100 mM NaCl, 1 mM EDTA, 1.81 mM (for the C157Y mutant) or 0.1 mM (for the others) MgCl2, 1 mM DTT, 0.0008% (wt/vol) Lubrol PX, and 200 microgram/mL BSA, and then GTP hydrolysis reactions were initiated by the addition of unlabeled GTP and MgCl2 to final concentrations of 1 and 0.8 mM Mg²⁺, respectively. After incubation for various periods at 30 deg C, the reaction mixtures containing 5 pmol of RAC proteins (50 microliter) were mixed with 750 microliter of ice-cold 50 mM NaH₂PO₄ and 5% (wt/vol) activated charcoal (Wako Pure Chemical Industries), and incubated on ice for 15 min. The mixtures were then centrifuged at 15,000×g for 10 min at 4 deg C, and the supernatants were analyzed with a liquid scintillation counter for the amounts of ³²Pᵢ released from the proteins.

### Example 2

### Identification of the RAC1(N92I) Oncoprotein

To identify transforming genes in the fibrosarcoma cell line HT1080 (Rasheed et al. (1974) Cancer 33:1027-1033), cDNAs for cancer-related genes (n=906) were isolated from HT1080 cells and subjected to deep sequencing with the Genome Analyzer IIx (GAIIx) system. Quality filtering of the 92,025,739 reads obtained yielded 45,325,377 unique reads that mapped to 843 (93.0%) of the 906 target genes. The mean read coverage for the 843 genes was 495× per nucleotide, and 70% or more of the captured regions for 568 genes were read at 10× or more coverage.

Screening for nonsynonymous mutations in the data set with the use of the computational pipeline according to Ueno et al. (2012) Cancer Sci 103:131-135 revealed a total of five missense mutations with a threshold of 30× or more coverage and a 30% or more mutation ratio (Table 1).

TABLE 1
Missense mutations identified in HT1080

| | | | | | Mu- |
|---|---|---|---|---|---|
| | | | Amino | | tation |
| Gene | GenBank | Nucleotide | Acid | Total | ratio |
| symbol | accession no. | change | change | coverage | (%) |
| DTX2 | NM_020892 | c 1,095 A > C | p.Y180S | 40× | 40.0 |
| EIF24K4 | NM_001013703 | c 1,587 C > G | p.Q513E | 736× | 44.6 |
| NRA5 | NM_002524 | c 4 [symbol] 5 C > A | p.Q61K | 172× | 49.4 |
| RAC1 | NM_006908 | c 516 A > T | p.N92I | 24,238× | 47.5 |
| SALL4 | NM_020436 | c 1,850 G > T | p.M583I | 34× | 100.0 |

[symbol]

indicates data missing or illegible when filed

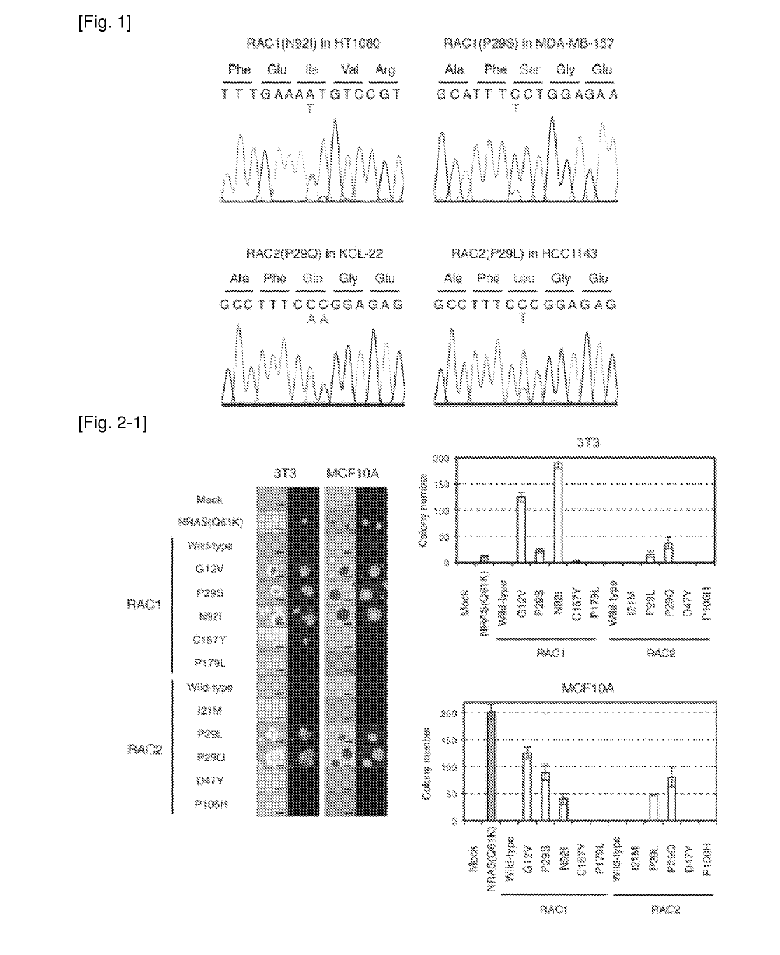

One of these mutations, a heterozygous missense mutation of NRAS (GenBank accession number, NM_{—}002524.4) that results in a Gln-to-Lys substitution at amino acid position 61 (Q61K), was described previously in this cell line (Hall et al (1983) Nature 303:396-400) and is the most frequent transforming mutation of NRAS (Cox and Der (2010) Small GTPases 1:2-27). A missense mutation in another small GTPase, RAC1, was also discovered (FIG. 1 and Table 1). An A-to-T transversion at position 516 of human RAC1 cDNA (GenBank accession no. NM_{—}006908.4), resulting in an Asn-to-Ile substitution at position 92 of the encoded protein, was thus identified in 11,525 (47.5%) of the 24,238 total reads covering this position.

To examine the transforming potential of RAC1(N92I), mouse 3T3 fibroblasts and MCF10A human mammary epithelial cells (Debnath et al. (2003) Methods 30:256-268) were infected with a retrovirus encoding wild-type or N92I mutant form of human RAC1 and the cells were then seeded in soft agar for evaluation of anchorage-independent growth. Neither 3T3 nor MCF10A cells expressing wild-type RAC1 grew in soft agar (FIG. 1A), indicating the lack of transforming potential of RAC1. In contrast, the cells expressing RAC1(N92I) readily grew in soft agar (FIG. 1A), showing that this RAC1 mutant confers the property of anchorage-independent growth on both 3T3 and MCF10A cells. The transforming potential of an artificial mutant of RAC1, RAC1(G12V) (Ridley et al. (1992) Cell 70:401-410), which harbors an amino acid substitution corresponding to that of the oncogenic G12V mutant form of RAS proteins, was also confirmed.

### Example 3

### Identification of Other Transforming Mutations of RAC1 and RAC2

Additional transforming mutations of RAC proteins were identified. cDNAS for human RAC1, RAC2 (GenBank Accession No. NM_{—}002872.3), and RAC3 (GenBank Accession No. NM_{—}005052.2) were isolated from 40 cancer cell lines (Table 2), and their nucleotide sequences were determined by Sanger sequencing, resulting in the discovery of RAC1(P29S), RAC2(P29Q), and RAC2(P29L) in the breast cancer cell line MDA-MB-157, the CML cell line KCL-22, and the breast cancer cell line HCC1143, respectively (FIG. 1 and Table 3). Further searching for RAC1, RAC2, and RAC3 mutations in the COSMIC database of cancer genome mutations (Release V59; available on the world wide web at cancer.sanger.ac.uk/cancergenome/projects/cosmic) revealed various amino acid substitutions detected in human tumors, namely RAC1(P29S), RAC1(C157Y), RAC1(P179L), RAC2(I21M), RAC2(P29L), RAC2(D47Y), and RAC2(P106H) (Table 3). All of these RAC1 and RAC2 mutations identified in clinical specimens were confirmed to be somatic, given that the corresponding mutations were absent in the genome of paired normal cells.

TABLE 2
Cell lines examined for RAC1, RAC2, and RAC3 mutations

| Cell line | Disease |
|---|---|
| BT-20 | Breast cancer |
| HCC3 [symbol] | Breast cancer |
| HCC1143 | Breast cancer |
| HCC1599 | Breast cancer |
| HCC1937 | Breast cancer |
| MDA-MB-157 | Breast cancer |
| MDA-MB-231 | Breast cancer |
| MDA-MB-436 | Breast cancer |
| MDA-MB-468 | Breast cancer |
| Hs578T | Breast cancer |
| K-562 | Chronic myeloid leukemia |
| KCL-22 | Chronic myeloid leukemia |
| KU812 | Chronic myeloid leukemia |
| MEGO1s | Chronic myeloid leukemia |
| NCO2 | Chronic myeloid leukemia |
| KYSE-520 | Esophageal squamous cell carcinoma |
| OE21 | Esophageal squamous cell carcinoma |
| 8305C | Thyroid carcinoma |
| HTOC3 | Undifferentiated thyroid carcinoma |
| T3M-1 CI-10 | Squamous cell carcinoma of the oral cavity |
| T3M-5 | Squamous cell carcinoma of the thyroid |
| Capan-2 | Pancreatic adenocarcinoma |
| Panc2.03 | Pancreatic adenocarcinoma |
| KATOIII | Gastric cancer |
| NUGC-4 | Gastric cancer |
| OCUM-1 | Gastric cancer |
| OCUM-12 | Gastric cancer |
| OCUM-2M | Gastric cancer |
| NCI-H1581 | Nonsmall cell lung cancer |
| NCI-H1703 | Nonsmall cell lung cancer |
| JHH1 | Hepatocellular carcinoma |
| JHH4 | Hepatocellular carcinoma |
| SNU-398 | Hepatocellular carcinoma |
| SNU-449 | Hepatocellular carcinoma |
| HuCCTI | Bile duct carcinoma |
| 143 [symbol] | Osteosarcoma |
| U2OS | Osteosarcoma |
| Hs633T | Fibrosarcoma |
| SW872 | Fibrosarcoma |
| SW982 | Fibrosarcoma |

[symbol]

indicates data missing or illegible when filed

TABLE 3
Missense mutations in RAC1 and RAC2

| | Nucleotide | Amino acid | Transforming | | Somatic | Public |
|---|---|---|---|---|---|---|
| | change | change | ability | Sample | mutation | database |
| RAC1 (NM_06908) | c.516 A > T | p.N92I | Yes | HT1080 cell line | Unknown | Not |
| | | | | | | deposited |
| | c.326 C > T | p.P29S | Yes | MDA-MB-157 cell line | Unknown | Not |
| | | | | | | deposited |
| | c.326 C > T | p.P29S | Yes | Mouth, squamous cell carcinoma specimen | Yes | COSMIC |
| | c.711 G > A | p.C157Y | Yes | Lung, adenocarcinoma specimen | Yes | COSMIC |
| | c.777 C > T | p.P179L | No | Skin, squamous cell carcinoma specimen | Yes | COSMIC |
| RAC2 (NM_002872) | c.180 C > G | p.I21M | No | Larynx, squamous cell carcinoma specimen | Yes | COSMIC |
| | c.203 C > T | p.P29L | Yes | HCC1143 cell line | Yes | COSMIC |
| | c.203-204 CC > AA | p.P29Q | Yes | KCL-22 cell line | Unknown | Not |
| | | | | | | deposited |
| | c.256 G > T | p.D47Y | No | Glioma specimen | Yes | COSMIC |
| | c.434 C > A | p.P106H | No | Malignant melanoma specimen | Yes | COSMIC |

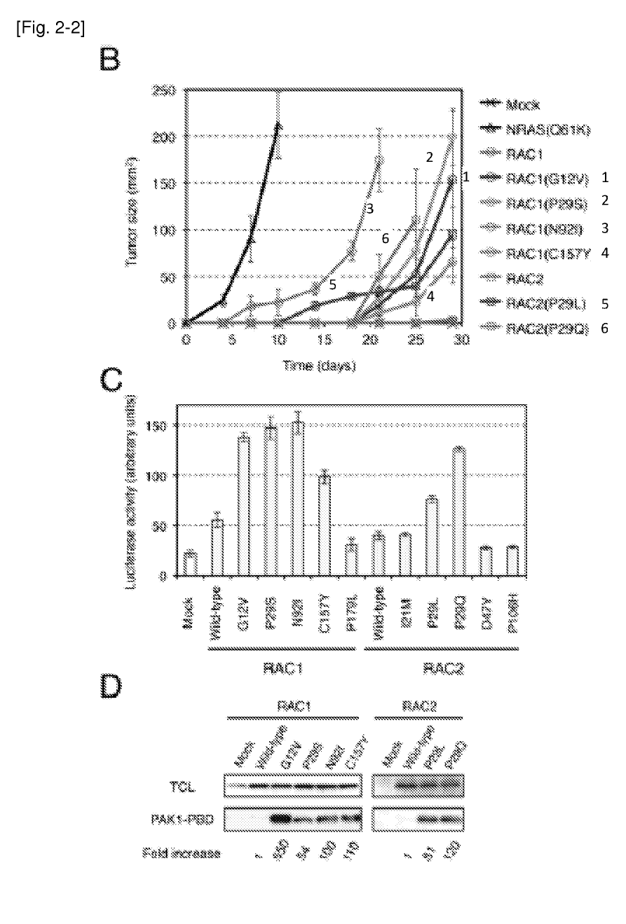

To examine the transforming potential of these various RAC1 and RAC2 mutants, each protein was expressed in 3T3 and MCF10A cells and the cells were evaluated for anchorage-independent growth. Whereas the wild-type form of RAC2 did not transform 3T3 or MCF10A cells, growth in soft agar was apparent for 3T3 cells expressing RAC1 (P29S), RAC1(C157Y), RAC2(P29L), or RAC2(P29Q), but not for those expressing RAC1(P179L), RAC2(I21M), RAC2(D47Y), or RAC2(P106H) (FIG. 2A). Of interest, colony number in the assay varied substantially in a manner dependent on the type of amino acid substitution as well as on cell type. RAC1(C157Y), for example, yielded fewer colonies in soft agar compared with the other transforming mutants. Furthermore, RAC1(P29S), which was identified in a breast cancer cell line, generated a larger number of colonies with MCF10A cells than with 3T3 cells. Conversely, RAC1(N92I), which was identified in a fibrosarcoma cell line, yielded a larger number of colonies with 3T3 cells than with MCF10A cells.

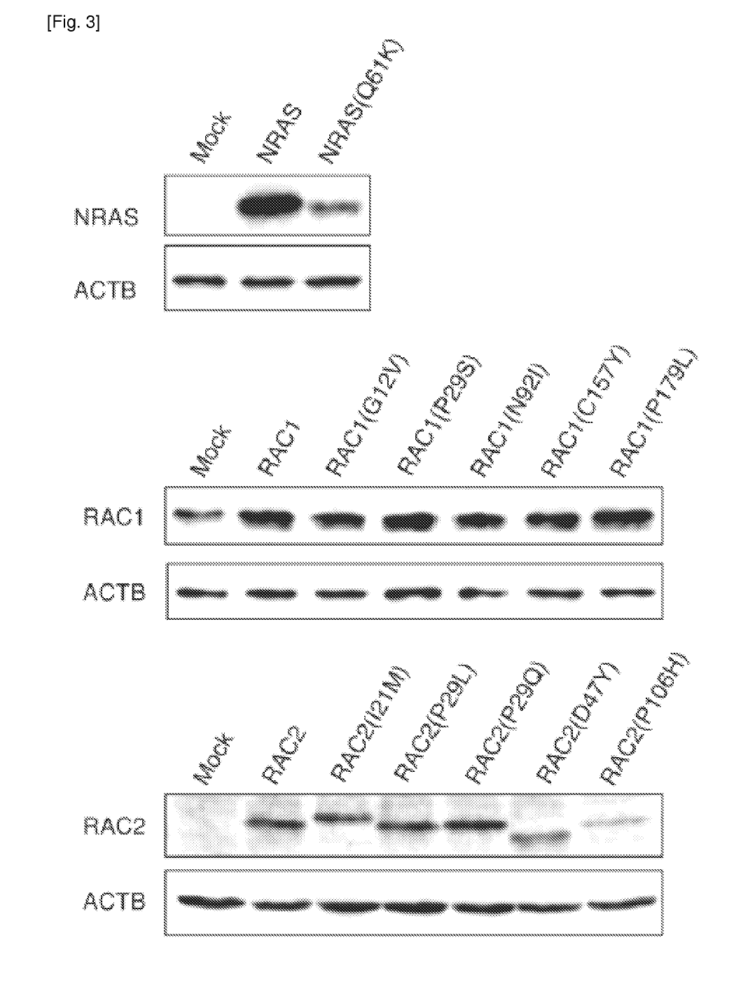

The oncogenic activity of RAC1(P29S), RAC1 (N92I), RAC1(C157Y), RAC2(P29L), and RAC2(P29Q) mutants was further confirmed with a tumorigenicity assay in nude mice (FIG. 2B), with the activity of RAC1(N92I) being the most pronounced with regard to the transformation of 3T3 cells in this assay. The colony number in soft agar for 3T3 cells expressing NRAS (Q61K) was fewer than that for the cells expressing oncogenic RAC1 or RAC2 mutants (FIG. 2A), whereas expression of these small GTPases was readily confirmed in 3T3 (FIG. 3). Subcutaneous (s.c.) tumors from the same 3T3 cells expressing NRAS(Q61K) grew more rapidly than tumors expressing the RAC1/RAC2 mutants (FIG. 2B), indicating that the measured intensity of the transforming potential of GTPases may vary in a dependent manner on assay systems.

To examine whether such oncogenic potential is linked directly to the activation of RAC1 or RAC2, the activity of the mutant proteins was evaluated using a luciferase reporter plasmid that selectively responds to intracellular signaling evoked by RHO family GTPases (Hill et al. (1995) Cell 81:1159-1170). In concordance with the data from the soft agar and tumorigenicity assays, only the transforming mutants of RAC1 and RAC2 yielded a substantial level of luciferase activity in transfected HEK293T cells (FIG. 2C).

Activated RAC1 or RAC2 would be expected to be loaded with GTP. Thus, the GTP-binding status of the RAC1 and RAC2 oncoproteins was examined with the use of a pull-down assay based on the p21-binding domain (PBD) of PAK1. All of the transforming RAC1 and RAC2 mutants were found to exist preferentially in the GTP-bound state (FIG. 2D), indicative of their constitutive activation. Furthermore, these RAC1 and RAC2 mutants induced marked reorganization of the actin cytoskeleton in 3T3 cells, resulting in the accumulation of polymerized actin in ruffles at the plasma membrane (FIG. 4).

### Example 4

### Identification of RAC1 and RAC2 as Therapeutic Targets

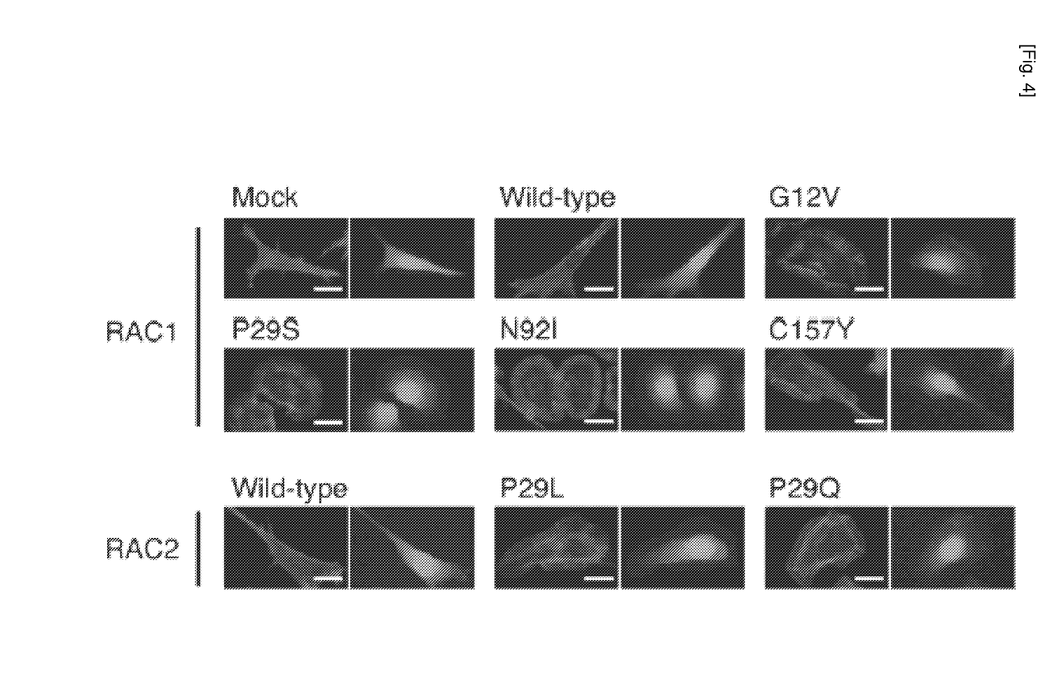

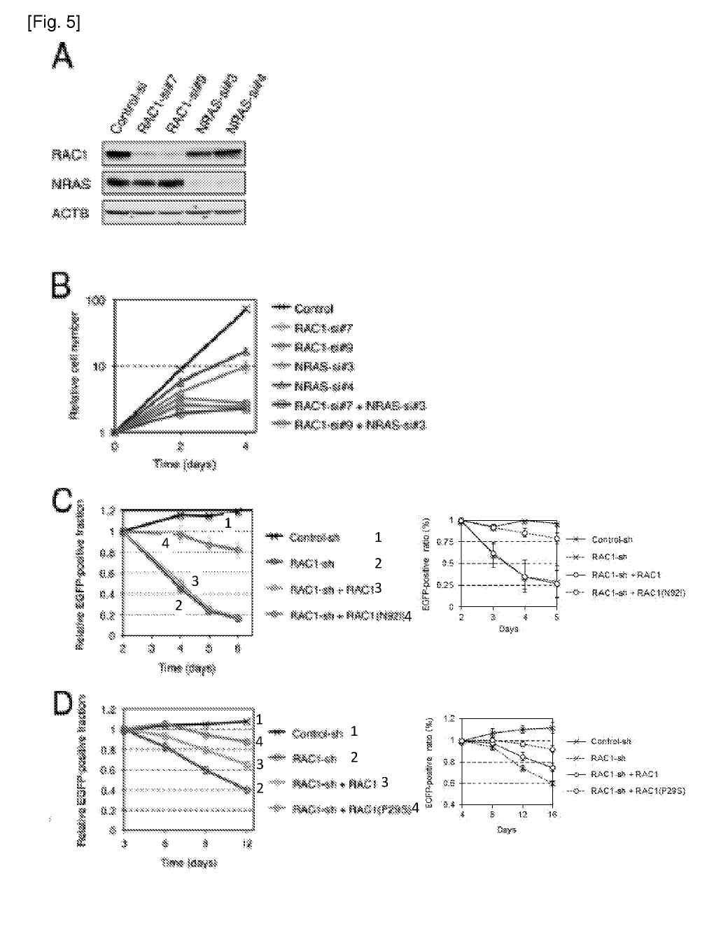

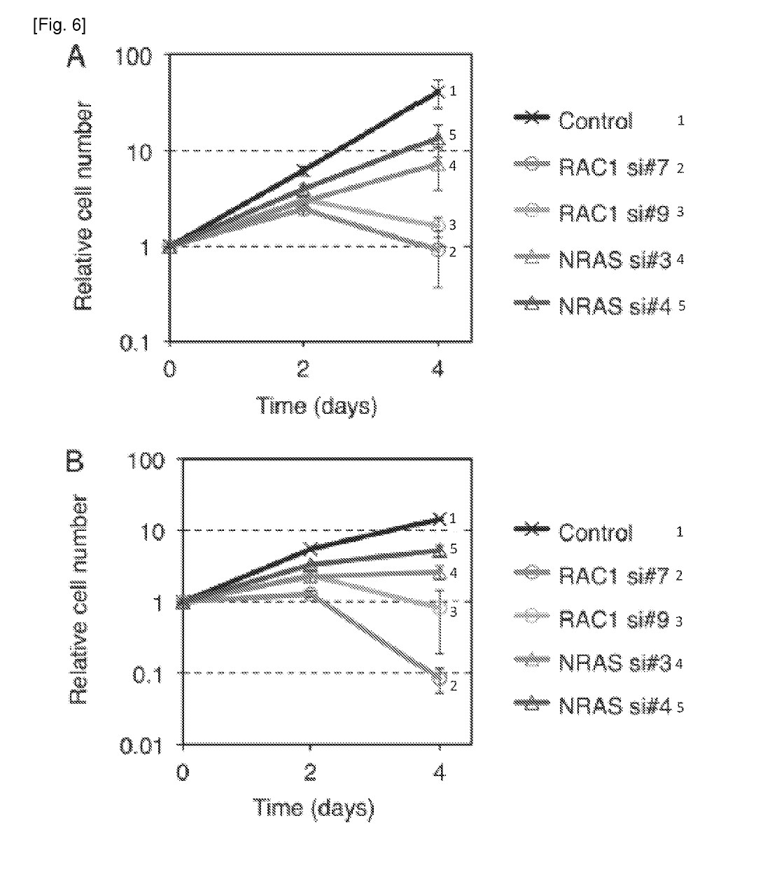

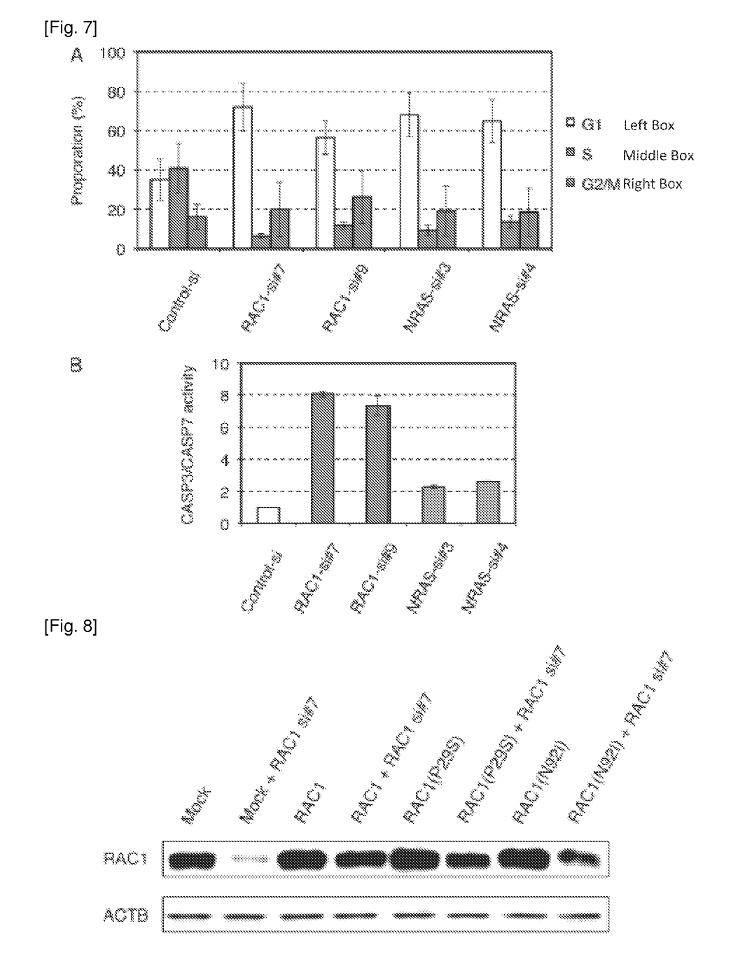

Given that NRAS(Q61K) is also known to transform 3T3 cells (FIG. 2A; Marshall et al. (1982) Nature 299:171-173), the data provided herein show that HT1080 cells harbor two independent oncogenic GTPases. Thus, it was examined whether RAC1(N92I) or NRAS(Q61K) is the principal growth driver in this sarcoma cell line. Among several small interfering RNAs (siRNAs) designed to attenuate the expression of RAC1 or NRAS, two independent siRNAs that specifically target each mRNA (FIG. 5A) were selected. Whereas transfection of HT1080 cells with either NRAS siRNA resulted in a moderate inhibition of cell proliferation under the presence of 10% (vol/vol) FBS, that with either RAC1 siRNA almost blocked cell growth (FIG. 5B). Transfection with an NRAS siRNA in addition to either RAC1 siRNA did not result in an additional effect on cell proliferation (FIG. 5B). Similar data were observed in a culture with 1% (vol/vol) FBS (FIG. 6A) or under FBS-free conditions (FIG. 6B). To further examine the effects of silencing RAC1/NRAS, cell cycle distribution of HT1080 transfected with siRNAs against either RAC1 or NRAS we quantitated. As shown in FIG. 7A, DNA synthesis was equally suppressed by the knockdown of RAC1 or NRAS. However, CASP3/CASP7 activity (a surrogate marker for apoptosis) was markedly induced only by RAC1 depletion (FIG. 7B). Therefore, RAC proteins are likely to provide RAS-independent cell survival signals, which is supported by the fact that, even under FBS-free conditions, RAC1 depletion has more antiproliferative effects in HT1080 than NRAS depletion (FIG. 7B). These data show that active RAC1 is the essential growth driver in HT1080 cells and is therefore a potential therapeutic target. Furthermore, the data indicate that oncogenic RAS proteins may require additional transforming hits to give rise to full-blown cancer.

Next, HT1080 cells were infected with a retrovirus expressing a short hairpin RNA (shRNA) targeted to RAC1 mRNA. Expression of the RAC1 shRNA markedly suppressed cell growth, whereas restoration of shRNA-resistant RAC1(N92I) expression reversed this effect (FIG. 5C and FIG. 8), showing that the effect of the RAC1 shRNA was not an off-target artifact. Forced expression of shRNA-resistant wild-type RAC1 failed to reverse the inhibitory effect of the RAC1 shRNA on cell growth, indicating that growth suppression by the shRNA was due to depletion of the N92I mutant, not to that of the wild-type protein. Similar experiments were performed with the breast cancer cell line, MDA-MB-157, which harbors RAC1(P29S). Again, the RAC1 shRNA inhibited cell growth and this effect was reversed to a larger extent by restoration of the expression of shRNA-resistant RAC1(P29S) than by forced expression of the wild-type protein (FIG. 5D and FIG. 8).

### Example 5

### RAC1(P29S), RAC1(N92I), and RAC1(C157Y) are Rapid-Cycling Mutants

Oncogenic mutations at G12, G13, or Q61 of RAS proteins found in human tumors reduce the intrinsic GTPase activity of these proteins and thereby maintain them in the GTP-bound state (Adari et al. (1988) Science 240:518-521; Cales et al. (1988) Nature 332:548-551). On the other hand, an artificial F28L substitution in HRAS or the RHO family protein Cdc42Hs was shown to confer constitutive activity by accelerating the transition from the GDP-bound to the GTP-bound state without the involvement of an exogenous guanine nucleotide exchange factor (GEF) (Reinstein et al. (1991) J. Biol. Chem. 266:17700-17706; Lin et al. (1997) Curr. Biol. 7:794-797).

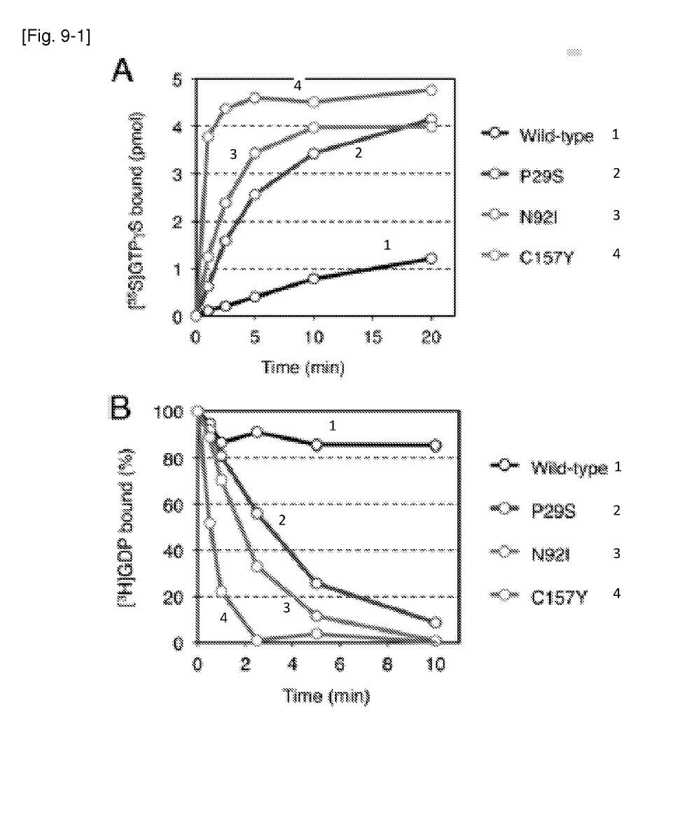

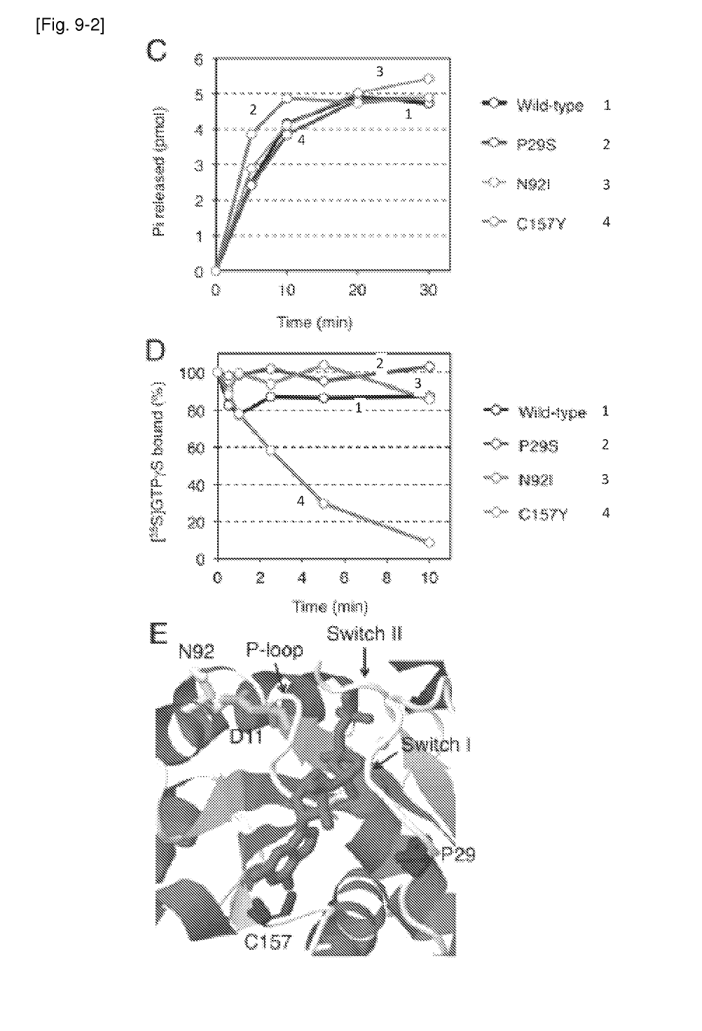

To determine how transforming mutations of RAC1 result in constitutive activation of these proteins, affinity of these mutants for GTP and GDP was examined. Compared with wild-type RAC1, all of RAC1(P29S), RAC1(N92I), and RAC1(C157Y) were found to bind GTPgammaS (nonhydrolyzable GTP analog) rapidly in vitro, even without the addition of a GEF protein (FIG. 9A). Likewise, the dissociation of GDP from the mutant forms of RAC1 was greatly accelerated (FIG. 9B). On the other hand, the intrinsic GTPase activity of these mutants was similar to (for P29S and N92I) or slightly higher (for C157Y) than that of the wild-type protein (FIG. 9C). These data thus indicated that, in contrast to transforming RAS mutants associated with human cancer, RAC1 (P29S), RAC1(N92I), and RAC1(C157Y) are fast-cycling mutants, for which the probability of being in the GTP-bound state is increased as the result of an increased rate of GDP dissociation, rather than as the result of a loss of GTPase activity.

Dissociation of GTPgammaS was also accelerated only for RAC1(C157Y), but not for the wild-type, P29S, or N92I form of RAC1 (FIG. 9D). Thus, RAC1(C157Y) is a unique mutant in that both association and dissociation for GTP are accelerated, which may provide the molecular basis for its modest transforming potential compared with that of RAC1(P29S) or RAC1 (N92I) (FIG. 2).

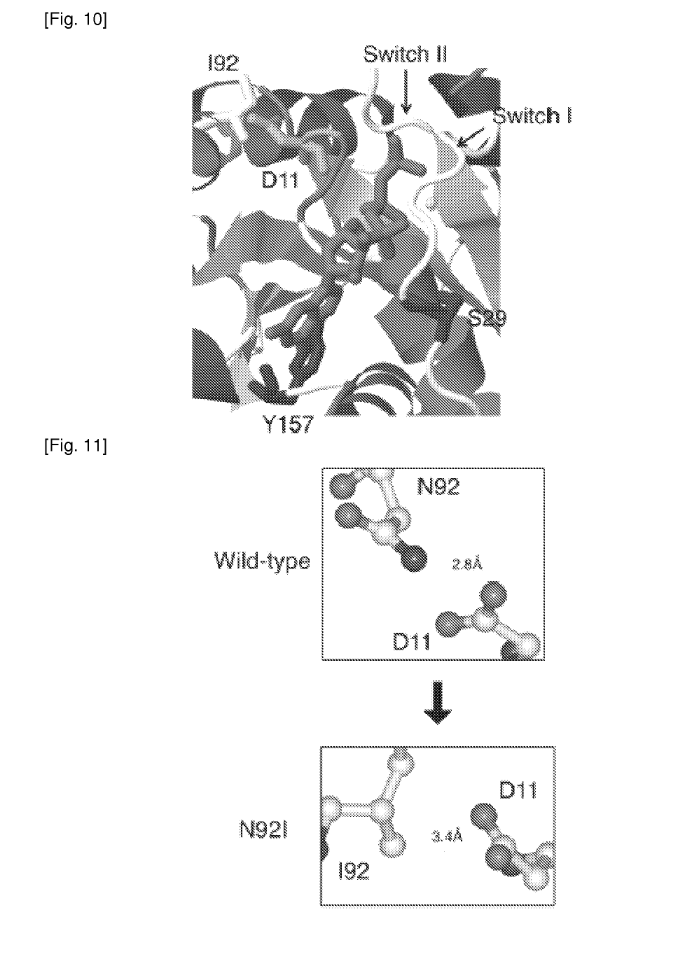

In the 3D structure of RAC1 (FIG. 7E), P29 is located in the switch I region, whereas C157 is positioned adjacent to the guanine ring of bound GTP. Substitution of these residues would thus likely affect the affinity of the protein for GDP or GTP (FIG. 10), a phenomenon that has been demonstrated recently for RAC1 (P29S) (Krauthammer et al. (2012) Nat. Genet. 44:1006-1014). In contrast, N92 is located distant from the binding pocket for GDP/GTP and so the structural mechanism by which the N92I substitution renders RAC1 constitutively active remains elusive (FIG. 9E and FIG. 10). Residue N92 is located close to D11 in the P-loop of RAC1, however (FIG. 9E and FIG. 11), and substitution with isoleucine at this position would abolish the interaction between the amino group of N92 and the carboxyl group of D11. It is thus possible that the N92I mutation affects the binding of GDP/GTP through an effect on the P-loop.

### Example 6

### Downstream Targets of RAC

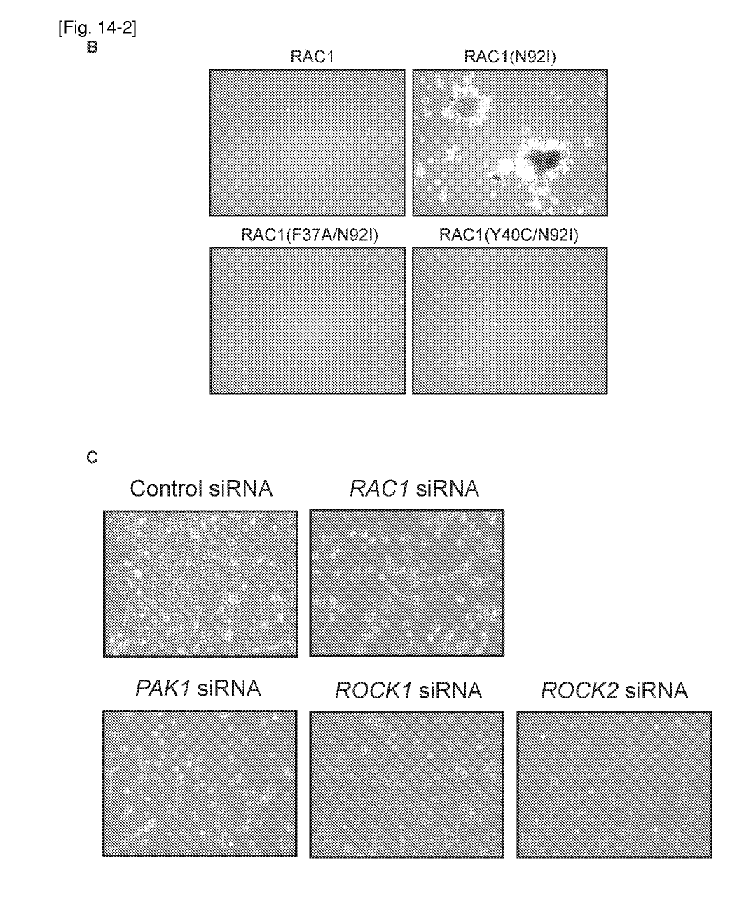

RAC protein-driven signals are known to be mediated through PAK1, ROCK1 and/or ROCK2 serine/threonine kinases. The Phe-37 or Tyr-40 residue of RAC1 plays essential roles in the binding/activation of ROCK1 or PAK1, respectively (FIG. 14A; Lamarche et al. (1996) Cell 87:519-529). Experiments were conducted to determine which pathways are indispensable for the transforming ability of the RAC1 mutants. While RAC1(N92I) confers the ability for anchorage-independent growth to 3T3 cells, substitution of Phe-37 to Ala of RAC1 almost completely abolishes such potential (FIG. 14B). Another substitution at Tyr-40 (Y40C) similarly cancels the oncogenic potential of RAC1(N92I) (FIG. 14B), indicating that both of PAK1- and ROCK1-mediated pathways are essential for the transforming signaling.

For further confirmation, the expression of PAK1 or ROCK1 was individually depleted with corresponding siRNA. Both of PAK1- and ROCK1-knock down hampered the growth of HT1080 cells harboring RAC1(N92I) (FIG. 14C). Since the growth inhibitory effect of ROCK1 siRNA was less prominent compared to that of PAK1 siRNA, another member of the ROCK family of kinases, ROCK2, was also depleted. As shown in FIG. 14C, the knockdown of ROCK2 decreased the HT1080 growth to a greater extent than that of ROCK1. These data demonstrate that all of PAK1, ROCK1 and ROCK2 kinases are suitable therapeutic targets for cancer cells with activated RAC family of proteins.

Based on the foregoing, the transforming potential of mutated RAC proteins has been demonstrated. Analysis of the described cell lines resulted in the identification of transforming mutants of RAC1 and RAC2, namely RAC1(N92I) and RAC2(P29Q), and the transforming potential of the RAC1(P29S), RAC1(C157Y), and RAC2(P29L) mutants deposited the COSMIC database of cancer genome mutations (Release V59; available on the world wide web at cancer.sanger.ac.uk/cancergenome/projects/cosmic) (Table 3). In contrast, the soft agar assay did not reveal a transforming potential of the RAC1(P179L), RAC2(I21M), RAC2(D47Y), or RAC2(P106H) mutants found in the database, suggesting a possibility that they are “passenger mutations.” It is believed, however, that these mutants still contribute to cancer development by modifying tumor properties (such as metastasis ability), given that they were somatically acquired and clonally selected in cancer.

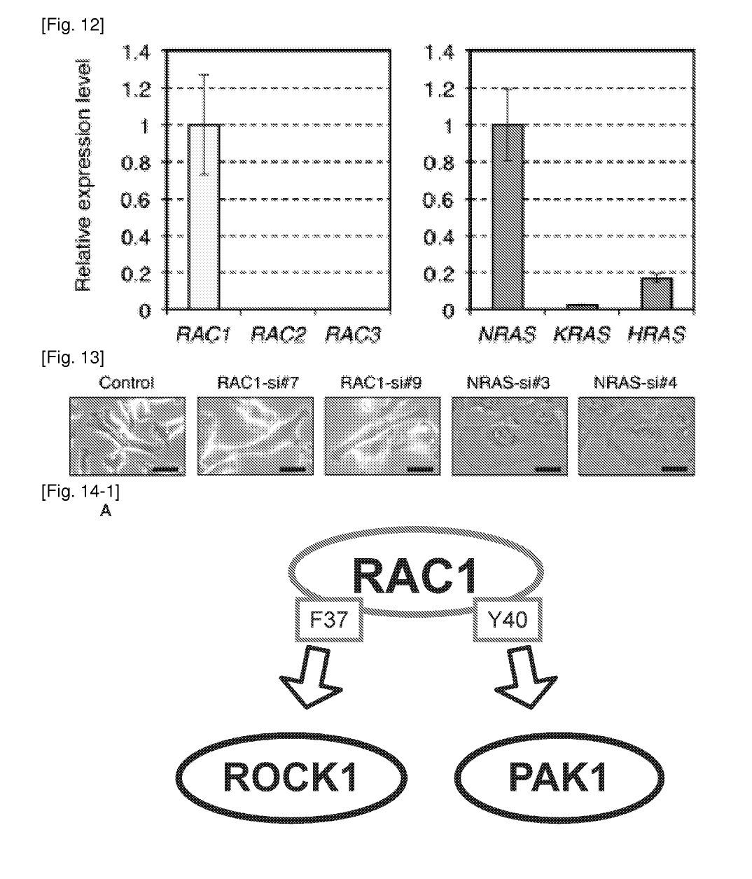

In addition, the oncogenic effects of RAC1(N92I) may be more pronounced than those of NRAS(Q61K), at least with regard to survival signals in HT1080 cells (FIG. 5B). It should be noted, however, that HT1080 expresses RAC1 almost exclusively among the RAC family proteins, whereas HRAS and KRAS are weakly expressed in addition to NRAS (FIG. 12). It is thus possible that the effects of NRAS knockdown in FIG. 5B may be partly complemented by the residual HRAS/KRAS proteins.

Paterson et al. previously isolated NRAS-attenuated subclones of HT1080 after treatment with an alkylating reagent (N-methyl-N′-nitro-N-nitrosoguanidine) and a subsequent culture with 5-fluorodeoxyuridine and 1-beta-D-arabinofuranosylcytosine (Paterson et al. (1987) Cell 51:803-812). Such subclones had a flat cell shape and a reduced ability for anchorage-independent growth. Likewise, transfection with NRAS siRNAs renders HT1080 a flatter shape (FIG. 13). As demonstrated in FIG. 5B and by Paterson et al. (Cell (1987) 51:803-812), however, such NRAS-depleted HT1080 was still viable and kept proliferation in vitro, suggesting the presence of other oncogene(s) in addition to NRAS(Q61K). Therefore, NRAS(Q61K) and RAC1(N92I) likely cooperate to fully transform this fibrosarcoma.

Regarding the coexistence of mutations within RAC family proteins and RAS-RAF-MAPK proteins, two studies independently reported recurrent P29S mutation of RAC1 in melanoma (Krauthammer et al. (2012) Nat. Genet. 44:1006-1014; Hodis et al. (2012) Cell 150:251-263). Of note, BRAF (V600E) was also detected in four of six and in two of seven of the RAC mutation-positive melanomas, respectively. These observations, together with the findings with HT1080 cells, thus indicate that activating mutations of RAC1 and those of the RAS-RAF-signaling pathway are not mutually exclusive. Members of the RAC subfamily of GTPases show a high level of sequence identity in humans. The amino acid sequence of RAC1 is thus 92% identical to that of RAC2 or RAC3. Furthermore, all of the amino acid residues of RAC1 or RAC2 found to be mutated in cancer (Table 3) are completely conserved among RAC1, RAC2, and RAC3. Thus, transforming RAC3 mutants with similar nonsynonymous mutations might also exist in human cancer, although such mutations were not detected in the current screening. None of the frequent mutation sites in RAS family proteins (G12, G13, and Q61) were found to be affected in RAC1 or RAC2, although an artificial G12V mutant of RAC1 did manifest constitutive GTP loading and transforming potential. Given that RAC proteins perform intracellular functions (such as orchestration of the actin cytoskeleton) that are distinct from those of RAS family members, RAC-driven activation of specific intracellular pathways may be advantageous for cancer development in vivo.

Given that activation mutations of RAC1 or RAC2 in cell lines from sarcoma (HT1080), triple-negative breast cancer (MDA-MB-157 and HCC1143), and the blast crisis stage of CML (KCL-22) were identified, deep sequencing of RAC1, RAC2, and RAC3 cDNAs was performed with GAIIx for specimens of triple-negative breast cancer (n=66), of RAC1 and RAC2 cDNAs for specimens of CML in blast crisis (n=43), and of BCR-ABL1-positive acute lymphoblastic leukemia (n=31), as well as of RAC1 cDNAs for specimens of sarcoma (n=53). Nonsynonymous mutations were not detected among these RAC cDNAs.

The results described herein demonstrate that RAC proteins have the potential to become oncogenic through amino acid substitution in a wide array of cancers. Although such RAC mutations may occur at a low frequency, the recent studies of Krauthammer et al. (Nat. Genet. (2012) 44:1006-1014) and Hodis et al. (Cell (2012) 150:251-26324) suggest that they may be enriched in melanoma (about 5%). Given that HT1080 cells are highly addicted to the increased activity of RAC1(N92I), the targeting of oncogenic RAC proteins or their downstream effectors with small compounds or RNAi may prove to be an effective approach to the treatment of cancer harboring such oncoproteins.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated nucleic acid molecule encoding a mutant RAC polypeptide, or a fragment thereof, wherein the mutant RAC polypeptide comprises one or more substitutions of an amino acid in the wild-type RAC polypeptide that renders the mutant RAC polypeptide constitutively active and oncogenic.

2. The isolated nucleic acid molecule of claim 1, wherein mutant RAC polypeptide activity is selected from the group consisting of hydrolyzing guanosine triphosphate (GTP), regulating cell growth, regulating the cell cycle, regulating epithelial differentiation, reorganizing the cellular cytoskeleton, and activating protein kinases.

3. The isolated nucleic acid molecule of any preceding claim, wherein the RAC polypeptide is selected from the group consisting of RAC1, RAC1b, RAC2, and RAC3, or a fragment thereof.

4. The isolated nucleic acid molecule of any preceding claim, wherein the wild-type RAC polypeptide is selected from the group consisting of polypeptides having at least 80% identity over the entire length with the amino acid sequences of SEQ ID NOs: 2, 4, 6, and 8.

5. The isolated nucleic acid molecule of any preceding claim, wherein the wild-type RAC polypeptide is selected from the group consisting of polypeptides having the amino acid sequences of SEQ ID NOs: 2, 4, 6, and 8.

6. The isolated nucleic acid molecule of any preceding claim, wherein the one or more amino acid substitutions is selected from the group consisting of RAC1(N92I), RAC1(C157Y), RAC1(P179L), RAC2(I121M), RAC2(P29Q), RAC2(D47Y), and RAC2(P106H).

7. The isolated nucleic acid molecule of any preceding claim, wherein the nucleic acid molecule has at least 80% identity to the nucleic acid sequence of SEQ ID NOs 1, 3, 5, or 7 and encodes one or more amino acid substitutions selected from the group consisting of RAC1(N92I), RAC1(C157Y), RAC1(P179L), RAC2(I121M), RAC2(P29Q), RAC2(D47Y), and RAC2(P106H).

8. An isolated nucleic acid molecule which hybridizes to the nucleic acid molecule of any preceding claim under stringent hybridization conditions.

9. An isolated nucleic acid molecule comprising a nucleotide sequence which is complementary to the nucleic acid of any preceding claim.

10. The isolated nucleic acid molecule of any preceding claim, wherein the nucleic acid molecule further encodes a heterologous polypeptide.

11. A vector comprising the nucleic acid molecule of any preceding claim.

12. The vector of claim 11, wherein the vector is an expression vector.

13. A host cell transfected with the expression vector of claim 11 or 12.

14. A method of producing a polypeptide comprising culturing the host cell of claim 13 in a cell culture medium to thereby produce the polypeptide.

15. An isolated mutant RAC polypeptide, or a fragment thereof, selected from the group consisting of polypeptides encoded by an isolated nucleic acid molecule of any claims 1 to 10.

16. An antibody which selectively binds to a polypeptide of claim 15.

17. A method of detecting the presence of a polypeptide of claim 15 in a sample comprising:
a) contacting the sample with a compound which selectively binds to the polypeptide; and
b) determining whether the compound binds to the polypeptide in the sample to thereby detect the presence of the polypeptide in the sample.

18. A method of determining whether a subject has cancer, comprising obtaining a biological sample from the subject, and comparing:
a) the amount, structure, subcellular localization, and/or activity of at least one mutant RAC marker of any preceding claim in a subject sample; and
b) the amount, structure, subcellular localization, and/or activity of the at least one mutant RAC marker in a control,
wherein a significant increase in the amount, structure, subcellular localization, and/or activity of the at least one marker in the sample and the amount, structure, subcellular localization, and/or activity in the control indicates that the subject has cancer.

19. A method of prognosing a subject having cancer, comprising obtaining a biological sample from the subject, and comparing:
a) the amount, structure, subcellular localization, and/or activity of at least one mutant RAC marker of any preceding claim in a subject sample; and
b) the amount, structure, subcellular localization, and/or activity of the at least one mutant RAC marker in a control,
wherein a significant increase in the amount, structure, subcellular localization, and/or activity of the at least one marker in the sample and the amount, structure, subcellular localization, and/or activity in the control is an indication that the subject has an unfavorable prognosis.

20. The method of claim 18 or 19, further comprising the step of providing the subject with a therapeutic treatment suitable to treat the cancer.

21. The method of any of claims 18 to 20, wherein the control is a sample from the subject obtained from the subject at an earlier point in time relative to the subject sample.

22. The method of any of claims 18 to 20, wherein the control is determined from a non-cancerous cell sample from the subject or member of the same species to which the subject belongs.

23. The method of any of claims 18 to 20, wherein the control amount, subcellular localization, structure, and/or activity is the wild type amount, subcellular localization, structure, and/or activity in the species to which the subject belongs.

24. The method of any of claims 18 to 20, wherein the control and/or subject sample is obtained before the subject has received adjuvant chemotherapy.

25. The method of any of claims 18 to 20, wherein the control and/or subject sample is obtained after the subject has received adjuvant chemotherapy.

26. The method of any of claims 17 to 25, wherein the sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow.

27. The method of any of claims 18 to 25, wherein the cancer is selected from the group consisting of breast cancer, ovarian cancer, transitional cell bladder cancer, bronchogenic lung cancer, thyroid cancer, pancreatic cancer, prostate cancer, uterine cancer, testicular cancer, gastric cancer, soft tissue and osteogenic sarcomas, neuroblastoma, Wilms' tumor, malignant lymphoma (Hodgkin's and non-Hodgkin's), acute myeloblastic leukemia, acute lymphoblastic leukemia, Kaposi's sarcoma, Ewing's tumor, refractory multiple myeloma, and squamous cell carcinomas of the head, neck, cervix, and vagina.

28. The method of any preceding claim, wherein the amount of the marker is determined by determining the level of expression or copy number of the marker.

29. The method of claim 28, wherein the copy number is determined by using at least one technique selected from the group consisting of fluorescence in situ hybridization (FISH), quantitative PCR (qPCR), comparative genomic hybridization (CGH), and single-nucleotide polymorphism (SNP) array.

30. The method of claim 28, wherein the level of expression of the marker in the sample is assessed by detecting the presence in the sample of a protein corresponding to the marker.

31. The method of claim 30, wherein the protein is detected using a reagent selected from the group consisting of an antibody, an antibody derivative, and an antibody fragment.

32. The method of claim 28, wherein the level of expression of the marker in the sample is assessed by detecting the presence in the sample of a transcribed polynucleotide or portion thereof, wherein the transcribed polynucleotide comprises the marker.

33. The method of claim 32, wherein the transcribed polynucleotide is an mRNA or a cDNA.

34. The method of claim 28, wherein determining the level of expression of the marker comprises the use of at least one technique selected from the group consisting of Northern blot analysis, reverse transcriptase PCR, real-time PCR, RNAse protection, and microarray analysis.

35. The method of claim 28, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

36. The method of claim 8 or 35, wherein stringent hybridization conditions are incubation at 45 deg C in 6× sodium chloride/sodium citrate (SSC), followed by washing in 0.2×SSC and 0.1% SDS at 50-60 deg C.

37. The method of claim 20, wherein the outcome of treatment is measured by at least one criteria selected from the group consisting of survival until mortality, pathological complete response, clinical complete remission, clinical partial remission, clinical stable disease, recurrence-free survival, metastasis free survival, and disease free survival.

38. A kit for determining whether a subject has cancer or for prognosing the outcome of treatment of a subject with cancer, comprising a reagent for assessing the copy number of one or more mutant RAC markers of any preceding claim.

39. A kit for determining whether a subject has cancer or for prognosing the outcome of treatment of a subject with cancer, comprising a reagent for assessing the amount, structure, subcellular localization, and/or activity of one or more mutant RAC markers of any preceding claim.

40. The kit of claim 38 or 39, wherein the reagent is selected from the group consisting of a nucleic acid molecule that hybridizes with the at least one mutant RAC marker.

41. The kit of claim 38 or 39, wherein the reagent is selected from the group consisting of an antibody, and antibody derivative, and an antibody fragment.

42. A kit for treating a subject afflicted with cancer comprising an agent which changes the subcellular localization of or modulates the amount and/or activity of a gene or protein corresponding to one or more mutant RAC markers of any preceding claim.

43. The kit of claim 42, further comprising one or more additional anti-cancer agents.
